(19) United States
(12) Patent Application Publication
(10) Pub. No.: US 2024/0254117 A1
(43) Pub. Date: Aug. 1, 2024

(54) PYRIMIDO-HETEROCYCLIC COMPOUNDS, AND PREPARATION METHOD THEREFOR AND USE THEREOF

(71) Applicant: SHANGHAI RINGENE BIOPHARMA CO., LTD., Shanghai (CN)

(72) Inventors: Huixin WAN, Shanghai (CN); Yazhou WANG, Shanghai (CN); Jingui MA, Shanghai (CN); Yahui WANG, Shanghai (CN); Chuantao ZHA, Shanghai (CN)

(21) Appl. No.: 18/259,306

(22) PCT Filed: Dec. 24, 2021
(86) PCT No.: PCT/CN2021/141359

§ 371 Date: Jun. 26, 2023

(30) Foreign Application Priority Data
Dec. 27, 2020 (CN) 202011571285.6
Jan. 10, 2021 (CN) 202110027514.6
Feb. 10, 2021 (CN) 202110185495.X
May 8, 2021 (CN) 202110500623.5
Sep. 9, 2021 (CN) 202111058245.6

(51) Int. Cl. C07D 471/04; A61K 31/519; A61K 31/5377; A61K 31/5386; A61P 35/00; C07D 471/14; C07D 519/00

(52) U.S. Cl. CPC C07D 471/04; A61K 31/519; A61K 31/5377; A61K 31/5386; A61P 35/00; C07D 471/14; C07D 519/00

(57) ABSTRACT

Provided are a class of pyrimido-heterocyclic compounds, and a preparation method therefor and the use thereof. Specifically provided are a pyrimido-heterocyclic compound represented by general formula I-1 or I-2, or a pharmaceutically acceptable salt, an enantiomer, a diastereomer, a tautomer, a torsional isomer, a solvate, a polymorph, or a prodrug thereof, a preparation method therefor, and the pharmaceutical use thereof. Each group is as defined in the description.

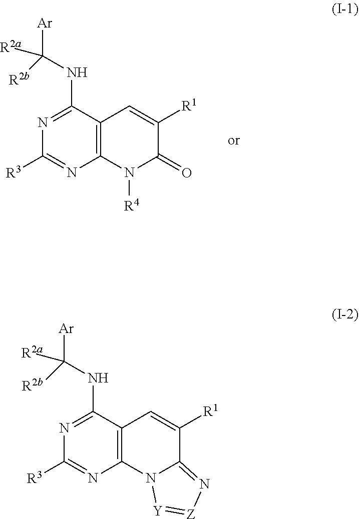

### TECHNICAL FIELD

The present invention belongs to the field of medicinal chemistry, and specifically relates to a class of pyrimido heterocyclic compounds, which have better SOS1 inhibitory activity and can be used for preparing therapeutic and preventive drugs for treating diseases associated with activity or expression or mutation of Ras.

### BACKGROUND ART

Ras protein is a key regulator of normal cell growth and malignant transformation, including cell proliferation, survival and invasion, tumor angiogenesis and metastasis (Downward, Nature Rev. Cancer, 3, 11-22(2003)). In most human tumors, the Ras protein is aberrantly activated due to mutations in the ras gene itself or in upstream or downstream Ras pathway components, or other alterations in Ras signaling. Such mutations reduce the ability of RAS family GTPases hydrolyzing GTP, keeping the molecular switch in an active GTP-bound form that drives unexamined oncogenic downstream signaling. One strategy to reduce active RAS levels is to target guanine nucleotide exchange factors (GEFs), which allow RAS to cycle from an inactive GDP-bound state to an active GTP-bound form. By preventing the formation of the KRAS-SOS1 complex, SOS1 inhibitors block the reloading of KRAS with GTP, resulting in antiproliferative activity. Inhibition of SOS1 may represent a viable approach to target RAS-driven tumors.

Ras-driven cancer remains one of the most clinically intractable diseases, and new treatment and prevention strategies are urgently needed for this cancer (Stephen et al., Cancer Cell, 25,272-281(2014)). The discovery of Ras selective targeting drugs has been ongoing for many years in global academia and industry, but none has been approved for marketing to date (Spiegel, et al., Nature Chem. Biol., 10,613-622(2014)). In the last two years, targeted drugs aiming at Ras-driven have entered the clinical trial stage one after another, and have shown good initial efficacy, and the results are encouraging.

Therefore, there is an urgent need for more therapeutic drugs with unique mechanism, high efficiency and low toxicity to enter the clinic for Ras-driven tumors, and the discovery and search for Ras-targeted drugs with high efficiency, low toxicity and novel structure is still a hot field in the industry.

### SUMMARY OF THE INVENTION

One of the technical problems to be solved by the present invention is to provide a novel SOS1 inhibitor for preparing a tumor therapeutic drug.

The solutions to the above technical problem are as follows:

In one aspect, it provides a pyrimido heterocyclic compound as shown in formula (I-1) or (1-2), or a pharmaceutically acceptable salt thereof, or an enantiomer, a diastereomer, a tautomer, a torsional isomer, a solvate, a polymorph or a prodrug thereof,

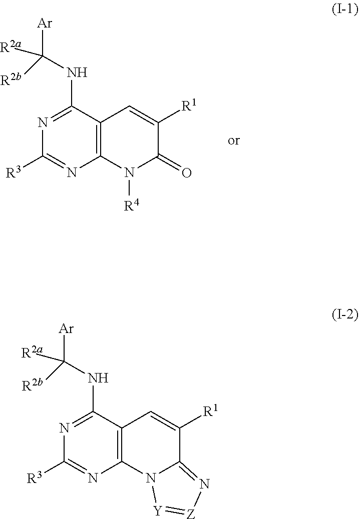

wherein.
R¹ is independently selected from C₁-C₁₀ alkyl, C₁-C₁₀ haloalkyl, C₂-C₁₀ alkenyl, C₂-C₁₀ alkynyl, C₃-C₁₂ cycloalkyl, C₄-C₁₂ cycloalkenyl, 3-12 membered heterocycloalkyl, 5-12 membered aryl or 5-12 membered heteroaryl, —OC₁-C₁₀ alkyl, —NHC₁-C₁₀ alkyl, —N (C₁-C₁₀ alkyl)(C₁-C₁₀ alkyl), —NH (C₃-C₁₂ cycloalkyl), —NH (3-12 membered heterocycloalkyl), —O(C₃-C₁₂ cycloalkyl), —O (3-12 membered heterocycloalkyl), —SC₁-C₁₀ alkyl, —SOC₁-C₁₀ alkyl, —SO₂C₁-C₁₀ alkyl, carbocycle or heteroatom-containing spiro ring/bridged ring/fused ring, wherein the C₁-C₁₀ alkyl, C₂-C₁₀ alkenyl, C₂-C₁₀ alkynyl, C₃-C₁₂ cycloalkyl, C₄-C₁₂ cycloalkenyl, 3-12 membered heterocycloalkyl, 5-12 membered aryl or 5-12 membered heteroaryl, carbocycle or heteroatom-containing spiro ring/bridged ring/fused ring may be optionally substituted with 1 to 3 Rn; or two of the above Rn may form 3-12 membered saturated or partially unsaturated, or aromatic ring system through carbon chain or heteroatom; Rn is independently selected from hydrogen, deuterium, halogen, cyano, nitro, amido, sulfonamido, hydroxyl, amino, ureido, phosphoryl, alkylphosphoxy, alkylsilyl, C₁-C₆ alkyl, C₁-C₆ alkoxy, haloalkyl, haloalkoxy, C₁-C₆ monoalkylamino, C₁-C₆ dialkylamino, alkenyl, alkynyl, 3-8 membered cycloalkyl or heterocycloalkyl, C₁-C₆ alkyl-S—, C₁-C₆ alkyl-SO—, C₁-C₆ alkyl-SO₂—, and the like;
R^{2a} and R^{2b} are each independently selected from hydrogen, deuterium, halogen, C₁-C₆ alkyl, 3-8 membered cycloalkyl or heterocycloalkyl; and R^{2a} and R^{2b}, or R^{2a} and substituent R^{m} on Ar may form a 3-8 membered saturated or partially unsaturated or unsaturated ring system through a carbon chain or heteroatom;
R³ and R⁴ are each independently H, deuterium, halogen, hydroxyl, amino, cyano, C₁-C₆ alkyl, C₁-C₆ haloalkyl, C₁-C₆ alkoxy, C₁-C₆ haloalkoxy, C₁-C₆ alkylamino, 3-8 membered cycloalkyl or heterocycloalkyl, C₂-C₄ alkenyl, C₂-C₄ alkynyl, 5-10 membered aromatic ring or aromatic heterocyclyl;
Y and Z are each independently selected from N or CR⁵, R⁵ is selected from hydrogen, deuterium, halogen, cyano, C₁-C₆ alkyl, 3-8 membered cycloalkyl or heterocycloalkyl;
Ar is selected from 5-12 membered monocyclic or bicyclic aryl or heteroaryl, which may be substituted by one or more R^{m}, R^{m} is selected from the group consisting of
hydrogen, deuterium, halogen, cyano, nitro, substituted or unsubstituted amido, substituted or unsubstituted sulfonamido, hydroxyl, amino, ureido, phosphoryl, alkylphosphoxy, alkylsilyl, C₁-C₁₀ alkyl, C₁-C₁₀ alkoxy, C₁-C₁₀ alkoxyalkyl, C₁-C₁₀ haloalkyl, C₁-C₁₀ haloalkoxy, C₁-C₁₀ haloalkoxyalkyl, C₁-C₁₀ monoalkylamino, C₁-C₁₀ dialkylamino, C₁-C₁₀ monoalkylaminoalkyl, C₁-C₁₀ dialkylaminoalkyl, C₁-C₁₀ alkenyl, C₁-C₁₀ alkynyl, 3-12 membered cycloalkyl or heterocycloalkyl, 3-12 membered cycloalkyl or heterocycloalkyl alkyl, C₁-C₁₀ alkyl-S—, C₁-C₁₀ alkyl-SO—, C₁-C₁₀ alkyl-SO₂—, substituted or unsubstituted 5-12 membered aryl or heteroaryl, and the like, or two of the above R^{m} may form a 3-12 membered saturated or partially unsaturated, or aromatic ring system through a carbon chain or heteroatom;
one or more hydrogen atoms on any of the above groups may be substituted by a substituent selected from the group consisting of (including but not limited to) deuterium, halogen, hydroxyl, C₁-C₃ alkyl, C₁-C₃ haloalkyl, 3-6 membered cycloalkyl or heterocycloalkyl, oxo (═O), C₁-C₃ alkoxy, C₁-C₁₀ monoalkylaminoalkyl, C₁-C₁₀ dialkylaminoalkyl, 3-6 membered cycloalkyl C₁-C₃ alkyl-, 3-6 membered heterocycloalkyl C₁-C₃ alkyl-, amino 3-6 membered cycloalkyl-, amino 3-6 membered heterocycloalkyl-, C(═O)(3-6 membered heterocyclyl) C₁-C₃ alkyl (e.g.

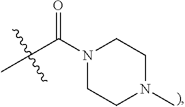 [SMILES: *C(=O)N1CCN(C)CC1]

C(═O)C₁-C₃ alkyl, C(═O)C₁-C₁₀ monoalkylaminoalkyl, C(═O) C₁-C₁₀ dialkylamioalkyl, C(═O)C₁-C₃ alkyl, C(═O) amino C₁-C₁₀ monoalkylOH, C(═O) amino C₁-C₁₀ dialkylOH (e.g.

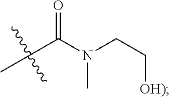

wherein, the heteroaryl contains 1-3 heteroatoms selected from the group consisting of N, O, P or S, the heterocycloalkyl contains 1-3 heteroatoms selected from the group consisting of N, O, P or S, and the ring system comprises a saturated or partially unsaturated ring system such as spiro ring, bridged ring, fused ring, and fused ring.

A pyrimido heterocyclic compound having formula (I-1) or (I-2), or a pharmaceutically acceptable salt thereof, or an enantiomer, a diastereomer, a tautomer, a torsional isomer, a solvate, a polymorph or a prodrug thereof,

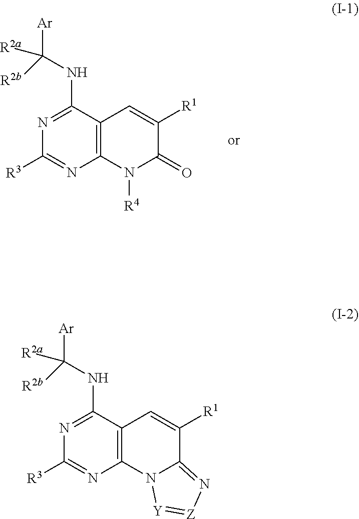

wherein:
R¹ is independently selected from C₁-C₁₀ alkyl, C₁-C₁₀ haloalkyl, C₂-C₁₀ alkenyl, C₂-C₁₀ alkynyl, C₃-C₁₂ cycloalkyl, C₄-C₁₂ cycloalkenyl, 3-12 membered heterocycloalkyl, 5-12 membered aryl or 5-12 membered heteroaryl, —OC₁-C₁₀ alkyl, —NHC₁-C₁₀ alkyl, —N (C₁-C₁₀ alkyl)(C₁-C₁₀ alkyl), —SC₁-C₁₀ alkyl, —SOC₁-C₁₀ alkyl, —SO₂C₁-C₁₀ alkyl, carbocycle or heteroatom-containing spiro ring/bridged ring/fused ring, wherein the C₁-C₁₀ alkyl, C₂-C₁₀ alkenyl, C₂-C₁₀ alkynyl, C₃-C₁₂ cycloalkyl, C₄-C₁₂ cycloalkenyl, 3-12 membered heterocycloalkyl, 5-12 membered aryl or 5-12 membered heteroaryl, carbocycle or heteroatom-containing spiro ring/bridged ring/fused ring may be optionally substituted with 1-3 Rn; or two of the above Rn may form 3-12 membered saturated or partially unsaturated, or aromatic ring system through carbon chain or heteroatom; Rn is selected from hydrogen, deuterium, halogen, cyano, nitro, amido, sulfonamido, hydroxyl, amino, ureido, phosphoryl, alkylphosphoxy, alkylsilyl, C₁-C₆ alkyl, C₁-C₆ alkoxy, haloalkyl, haloalkoxy, C₁-C₆ monoalkylamino, C₁-C₆ dialkylamino, alkenyl, alkynyl, 3-8 membered cycloalkyl or heterocycloalkyl, C₁-C₆ alkyl-S—, C₁-C₆ alkyl-SO—, C₁-C₆ alkyl-SO₂—, and the like;
R^{2a} and R^{2b} are each independently selected from hydrogen, deuterium, halogen, C₁-C₆ alkyl, 3-8 membered cycloalkyl or heterocycloalkyl; and R^{2a} and R^{2b} or R^{2a} and substituent R^{m} on Ar may form a 3-8 membered saturated or partially unsaturated or unsaturated ring system through a carbon chain or heteroatom;
R³ and R⁴ are each independently H, deuterium, halogen, hydroxyl, amino, cyano, C₁-C₆ alkyl, C₁-C₆ haloalkyl, C₁-C₆ alkoxy, C₁-C₆ haloalkoxy, C₁-C₆ alkylamino, 3-8 membered cycloalkyl or heterocycloalkyl, C₂-C₄ alkenyl, C₂-C₄ alkynyl, 5-10 membered aromatic ring or aromatic heterocyclyl;
Y and Z are each independently selected from N or CR⁵, R⁵ is selected from hydrogen, deuterium, halogen, cyano, C₁-C₆ alkyl, 3-8 membered cycloalkyl or heterocycloalkyl;
Ar is selected from 5-12 membered monocyclic or bicyclic aryl or heteroaryl, which may be substituted by one or more groups selected from the group consisting of
hydrogen, deuterium, halogen, cyano, nitro, substituted or unsubstituted amido, substituted or unsubstituted sulfonamido, hydroxyl, amino, ureido, phosphoryl, alkylphosphoxy, alkylsilyl, C₁-C₁₀ alkyl, C₁-C₁₀ alkoxy, C₁-C₁₀ alkoxyalkyl, C₁-C₁₀ haloalkyl, C₁-C₁₀ haloalkoxy, C₁-C₁₀ haloalkoxyalkyl, C₁-C₁₀ monoalkylamino, C₁-C₁₀ dialkylamino, C₁-C₁₀ monoalkylaminoalkyl, C₁-C₁₀ dialkylaminoalkyl, C₁-C₁₀ alkenyl, C₁-C₁₀ alkynyl, 3-12 membered cycloalkyl or heterocycloalkyl, 3-12 membered cycloalkyl or heterocycloalkyl alkyl, C₁-C₁₀ alkyl-S—, C₁-C₁₀ alkyl-SO—, C₁-C₁₀ alkyl-SO₂—, substituted or unsubstituted 5-12 membered aryl or heteroaryl, and the like, or two of the above R^{m} may form a 3-12 membered saturated or partially unsaturated, or aromatic ring system through a carbon chain or heteroatom;
one or more hydrogen atoms on any of the above groups may be substituted by a substituent selected from the group consisting of (including but not limited to) deuterium, halogen, C₁-C₃ alkyl, C₁-C₃ haloalkyl, 3-6 membered cycloalkyl or heterocycloalkyl; wherein the heteroaryl contains 1-3 heteroatoms selected from the group consisting of N, O, P or S, the heterocycloalkyl contains 1-3 heteroatoms selected from the group consisting of N, O, P or S, and the ring system includes a saturated or partially unsaturated ring system such as a spiro ring, a bridged ring, a fused ring, and a fused ring.

In some preferred embodiments, the pyrimido heterocyclic compound shown in formula (I-1) or (1-2), or pharmaceutically acceptable salt, or its enantiomer, diastereomer, tautomer, torsional isomer, solvate, polymorph or prodrug thereof,

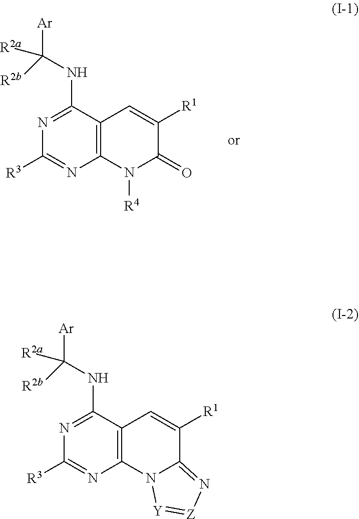

wherein:
R¹ is independently selected from C₁-C₁₀ alkyl, C₁-C₁₀ haloalkyl, C₂-C₁₀ alkenyl, C₂-C₁₀ alkynyl, C₃-C₁₂ cycloalkyl, C₄-C₁₂ cycloalkenyl, 3-12 membered heterocycloalkyl, 5-12 membered aryl or 5-12 membered heteroaryl, —OC₁-C₁₀ alkyl, —NHC₁-C₁₀ alkyl, —N (C₁-C₁₀ alkyl)(C₁-C₁₀ alkyl), —SC₁-C₁₀ alkyl, —SOC₁-C₁₀ alkyl, —SO₂C₁-C₁₀ alkyl, wherein the C₁-C₁₀ alkyl, C₂-C₁₀ alkenyl, C₂-C₁₀ alkynyl, C₃-C₁₂ cycloalkyl, C₄-C₁₂ cycloalkenyl, 3-12 membered heterocycloalkyl, 5-12 membered aryl or 5-12 membered heteroaryl may be optionally substituted with 1-3 Rn; or two of the above Rn may form 3-12 membered saturated or partially unsaturated, or aromatic ring system through carbon chain or heteroatom; Rn is selected from hydrogen, deuterium, halogen, cyano, nitro, amido, sulfonamido, hydroxyl, amino, ureido, phosphoryl, alkylphosphoxy, alkylsilyl, C₁-C₆ alkyl, C₁-C₆ alkoxy, haloalkyl, haloalkoxy, C₁-C₆ monoalkylamino, C₁-C₆ dialkylamino, alkenyl, alkynyl, 3-8 membered cycloalkyl or heterocycloalkyl, C₁-C₆ alkyl-S—, C₁-C₆ alkyl-SO—, C₁-C₆ alkyl-SO₂—, and the like;
R^{2a} and R^{2b} are each independently selected from hydrogen, deuterium, halogen, C₁-C₆ alkyl, 3-8 membered cycloalkyl or heterocycloalkyl; and R^{2a} and R^{2b} may form a 3-6 membered saturated or partially unsaturated or unsaturated ring system through a carbon chain or heteroatom;
R³ and R⁴ are each independently H, deuterium, halogen, hydroxyl, amino, C₁-C₆ alkyl, C₁-C₆ haloalkyl, C₁-C₆ alkoxy, C₁-C₆ alkylamino, 3-8 membered cycloalkyl or heterocycloalkyl, C₂-C₄ alkenyl, C₂-C₄ alkynyl;
Y and Z are each independently selected from N or CR⁵, R⁵ is selected from hydrogen, deuterium, halogen, cyano, C₁-C₆ alkyl, 3-8 membered cycloalkyl or heterocycloalkyl;
Ar is selected from 5-12 membered monocyclic or bicyclic aryl or heteroaryl, which may be substituted by one or more groups selected from the group consisting of hydrogen, deuterium, halogen, cyano, nitro, substituted or unsubstituted amido, substituted or unsubstituted sulfonamido, hydroxyl, amino, ureido, phosphoryl, alkylphosphoxy, alkylsilyl, C₁-C₁₀ alkyl, C₁-C₁₀ alkoxy, C₁-C₁₀ alkoxyalkyl, C₁-C₁₀ haloalkyl, C₁-C₁₀ haloalkoxy, C₁-C₁₀ haloalkoxyalkyl, C₁-C₁₀ monoalkylamino, C₁-C₁₀ dialkylamino, C₁-C₁₀ monoalkylaminoalkyl, C₁-C₁₀ dialkylaminoalkyl, C₁-C₁₀ alkenyl, C₁-C₁₀ alkynyl, 3-12 membered cycloalkyl or heterocycloalkyl, 3-12 membered cycloalkyl or heterocycloalkyl alkyl, C₁-C₁₀ alkyl-S—, C₁-C₁₀ alkyl-SO—, C₁-C₁₀ alkyl-SO₂—, substituted or unsubstituted 5-12 membered aryl or heteroaryl, and the like;
one or more hydrogen atoms on any of the above groups may be substituted by a substituent selected from the group consisting of (including but not limited to) deuterium, halogen, C₁-C₃ alkyl, C₁-C₃ haloalkyl, 3-6 membered cycloalkyl or heterocycloalkyl; wherein the heteroaryl contains 1-3 heteroatoms selected from the group consisting of N, O, P or S, the heterocycloalkyl contains 1-3 heteroatoms selected from the group consisting of N, O, P or S, and the ring system includes a saturated or partially unsaturated ring system such as a spiro ring, a bridged ring, a fused ring, and a fused ring.

In some preferred embodiments, the compound of general formula (I), or a pharmaceutically acceptable salt thereof, or an enantiomer, a diastereomer, a tautomer, a torsional isomer, a solvate, a polymorph or a prodrug thereof is preferably a compound of general formula (II-1) or (II-2),

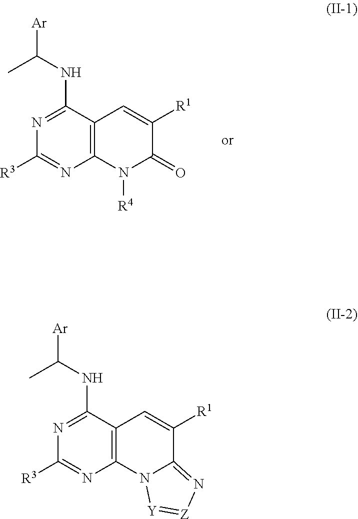

R¹, R³, R⁴, Ar are as defined above.

In some preferred embodiments, R³ is preferably selected from H, Me.

In some preferred embodiments, the compound has the following structure:

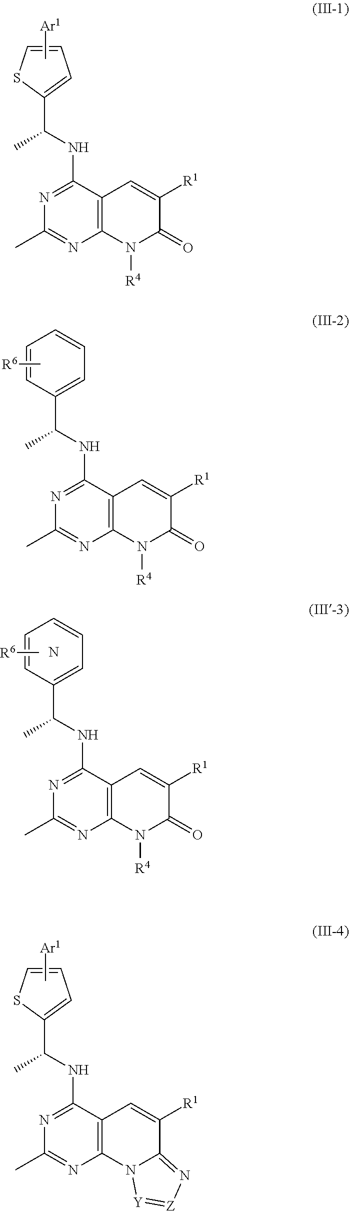

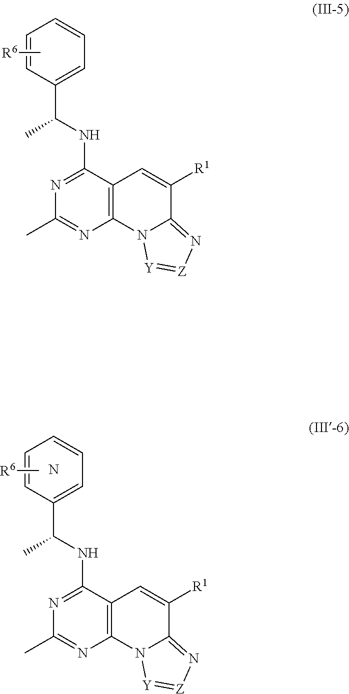

wherein, Ar¹ is preferably selected from a 5-6 membered aromatic or heteroaromatic ring system, and the above ring system may be substituted by 1-5 substituents selected from the group consisting of hydrogen, deuterium, halogen, cyano, nitro, substituted or unsubstituted amido, substituted or unsubstituted sulfonamido, hydroxyl, amino, ureido, phosphoryl, alkylphosphoxy, alkylsilyl, C₁-C₁₀ alkyl, C₁-C₁₀ alkoxy, C₁-C₁₀ alkoxyalkyl, C₁-C₁₀ haloalkyl, C₁-C₁₀ haloalkoxy, C₁-C₁₀ haloalkoxyalkyl, C₁-C₁₀ monoalkylamino, C₁-C₁₀ dialkylamino, C₁-C₁₀ monoalkylaminoalkyl, C₁-C₁₀ dialkylaminoalkyl, C₁-C₁₀ alkenyl, C₁-C₁₀ alkynyl, 3-12 membered cycloalkyl or heterocycloalkyl, 3-12 membered cycloalkyl or heterocycloalkyl alkyl, C₁-C₁₀ alkyl-S—, C₁-C₁₀ alkyl-SO—, C₁-C₁₀ alkyl-SO₂—, substituted or unsubstituted 5-12 membered aryl or heteroaryl, and the like; R⁶ is independently selected from 1-5 substituents selected from the group consisting of hydrogen, deuterium, halogen, cyano, nitro, substituted or unsubstituted amido, substituted or unsubstituted sulfonamido, hydroxyl, amino, ureido, phosphoryl, alkylphosphoxy, alkylsilyl, C₁-C₁₀ alkyl, C₁-C₁₀ alkoxy, C₁-C₁₀ alkoxyalkyl, C₁-C₁₀ haloalkyl, C₁-C₁₀ haloalkoxy, C₁-C₁₀ haloalkoxyalkyl, C₁-C₁₀ monoalkylamino, C₁-C₁₀ dialkylamino, C₁-C₁₀ monoalkylaminoalkyl, C₁-C₁₀ dialkylaminoalkyl, C₁-C₁₀ alkenyl, C₁-C₁₀ alkynyl, 3-12 membered cycloalkyl or heterocycloalkyl, 3-12 membered cycloalkyl or heterocycloalkyl alkyl, C₁-C₁₀ alkyl-S—, C₁-C₁₀ alkyl-SO—, C₁-C₁₀ alkyl-SO₂—, substituted or unsubstituted 5-12 membered aryl or heteroaryl, and the like; R¹, R⁴, Y and Z are as defined above.

In another preferred embodiment, Ar is phenyl or 5-6 membered heteroaryl; more preferably is phenyl, thienyl, pyridyl; wherein the phenyl, 5-6 membered heteroaryl, thienyl, pyridyl can be substituted by one or more R^{m}, R^{m} is selected from the group consisting of
hydrogen, deuterium, halogen, cyano, nitro, substituted or unsubstituted amido, substituted or unsubstituted sulfonamido, hydroxyl, amino, ureido, phosphoryl, alkylphosphoxy, alkylsilyl, C₁-C₁₀ alkyl, C₁-C₁₀ alkoxy, C₁-C₁₀ alkoxyalkyl, C₁-C₁₀ haloalkyl, C₁-C₁₀ haloalkoxy, C₁-C₁₀ haloalkoxyalkyl, C₁-C₁₀ monoalkylamino, C₁-C₁₀ dialkylamino, C₁-C₁₀ monoalkylaminoalkyl, C₁-C₁₀ dialkylaminoalkyl, C₁-C₁₀ alkenyl, C₁-C₁₀ alkynyl, 3-12 membered cycloalkyl or heterocycloalkyl, 3-12 membered cycloalkyl or heterocycloalkyl alkyl, C₁-C₁₀ alkyl-S—, C₁-C₁₀ alkyl-SO—, C₁-C₁₀ alkyl-SO₂—, substituted or unsubstituted 5-12 membered aryl or heteroaryl, and the like, or two of the above R^{m} may form a 3-12 membered saturated or partially unsaturated, or aromatic ring system through a carbon chain or heteroatom;
preferably, R^{m} is selected from: trifluoromethyl, NH₂, methyl,

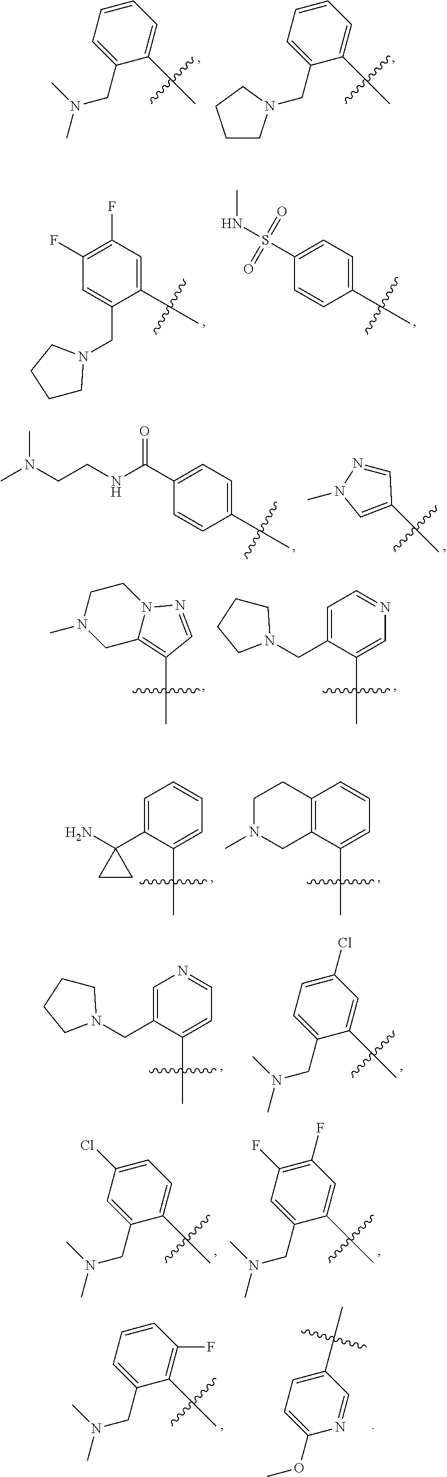

In another some preferred embodiments, the compound having general formula (I), or a pharmaceutically acceptable salt thereof, or an enantiomer, a diastereomer, a tautomer, a torsional isomer, a solvate, a polymorph or a prodrug thereof is preferably a compound having general formula (III-1) to (III-12),

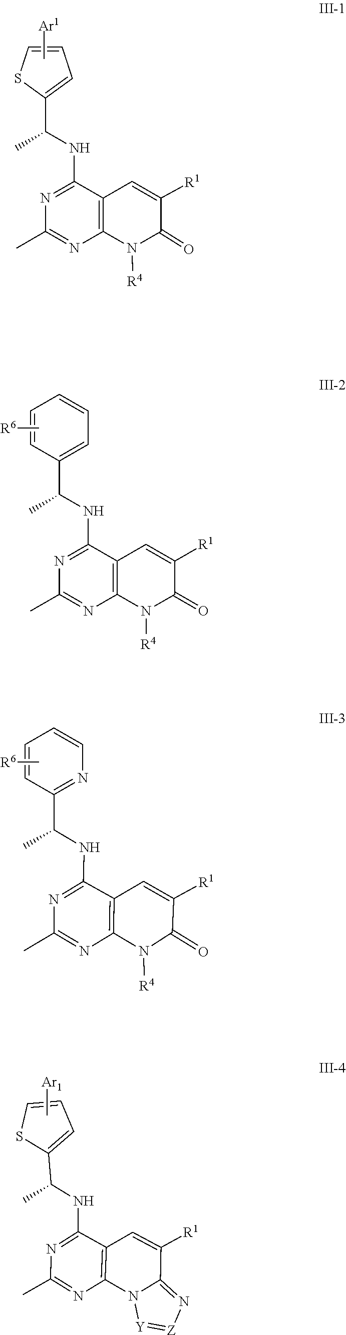

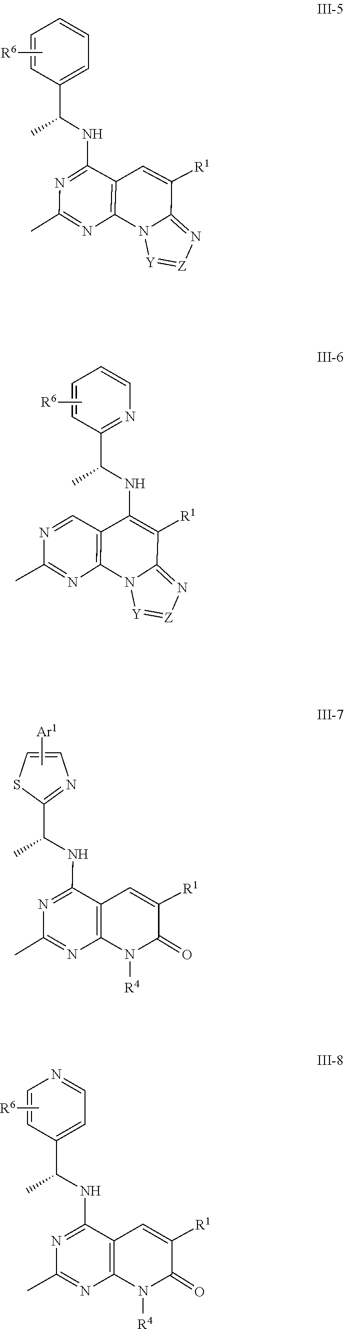

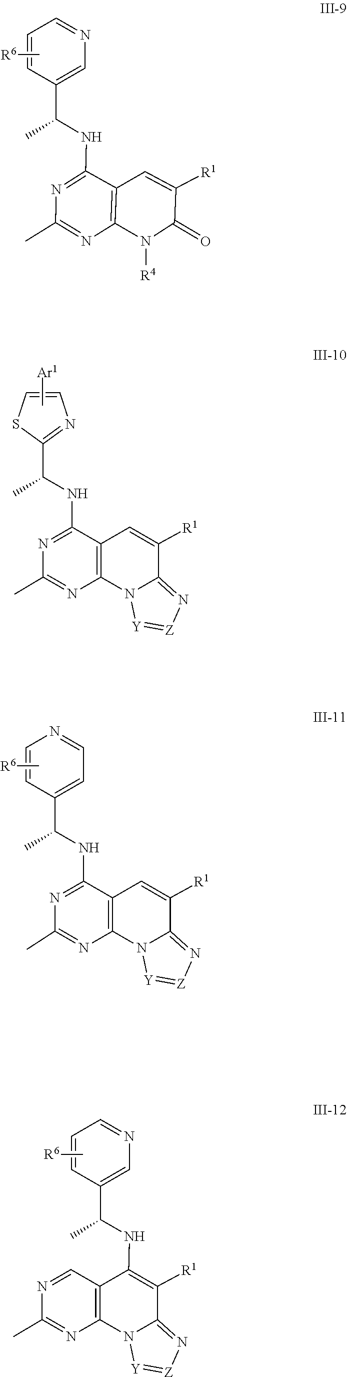

wherein, Ar¹ is preferably selected from a 5-6 membered aromatic or heteroaromatic ring system, and the above ring system may be substituted by 1-5 substituents selected from the group consisting of hydrogen, deuterium, halogen, cyano, nitro, substituted or unsubstituted amido, substituted or unsubstituted sulfonamido, hydroxyl, amino, ureido, phosphoryl, alkylphosphoxy, alkylsilyl, C₁-C₁₀ alkyl, C₁-C₁₀ alkoxy, C₁-C₁₀ alkoxyalkyl, C₁-C₁₀ haloalkyl, C₁-C₁₀ haloalkoxy, C₁-C₁₀ haloalkoxyalkyl, C₁-C₁₀ monoalkylamino, C₁-C₁₀ dialkylamino, C₁-C₁₀ monoalkylaminoalkyl, C₁-C₁₀ dialkylaminoalkyl, C₁-C₁₀ alkenyl, C₁-C₁₀ alkynyl, 3-12 membered cycloalkyl or heterocycloalkyl, 3-12 membered cycloalkyl or heterocycloalkyl alkyl, C₁-C₁₀ alkyl-S—, C₁-C₁₀ alkyl-SO—, C₁-C₁₀ alkyl-SO₂—, substituted or unsubstituted 5-12 membered aryl or heteroaryl, and the like; R⁶ is independently selected from 1-5 substituents selected from the group consisting of hydrogen, deuterium, halogen, cyano, nitro, substituted or unsubstituted amido, substituted or unsubstituted sulfonamido, hydroxyl, amino, ureido, phosphoryl, alkylphosphoxy, alkylsilyl, C₁-C₁₀ alkyl, C₁-C₁₀ alkoxy, C₁-C₁₀ alkoxyalkyl, C₁-C₁₀ haloalkyl, C₁-C₁₀ haloalkoxy, C₁-C₁₀ haloalkoxyalkyl, C₁-C₁₀ monoalkylamino, C₁-C₁₀ dialkylamino, C₁-C₁₀ monoalkylaminoalkyl, C₁-C₁₀ dialkylaminoalkyl, C₁-C₁₀ alkenyl, C₁-C₁₀ alkynyl, 3-12 membered cycloalkyl or heterocycloalkyl, 3-12 membered cycloalkyl or heterocycloalkyl alkyl, C₁-C₁₀ alkyl-S—, C₁-C₁₀ alkyl-SO—, C₁-C₁₀ alkyl-SO₂—, substituted or unsubstituted 5-12 membered aryl or heteroaryl, and the like; R¹, R⁴, Y and Z are as defined above.

In another preferred embodiment, R⁴ is methyl.

In another preferred embodiment, R³ is methyl.

In another preferred embodiment, R^{2a} is H.

In another preferred embodiment, R^{2b} is methyl.

In another preferred embodiment, R^{2a} is H and R^{2b} is methyl.

In another preferred embodiment, both Y and Z are CH.

In another preferred embodiment, the definition of Ar¹ is the same as that of R^{m}.

In another preferred embodiment, the definition of R⁶ is the same as that of R^{m}.

In another preferred embodiment, R^{m} is selected from the group consisting of hydrogen, deuterium, halogen, cyano, nitro, substituted or unsubstituted amido, substituted or unsubstituted sulfonamido, hydroxyl, amino, ureido, phosphoryl, alkylphosphoxy, alkylsilyl, C₁-C₁₀ alkyl, C₁-C₁₀ alkoxy, C₁-C₁₀ alkoxyalkyl, C₁-C₁₀ haloalkyl, C₁-C₁₀ haloalkoxy, C₁-C₁₀ haloalkoxyalkyl, C₁-C₁₀ monoalkylamino, C₁-C₁₀ dialkylamino, C₁-C₁₀ monoalkylaminoalkyl, C₁-C₁₀ dialkylaminoalkyl, C₁-C₁₀ alkenyl, C₁-C₁₀ alkynyl, 3-12 membered cycloalkyl or heterocycloalkyl, 3-12 membered cycloalkyl or heterocycloalkyl alkyl, C₁-C₁₀ alkyl-S—, C₁-C₁₀ alkyl-SO—, C₁-C₁₀ alkyl-SO₂—, substituted or unsubstituted 5-12 membered aryl or heteroaryl, and the like, or two of the above R^{m} may form a 3-12 membered saturated or partially unsaturated, or aromatic ring system through a carbon chain or heteroatom;
one or more hydrogen atoms on any of the above groups may be substituted by a substituent selected from the group consisting of (including but not limited to) hydrogen, deuterium, halogen, cyano, nitro, substituted or unsubstituted amido, substituted or unsubstituted sulfonamido, hydroxyl, amino, ureido, phosphoryl, alkylphosphoxy, alkylsilyl, C₁-C₁₀ alkyl, C₁-C₁₀ alkoxy, C₁-C₁₀ alkoxyalkyl, C₁-C₁₀ haloalkyl, C₁-C₁₀ haloalkoxy, C₁-C₁₀ haloalkoxyalkyl, C₁-C₁₀ monoalkylamino, C₁-C₁₀ dialkylamino, C₁-C₁₀ monoalkylaminoalkyl, C₁-C₁₀ dialkylaminoalkyl, C₁-C₁₀ alkenyl, C₁-C₁₀ alkynyl, 3-12 membered cycloalkyl or heterocycloalkyl, 3-12 membered cycloalkyl or heterocycloalkyl alkyl, C₁-C₁₀ alkyl-S—, C₁-C₁₀ alkyl-SO—, C₁-C₁₀ alkyl-SO₂—, substituted or unsubstituted 5-12 membered aryl or heteroaryl, and the like.

In another preferred embodiment, R¹ is selected from the following group:

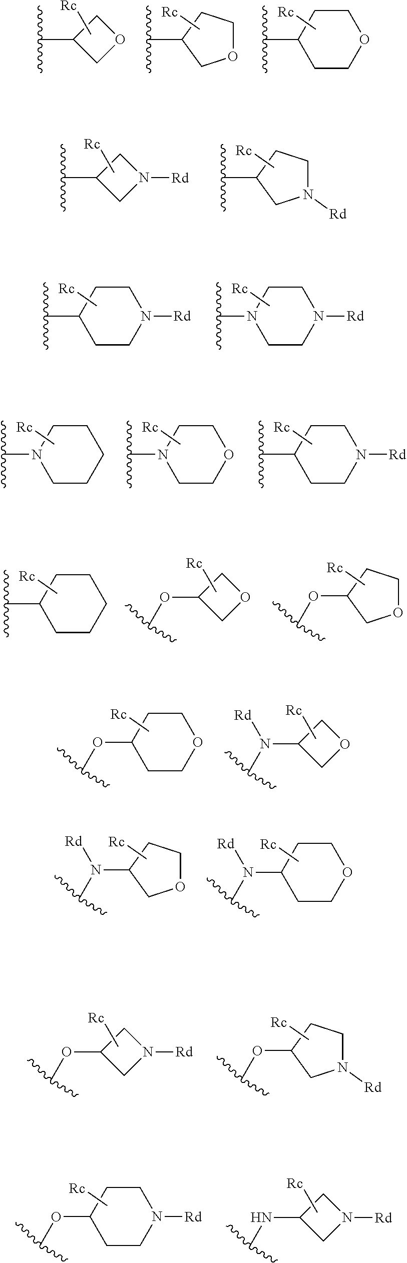

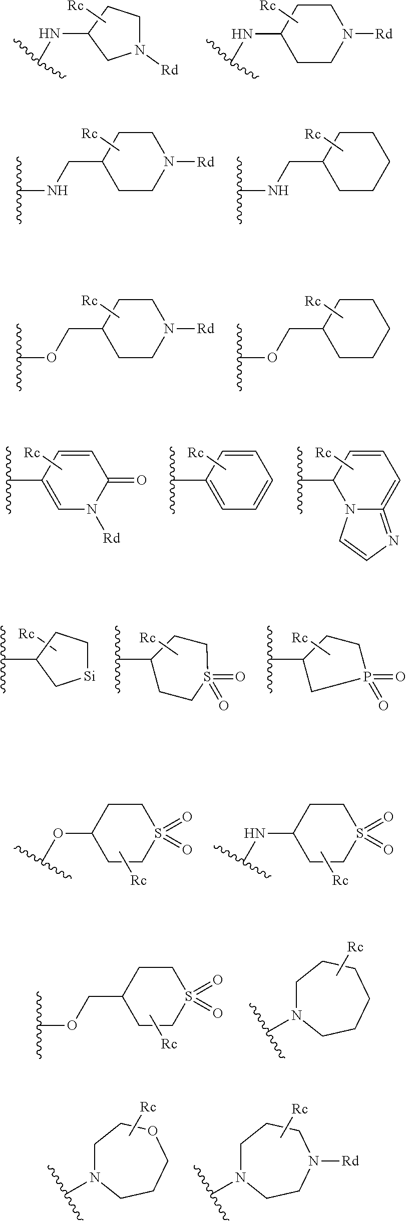

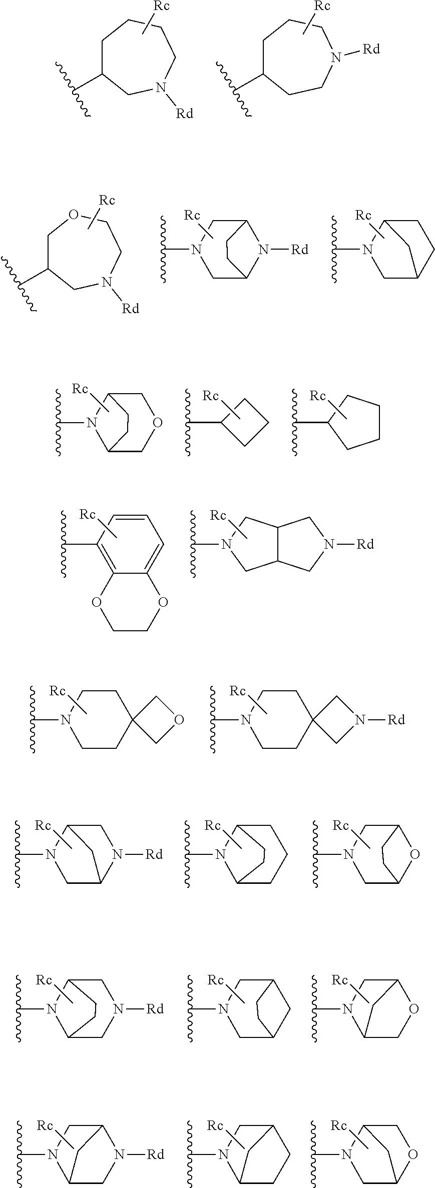

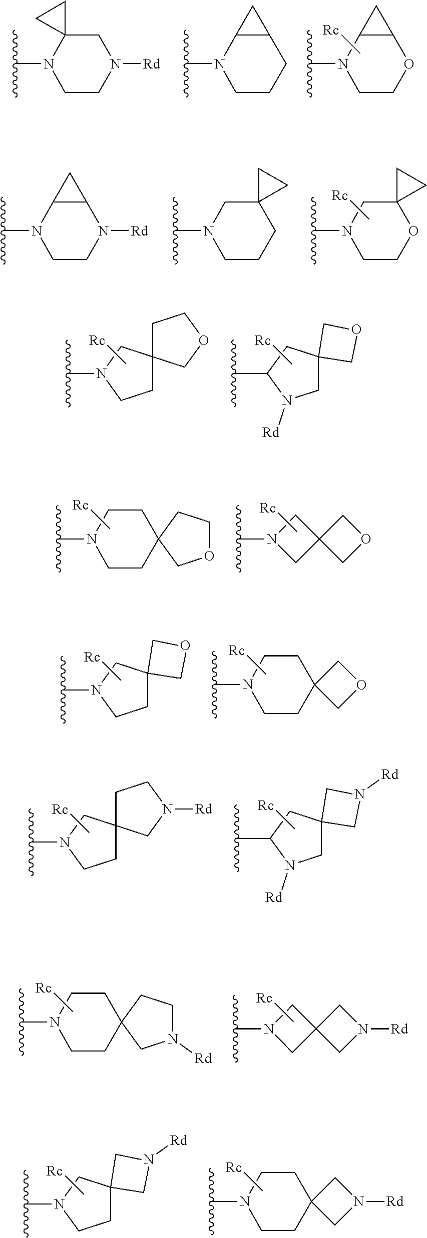

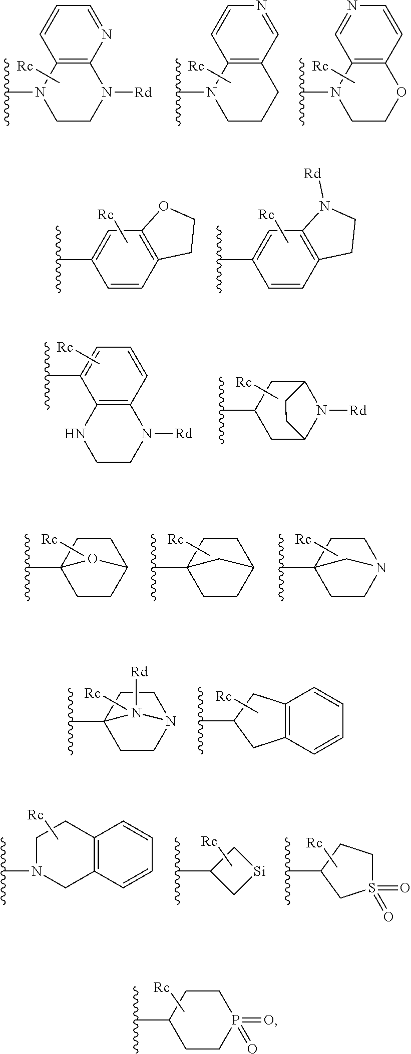

wherein one or more R^{c} are each independently selected from hydrogen, deuterium, halogen, —C₁-C₆ alkyl, —OC₁-C₆ alkyl, cyano, hydroxy, amino, —C₁—C₆ alkyl, —OC₁-C₆ alkyl, —SO₂C₁-C₆ alkyl, —COC₁-C₆ alkyl, —COOC₁-C₆ alkyl, —CONHC₁-C₆ alkyl, —CON (C₁-C₆ alkyl) (C₁-C₆ alkyl), 3-6 membered cycloalkyl or heterocycloalkyl, 5-10 membered aryl or heteroaryl, —C₁-C₆ haloalkyl, —C₁-C₆ haloalkoxy, —C₁-C₆ deuterated alkyl, —C₁-C₆ deuterated alkoxy, —O-3-6 membered cycloalkyl or heterocycloalkyl, —C₁-C₆ alkyl OC₁-C₆ alkyl, —C₁-C₆ alkyl NHC₁-C₆ alkyl, —C₁-C₆ alkyl OH, —C₁-C₆ alkyl N (C₁-C₆ alkyl) (C₁-C₆ alkyl), —C₁-C₆ alkyl 3-6 membered cycloalkyl, —C₁-C₆ alkyl 3-6 membered heterocycloalkyl, C(═O)(3-6 membered heterocyclyl) C₁-C₃ alkyl, or C(═O) amino C₁-C₆ dialkyl OH, and any two Rc can form 3-10 membered saturated or partially unsaturated carbocyclic or heterocyclic ring through carbon chain or heteroatom; R^{d} is independently selected from —C₁-C₆ alkyl, —C₁-C₆ alkyl OC₁-C₆ alkyl, —C₁-C₆ alkyl SC₁-C₆ alkyl, —C₁-C₆ alkyl SOC₁-C₆ alkyl, —C₁-C₆ alkyl SO₂C₁-C₆ alkyl, —COC₁-C₆ alkyl, —COOC₁-C₆ alkyl, —CONHC₁-C₆ alkyl, —CON (C₁-C₆ alkyl)(C₁-C₆ alkyl), 3-6 membered cycloalkyl or heterocycloalkyl, 5-10 membered aryl or heteroaryl, —C₁-C₆ haloalkyl, —C₁-C₆ haloalkoxy, —C₁-C₆ deuterated alkyl, —C₁-C₆ deuterated alkoxy-C₁-C₆ alkyl, —C₁-C₆ alkyl-O-3-6-membered cycloalkyl or heterocycloalkyl, —C₁-C₆ alkyl-NHC₁-C₆ alkyl, —C₁-C₆ alkyl OH, —C₁-C₆ alkyl N (C₁-C₆ alkyl)(C₁-C₆ alkyl), and the like.

In another some preferred embodiments, R¹ is selected from the following groups

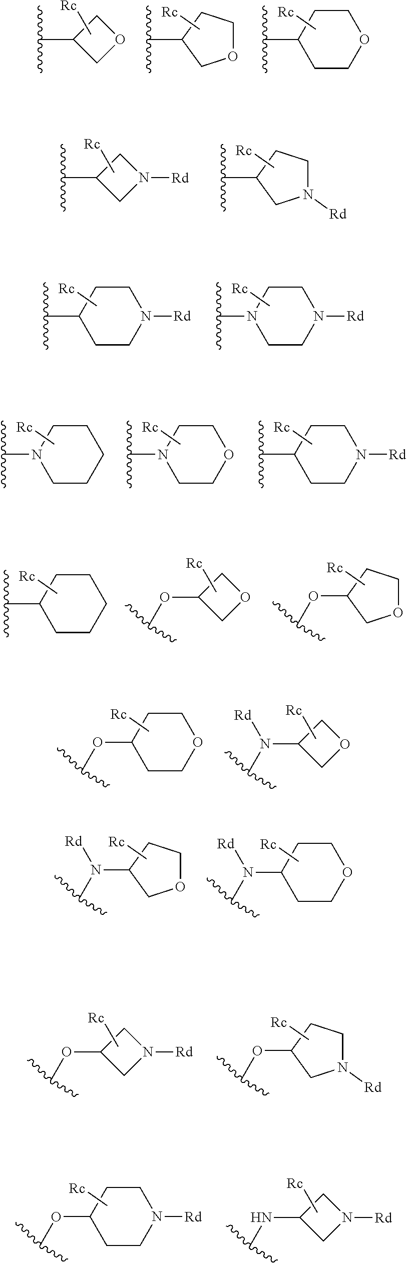

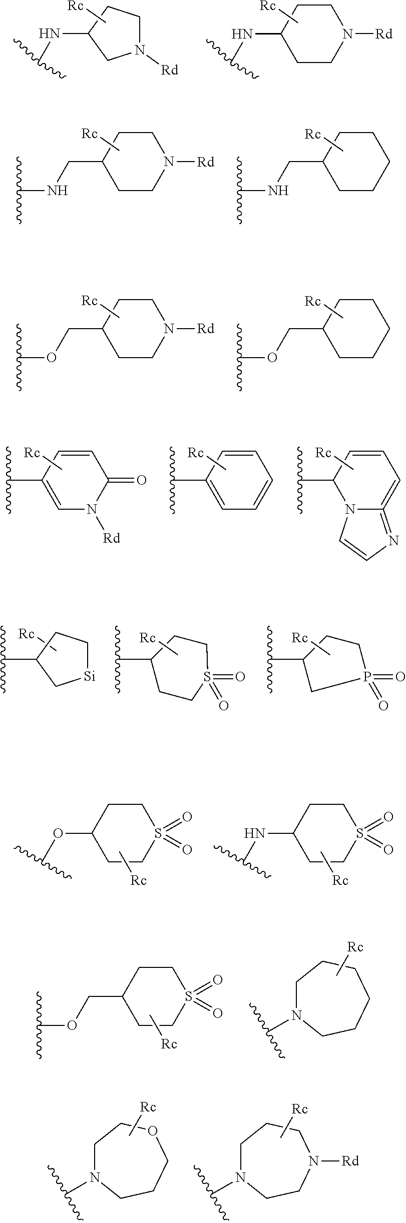

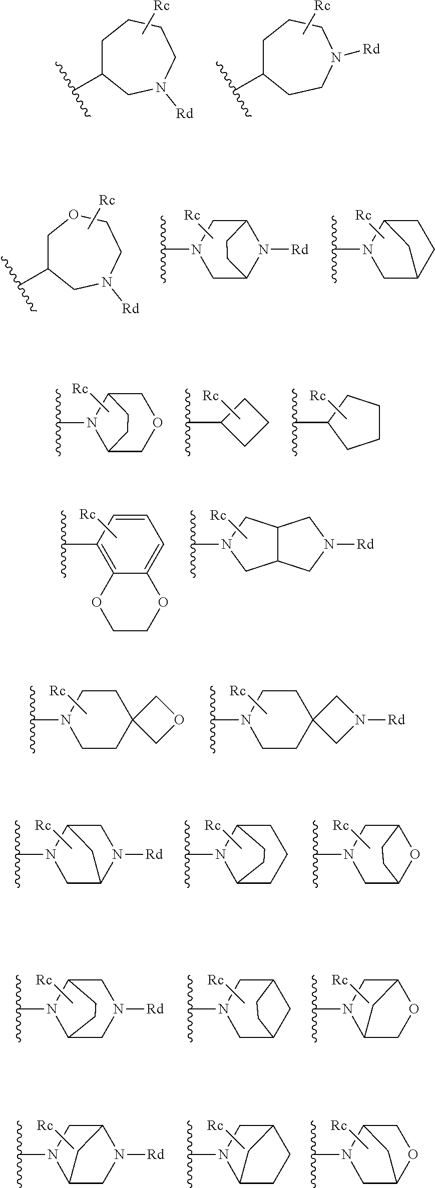

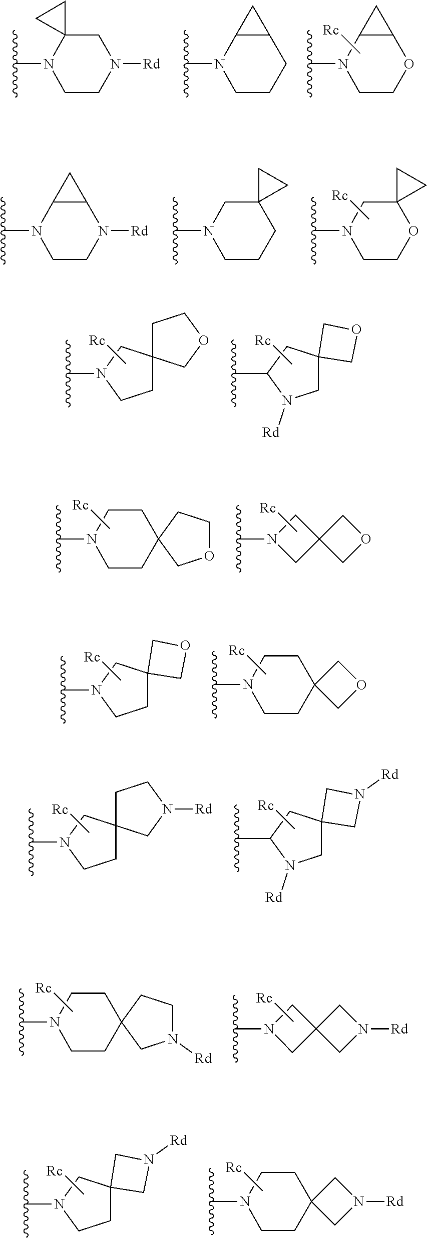

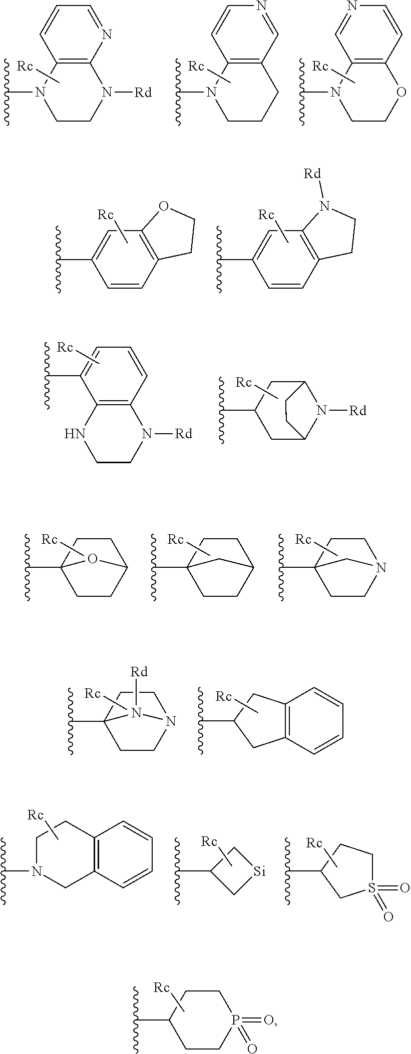

wherein one or more R^{c} are each independently selected from hydrogen, deuterium, halogen, —C₁-C₆ alkyl, —OC₁-C₆ alkyl, cyano, hydroxy, amino, —C₁-C₆ alkyl, —OC₁-C₆ alkyl, —SO₂C₁-C₆ alkyl, —COC₁-C₆ alkyl, —COOC₁-C₆ alkyl, —CONHC₁-C₆ alkyl, —CON (C₁-C₆ alkyl) (C₁-C₆ alkyl), 3-6 membered cycloalkyl or heterocycloalkyl, 5-10 membered aryl or heteroaryl, —C₁-C₆ haloalkyl, —C₁-C₆ haloalkoxy, —C₁-C₆ deuterated alkyl, —C₁-C₆ deuterated alkoxy, —O-3-6 membered cycloalkyl or heterocycloalkyl, —C₁-C₆ alkyl OC₁-C₆ alkyl, —C₁-C₆ alkyl NHC₁-C₆ alkyl, —C₁-C₆ alkyl OH, —C₁-C₆ alkyl N (C₁-C₆ alkyl) (C₁-C₆ alkyl), and any two Rc can form 3-10 membered saturated or partially unsaturated carbocyclic or heterocyclic ring through carbon chain or heteroatom; R^{d} is independently selected from —C₁-C₆ alkyl, —C₁-C₆ alkyl OC₁-C₆ alkyl, —C₁-C₆ alkyl SC₁-C₆ alkyl, —C₁-C₆ alkyl SOC₁-C₆ alkyl, —C₁-C₆ alkyl SO₂C₁-C₆ alkyl, —COC₁-C₆ alkyl, —COOC₁-C₆ alkyl, —CONHC₁-C₆ alkyl, —CON (C₁-C₆ alkyl) (C₁-C₆ alkyl), 3-6 membered cycloalkyl or heterocycloalkyl, 5-10 membered aryl or heteroaryl, —C₁-C₆ haloalkyl, —C₁-C₆ haloalkoxy, —C₁-C₆ deuterated alkyl, —C₁-C₆ deuterated alkoxy-C₁-C₆ alkyl, —C₁-C₆ alkyl-O-3-6-membered cycloalkyl or heterocycloalkyl, —C₁-C₆ alkyl-NHC₁-C₆ alkyl, —C₁-C₆ alkyl OH, —C₁-C₆ alkyl N (C₁-C₆ alkyl) (C₁-C₆ alkyl), and the like.

In another some preferred embodiments, the compound has a structure shown in formula IV,

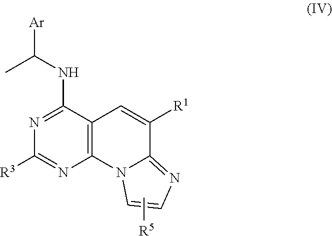

wherein, Ar, R¹, R³ and R⁵ are as defined above.

In another preferred embodiment, R¹ is 6-8 membered cycloalkyl or heterocycloalkyl, wherein the 6-8 membered cycloalkyl or heterocycloalkyl is optionally substituted by one or more R, and R is selected from: halogen (such as F), CN, OH, oxo, —C₁-C₃ alkyl (such as methyl, ethyl, propyl, isopropyl), —C₁-C₃ alkoxy (such as methoxy), —C(═O)C₁-C₃ alkyl, —C₁-C₆ alkyl 3-6 membered cycloalkyl, —C₁-C₆ alkyl 3-6 membered heterocycloalkyl, C(═O)(3-6 membered heterocyclyl) C₁-C₃ alkyl (such as

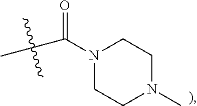 [SMILES: *C(=O)N1CCN(C)CC1]

C(═O) amino C₁-C₆ dialkyl OH (e.g.

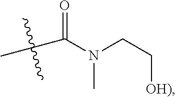

3-6 membered cycloalkyl or heterocycloalkyl (Example 71).

In another some preferred embodiments, R¹ is selected from

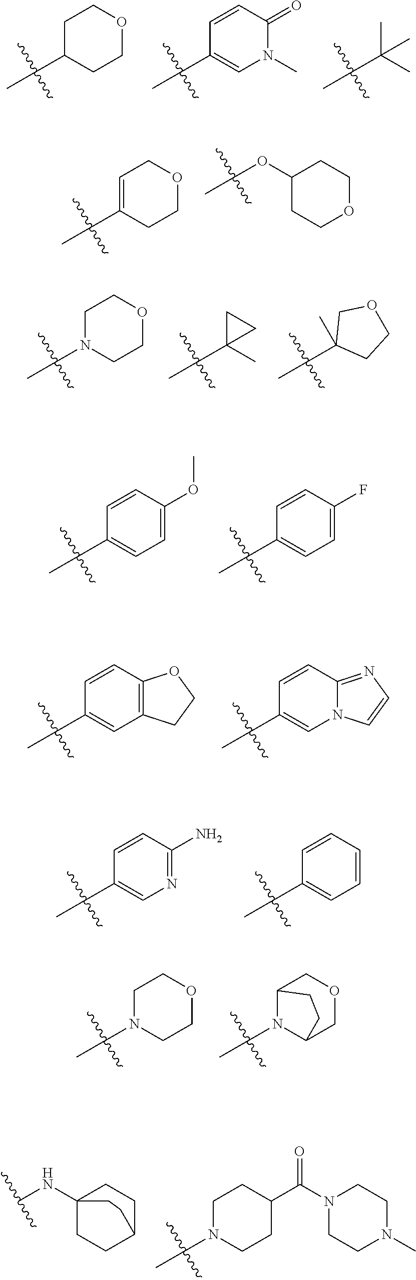

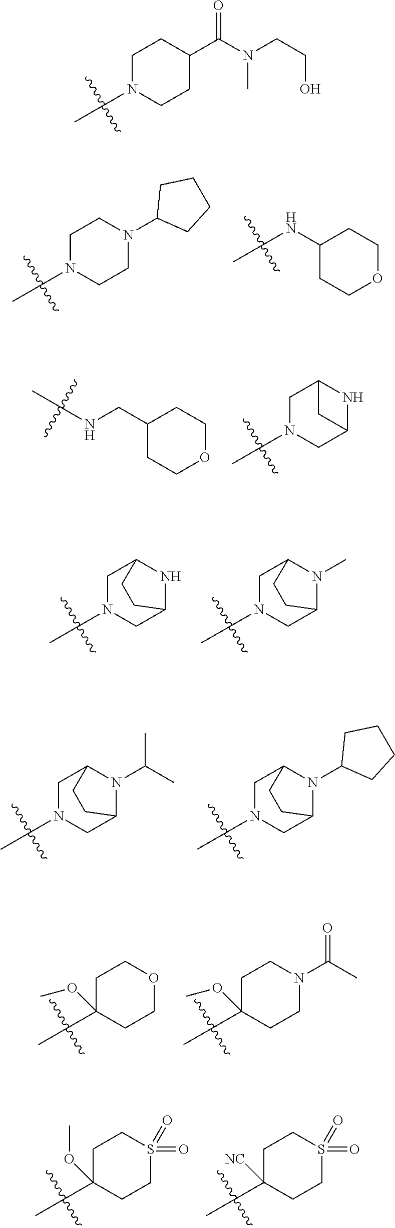

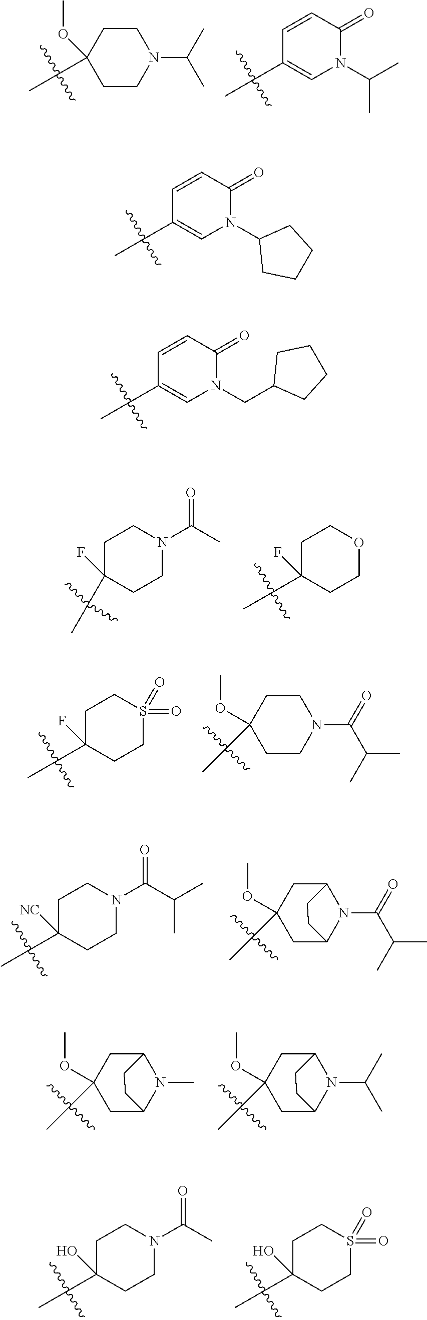

In another preferred embodiment, the substituted substituent in the substituted amido, substituted sulfonamido, substituted 5-12 membered aryl or heteroaryl is selected from C₁-C₃ alkyl, C₁-C₃ alkoxy, 3-6 membered cycloalkyl or heterocycloalkyl, C₁-C₁₀ dialkylaminoalkyl, halogen, and the like.

In another some preferred embodiments,

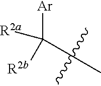

is selected from,

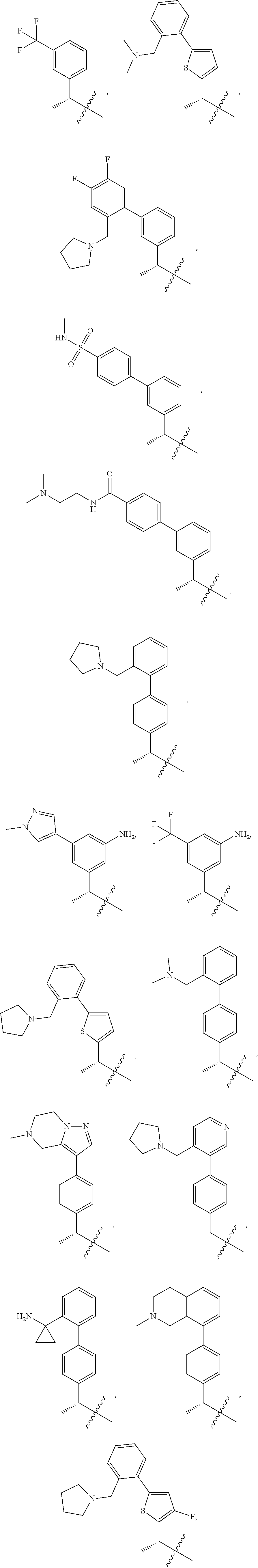

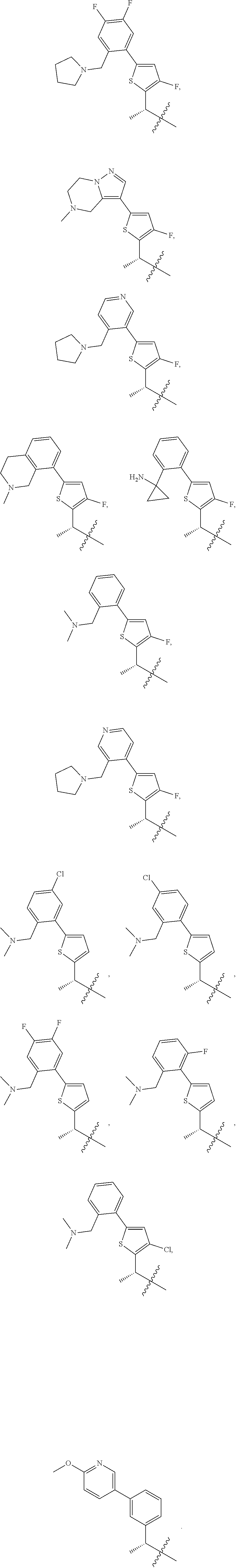

In another preferred embodiment, Ar, R¹, R^{2a}, R^{2b}, R³, R⁴, R⁶, Y, Z, Ar¹, R^{c} and Rd are the corresponding groups in each specific compound in the examples, respectively. Rc is alkyl or aryl, and the like; the other groups are as described above; in another some preferred embodiments, the compound includes, but is not limited to, the following structures:

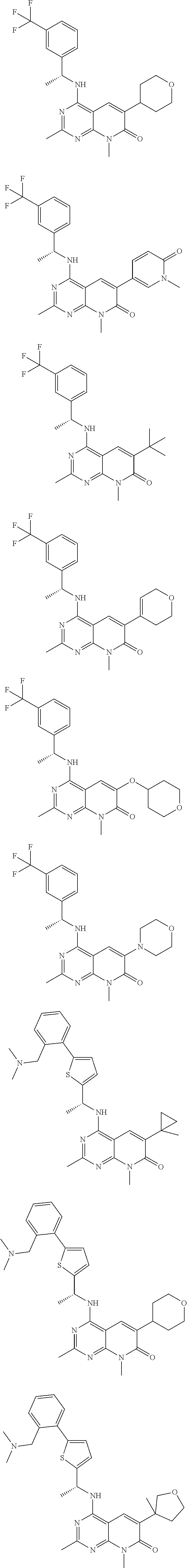

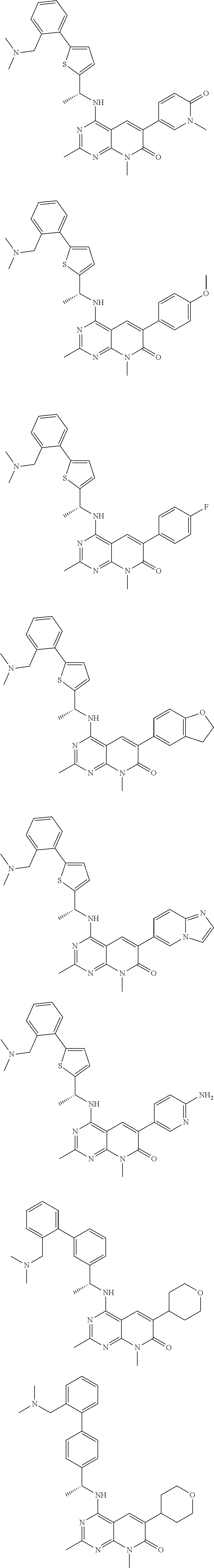

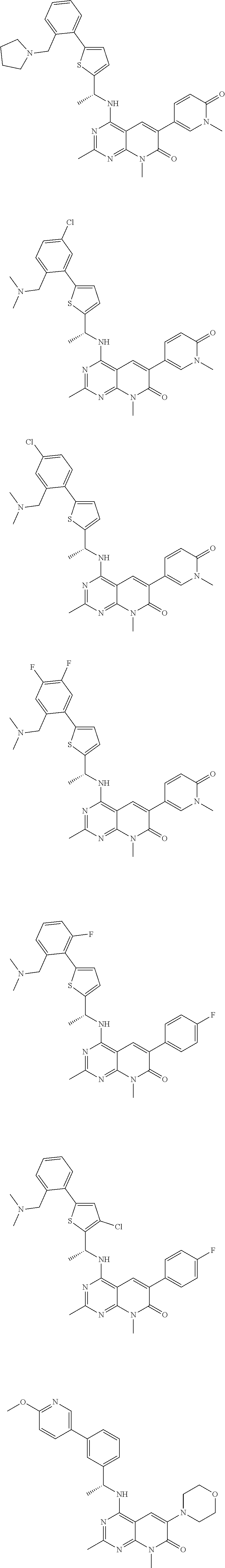

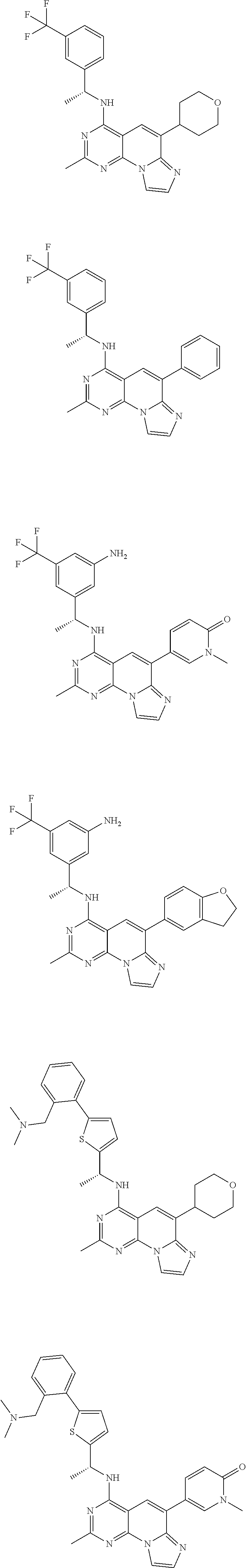

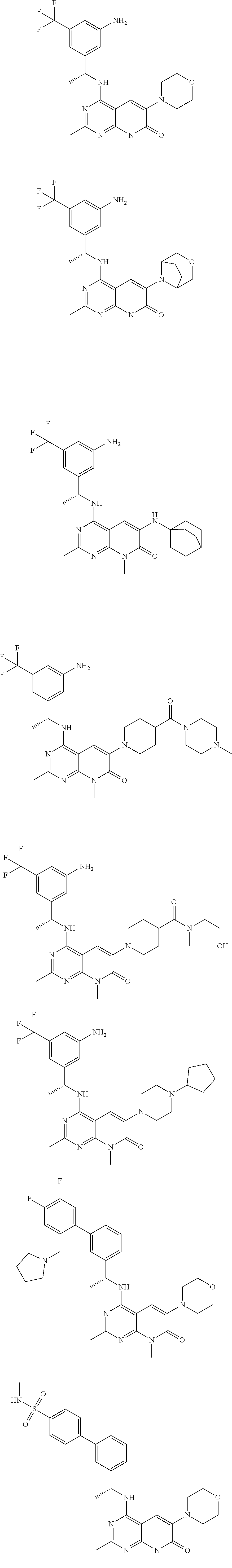

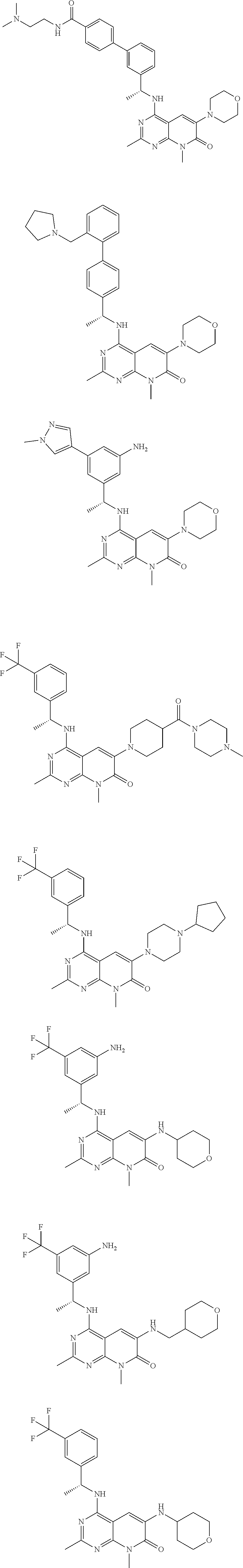

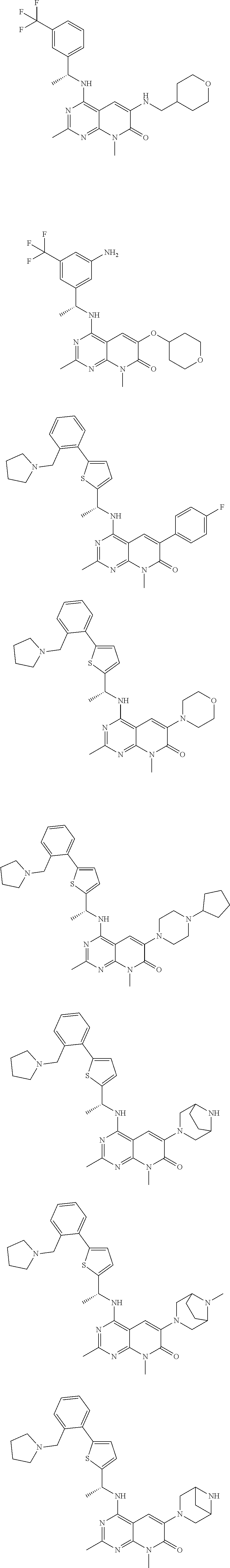

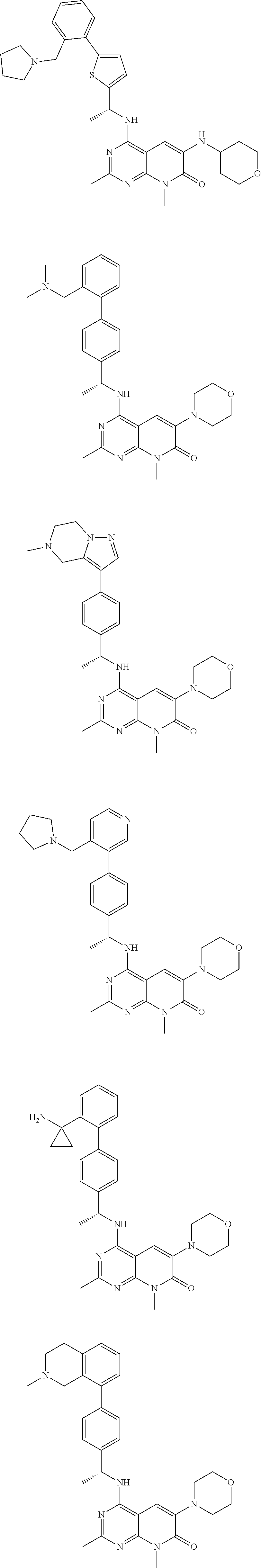 [SMILES: Cc1nc(N[C@H](C)c2ccc(-c3ccccc3CN3CCCC3)s2)c2cc(NC3CCOCC3)c(=O)n(C)c2n1 ; Cc1nc(N[C@H](C)c2ccc(-c3ccccc3CN(C)C)cc2)c2cc(N3CCOCC3)c(=O)n(C)c2n1 ; Cc1nc(N[C@H](C)c2ccc(-c3cnn4c3CN(C)CC4)cc2)c2cc(N3CCOCC3)c(=O)n(C)c2n1 ; Cc1nc(N[C@H](C)c2ccc(-c3cnccc3CN3CCCC3)cc2)c2cc(N3CCOCC3)c(=O)n(C)c2n1 ; Cc1nc(N[C@H](C)c2ccc(-c3ccccc3C3(N)CC3)cc2)c2cc(N3CCOCC3)c(=O)n(C)c2n1 ; Cc1nc(N[C@H](C)c2ccc(-c3cccc4c3CN(C)CC4)cc2)c2cc(N3CCOCC3)c(=O)n(C)c2n1]

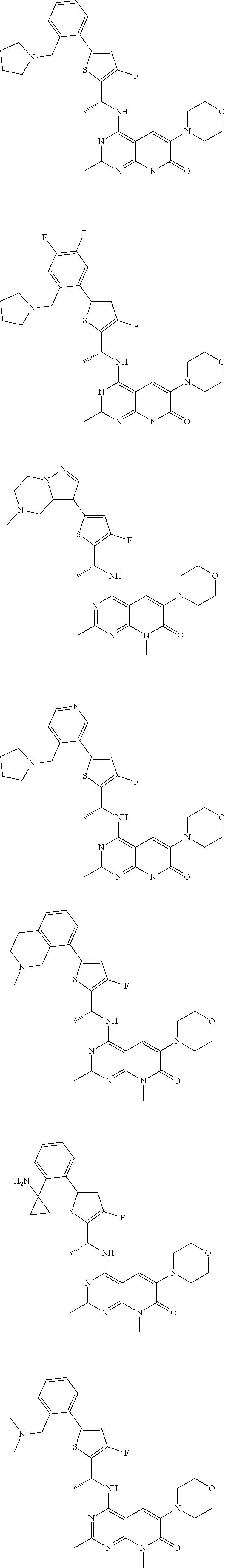

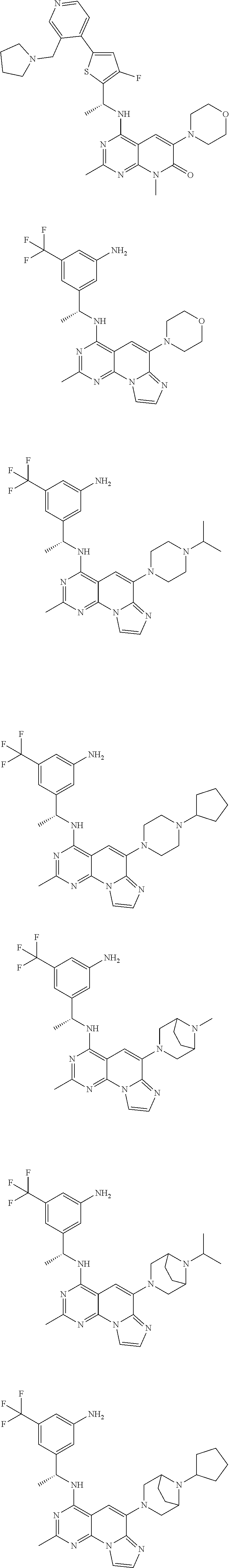

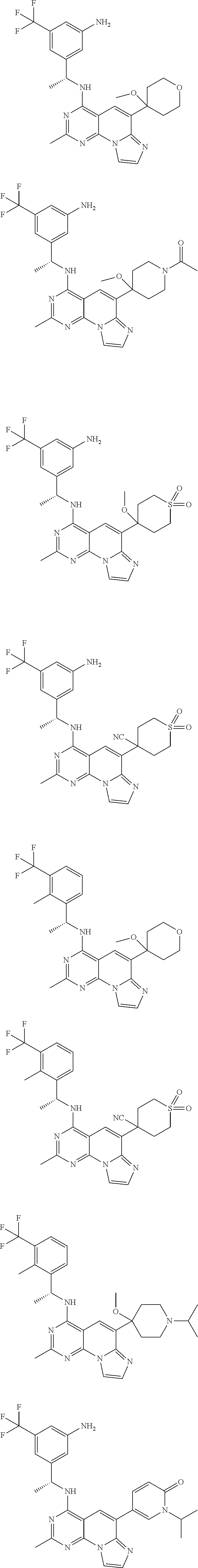

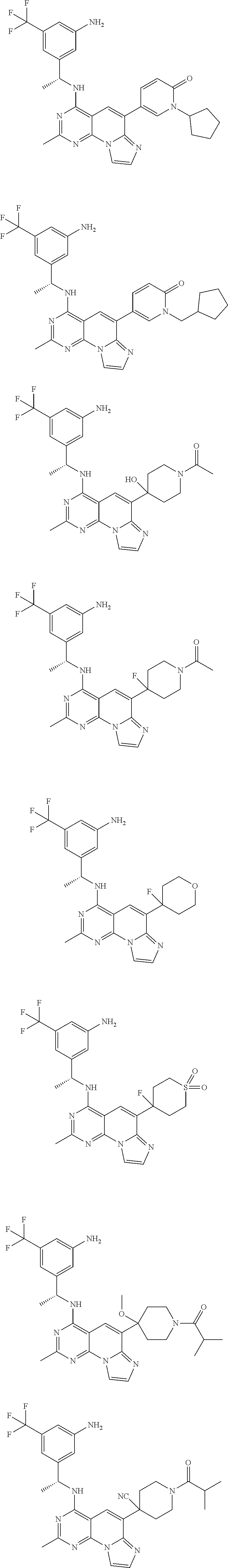

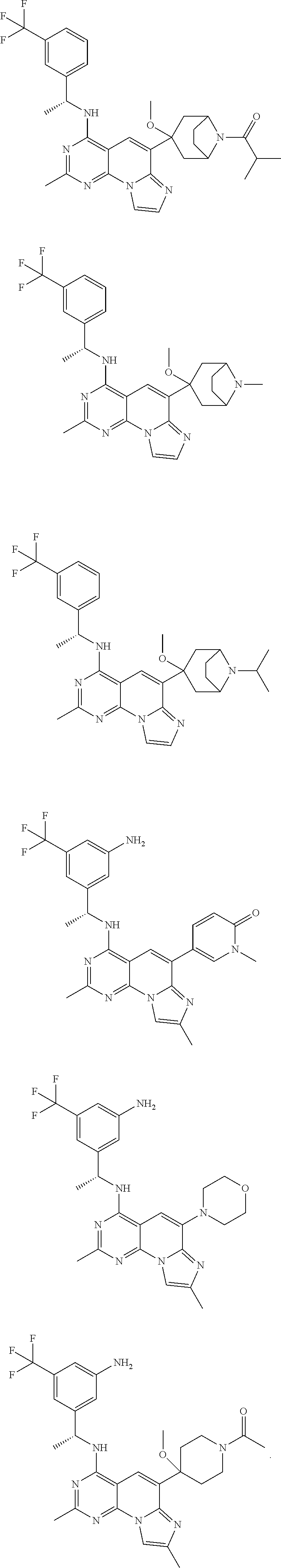 [SMILES: COC1(c2cc3c(N[C@H](C)c4cccc(C(F)(F)F)c4)nc(C)nc3n3ccnc23)CC2CCC(C1)N2C(=O)C(C)C ; COC1(c2cc3c(N[C@H](C)c4cccc(C(F)(F)F)c4)nc(C)nc3n3ccnc23)CC2CCC(C1)N2C ; COC1(c2cc3c(N[C@H](C)c4cccc(C(F)(F)F)c4)nc(C)nc3n3ccnc23)CC2CCC(C1)N2C(C)C ; Cc1cn2c(n1)c(-c1ccc(=O)n(C)c1)cc1c(N[C@H](C)c3cc(N)cc(C(F)(F)F)c3)nc(C)nc12 ; Cc1cn2c(n1)c(N1CCOCC1)cc1c(N[C@H](C)c3cc(N)cc(C(F)(F)F)c3)nc(C)nc12 ; COC1(c2cc3c(N[C@H](C)c4cc(N)cc(C(F)(F)F)c4)nc(C)nc3n3cc(C)nc23)CCN(C(C)=O)CC1]

In another preferred embodiment, the compound is selected from the compounds shown in the Examples. A method-one for preparing a compound of formula I, characterized in that the method comprises steps a, b:
a) subjecting the compound of general formula (A) and the compound of general formula (B) to a ring-closing reaction under the catalysis of a base, thereby forming the intermediate (C) compound;
b) subjecting the intermediate (C) and the compound of general formula (D) to a substitution reaction under the catalysis of a base, thereby generating the general formula (I).

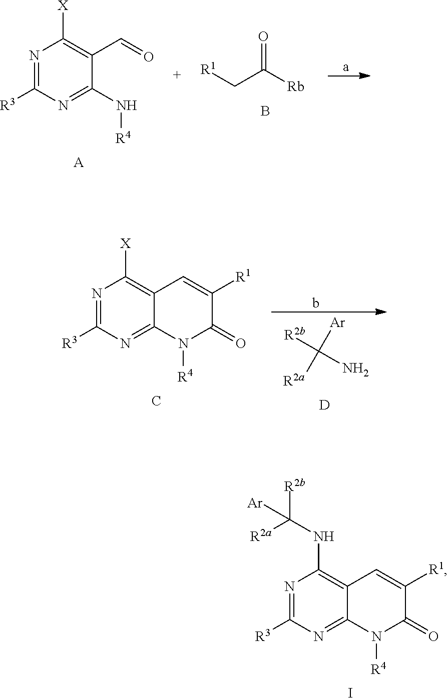

X is halogen; Rb is halogen or alkoxy, etc.; the other groups are as described above; A method-two for preparing a compound of formula I, characterized in that the method comprises steps c-f:
c) subjecting the compound of general formula (A), the compound of general formula (E) and p-methoxybenzaldehyde to a ring-closing reaction under the catalysis of a base or acid, thereby forming the intermediate (F) compound;
d) reacting the intermediate (F) with nitrite (or alkyl ester) and halogeno salt or (acid) and the like, thereby converting amino into the intermediate of general formula (G);
e) subjecting the intermediate compound of general formula (G) and the compound of general formula (D) to a substitution reaction under the catalysis of a base, thereby generating the intermediate compound of general formula (H);
f) coupling the intermediate compound of general formula (H) with various boronic acids (or esters), tin reagents, various substituted primary or secondary amines, or substituted alcohols under the catalysis of transition metal complexes, thereby generating general formula (I).

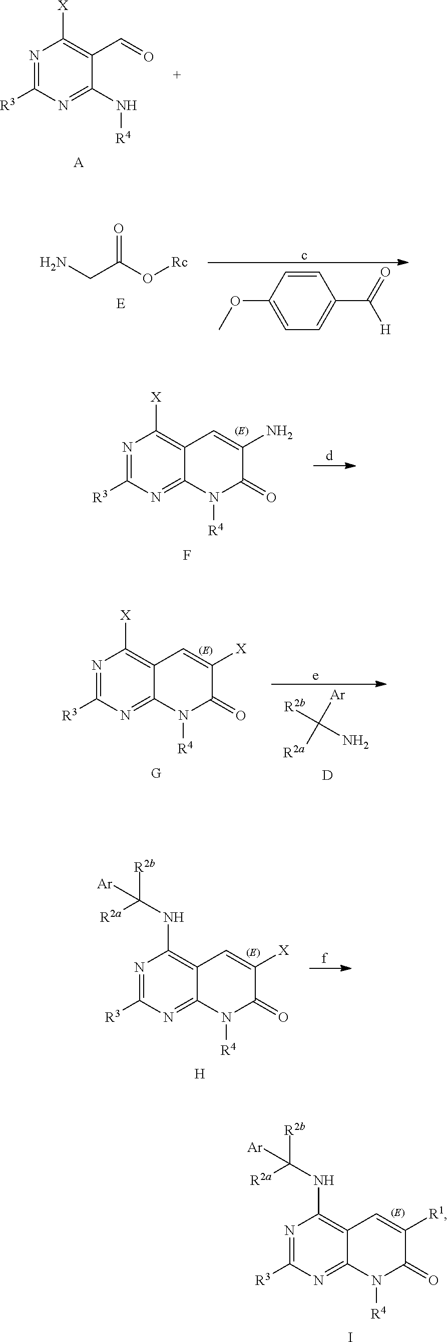

Rc is alkyl or aryl, and the like; the other groups are as described above;

In another aspect, it provides a method-three for preparing a compound of formula (I), the method comprises steps g-i:

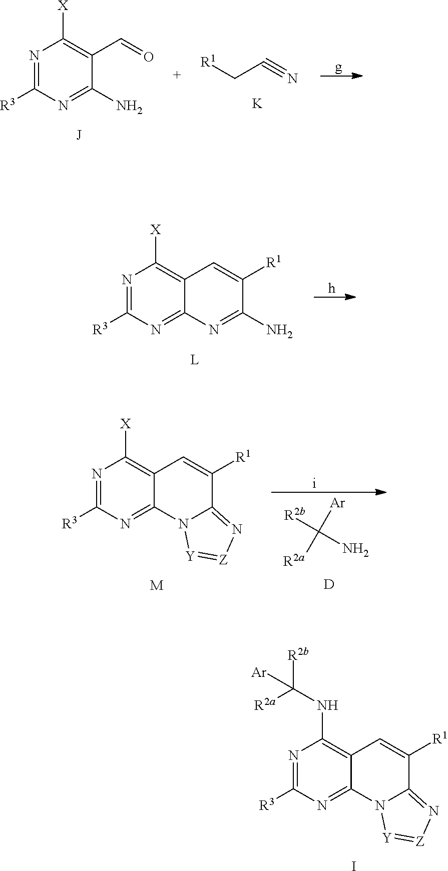

g) subjecting the compound of general formula (J) and the compound of general formula (K) to a ring-closing reaction under the catalysis of a base, thereby forming the intermediate (L) compound;
h) reacting the intermediate (L) with various halogenated ketones, halogenated aldehydes, substituted carboxylic acids, substituted acid chlorides and other raw materials or their equivalents under the catalysis of a base, thereby obtaining the intermediate (M); alternatively, intermediate (M) is synthesized according to the method of step a of the compound of general formula (I) in patent CN 110857300 A.
(i) subjecting the intermediate compound of general formula (M) to a substitution reaction with the compound of general formula (D) under the catalysis of a base, thereby generating the compound of general formula (I).

Preferably, each of the steps is performed in respective solvents, and the solvents are selected from the group consisting of water, methanol, ethanol, isopropanol, butanol, ethylene glycol, ethylene glycol methyl ether, N-methylpyrrolidone, dimethyl sulfoxide, tetrahydrofuran, toluene, dichloromethane, 1,2-dichloroethane, acetonitrile, N,N-dimethylformamide, N,N-dimethylacetamide, dioxane, or a combination thereof.

Preferably, the inorganic base is selected from the group consisting of sodium hydride, potassium hydroxide, sodium acetate, potassium acetate, potassium tert-butoxide, sodium tert-butoxide, potassium fluoride, cesium fluoride, potassium phosphate, potassium carbonate, potassium bicarbonate, sodium carbonate, sodium bicarbonate, or a combination thereof; the organic base is selected from the group consisting of pyridine, triethylamine, N,N-diisopropylethylamine, 1,8-diazabicyclo [5.4.0] undec-7-ene (DBU), lithium hexamethyldisilyl, sodium hexamethyldisilyl, dimethyl pyridine, or a combination thereof.

Preferably, the transition metal catalyst is selected from the group consisting of tris (dibenzylideneacetone) dipalladium (Pd₂(dba)₃), tetrakis (triphenylphosphine) palladium (Pd(PPh₃)₄), palladium acetate, palladium chloride, dichlorobis (triphenylphosphine) palladium, palladium trifluoroacetate, palladium triphenylphosphonoacetate, [1,1′-bis (diphenylphosphino) ferrocene] palladium dichloride, bis (triphenylmethylphosphine) palladium dichloride, 1,2-bis (diphenylphosphino) ethane palladium dichloride, or a combination thereof; the catalyst ligand is selected from the group consisting of tri-tert-butylphosphine, tri-tert-butylphosphine tetrafluoroborate, tri-n-butylphosphine, triphenylphosphine, tris-p-phenylmethylphosphine, tricyclohexylphosphine, tris(o-phenylmethyl)phosphine, or a combination thereof.

Preferably, the nitrite (or alkyl ester) is selected from the group consisting of sodium nitrite, potassium nitrite, isopropyl nitrate, isoamyl nitrite, tert-butyl nitrite, n-butyl nitrite, isobutyl nitrite, methyl nitrite, ethyl nitrite and the like, or a combination thereof.

Preferably, the halogeno salt is selected from the group consisting of potassium iodide, sodium iodide, cuprous iodide, cuprous bromide, cupric bromide, cupric chloride, cuprous chloride, and the like, or a combination thereof.

Preferably, the acid is selected from the group consisting of hydrochloric acid, hydrofluoric acid, hydrobromic acid, sulfuric acid, phosphoric acid, methanesulfonic acid, toluenesulfonic acid, trifluoroacetic acid, formic acid, acetic acid, trifluoromethanesulfonic acid, or a combination thereof.

Another object of the present invention is to provide a drug and a composition thereof for treating or preventing tumors. The technical solution to achieve the above object is as follows:
a pharmaceutical composition for treating tumors consisting of the pyrimido-fused-ring compound represented by above general formula (I), or a pharmaceutically acceptable salt thereof, or an enantiomer, a diastereomer, a tautomer, a torsional isomer, a solvate, a polymorph or a prodrug thereof, and a pharmaceutically acceptable carrier.

Another object of the present invention is to provide a use of the above compound. The technical solution to achieve the above object is as follows:

The pyrimido-fused-ring compound of formula (I), or a pharmaceutically acceptable salt thereof, or an enantiomer, a diastereomer, a tautomer, a torsional isomer, a solvate, a polymorph or a prodrug thereof is used for preparing a drug for treating a disease associated with mutation, activity or expression level of Ras, especially a drug for treating a tumor. The tumor is independently selected from non-small cell lung cancer, small cell lung cancer, lung adenocarcinoma, lung squamous cell carcinoma, breast cancer, prostate cancer, liver cancer, skin cancer, gastric cancer, intestinal cancer, cholangiocarcinoma, brain cancer, leukemia, lymphoma, fibroma, sarcoma, basal cell carcinoma, glioma, renal cancer, melanoma, bone cancer, thyroid cancer, nasopharyngeal carcinoma, pancreatic cancer, and the like.

The invention relates to a compound having a structure of general formula (I), which can inhibit a variety of tumor cells, in particular, can efficiently kill tumors associated with abnormal Ras protein signaling pathways, and is a kind of therapeutic drug with anew mechanism of action.

It should be understood that, within the scope of the present invention, the above technical features of the present invention and the technical features specifically described in the following (such as in the examples) may be combined with each other to form new or preferred technical solutions. It will not be repeated herein for the sake of space.

### DETAILED DESCRIPTION OF THE INVENTION

After long-term and in-depth research, the inventors have prepared a class of pyrimido heterocyclic compounds with a novel structure shown in formula I, and found that they have good inhibitory activity on SOS1 protein, and the compounds have specific inhibitory effect on SOS1 protein at extremely low concentration (as low as less than 20 nM), and have excellent inhibitory activity on cell proliferation related to Ras pathway, therefore, it can be used to treat related diseases caused by RAS mutation or abnormal activity or expression, such as tumors.

Based on the above findings, the inventors have completed the present invention.

### Terms

Unless otherwise defined, all scientific and technical terms herein have the same meaning as commonly understood by one of skill in the art to which the claimed subject matter belongs.

Unless otherwise indicated, all patents, patent applications, publications cited in their entirety herein are incorporated by reference in their entirety.

It is to be understood that both the foregoing brief description and the following detailed description are exemplary and explanatory only and are not intended to limit the inventive subject matter in any way. In the present application, the use of the singular also includes the plural unless specifically stated otherwise. It must be noted that, as used in the specification and claims, the singular includes the plural of what is referred to unless the context clearly dictates otherwise. It should also be noted that the use of “or”, “alternatively” means “and/or” unless otherwise specified. Furthermore, the use of the term “comprising” as well as other forms such as “comprise”, “containing” and “including” is not intended to be limiting.

Definitions of standard chemical terms can be found in references including Carey and Sundberg “ADVANCED ORGANIC CHEMISTRY 4TH ED.” Vols. A (2000) and B (2001), Plenum Press, New York. Conventional methods within the scope of the art, such as mass spectrometry, NMR, IR and UV/VIS spectroscopy and pharmacological methods, are employed unless otherwise indicated. Unless specifically defined, the terms used herein in relation to analytical chemistry, organic synthetic chemistry and pharmaceutical and pharmaceutical chemistry are known in the art. Standard techniques can be used in chemical synthesis, chemical analysis, pharmaceutical preparation, preparation and delivery as well as treatment in patients. For example, the reaction and purification can be carried out by using the manufacturer's instructions for use of the kit, or by methods well known in the art or as described in the present invention. The above techniques and methods can generally be carried out according to conventional methods well known in the art and the description in various summary and more specific references cited and discussed in this specification. In the present specification, the group and its substituents can be selected by those skilled in the art to provide stable structural moieties and compounds.

When a substituent is described by a conventional chemical formula written from left to right, the substituent also includes a chemically equivalent substituent obtained when the structural formula is written from right to left. For example, —CH₂O— is equivalent to —OCH₂—.

The section titles used herein are for the purpose of organizing articles only and should not to be construed as limitation of the subject matter. All literatures or parts of the literatures cited in this application, including but not limited to patents, patent applications, articles, books, operating manuals and papers, are hereby incorporated by reference in their entirety.

Certain chemical groups defined herein are preceded by a simplified symbol to indicate the total number of carbon atoms present in the group. For example, C₁₋₆ alkyl refers to an alkyl as defined below having a total of 1 to 6 carbon atoms. The total number of carbon atoms in the simplified symbol does not include carbon that may be present in the substituents of the group.

In addition to the foregoing, when used in the specification and claims of the present application, the following terms have the meanings indicated below, unless otherwise specifically indicated.

In the present application, the term “halogen” refers to fluorine, chlorine, bromine or iodine; “hydroxy” refers to an —OH group; “hydroxyalkyl” refers to an alkyl as defined below substituted with a hydroxy (—OH) group; “carbonyl” refers to a —C(═O)— group; “nitro” refers to —NO₂; “cyano” refers to —CN; “amino” means —NH₂; “substituted amino” means an amino substituted with one or two alkyl, alkylcarbonyl, aralkyl, heteroaralkyl as defined below, e.g., monoalkylamino, dialkylamino, alkylamido, aralkylamino, heteroaralkylamino; “carboxy” means —COOH.

In the present application, as a group or part of another group (e.g., used in a group such as halogen-substituted alkyl and the like), the term “alkyl” means a straight or branched hydrocarbon chain radical consisting solely of carbon and hydrogen atoms, free of unsaturated bond, having, for example, from 1 to 12 (preferably from 1 to 8, more preferably from 1 to 6) carbon atoms, and attached to the rest of the molecule by a single bond. Examples of alkyl include, but are not limited to, methyl, ethyl, n-propyl, isopropyl, n-butyl, isobutyl, sec-butyl, tert-butyl, n-pentyl, 2-methylbutyl, 2,2-dimethylpropyl, n-hexyl, heptyl, 2-methylhexyl, 3-methylhexyl, octyl, nonyl, and decyl, and the like.

In the present application, the term “alkenyl”, as a group or part of another group, means a straight or branched hydrocarbon chain radical consisting only of carbon and hydrogen atoms, containing at least one double bond, having, for example, from 2 to 14 (preferably from 2 to 10, more preferably from 2 to 6) carbon atoms, attached to the rest of the molecule by a single bond, for example, but not limited to, ethenyl, propenyl, allyl, but-1-enyl, but-2-enyl, pent-1-enyl, pent-1,4-dienyl, and the like.

In the present application, the term “alkynyl”, as a group or part of another group, means a straight or branched hydrocarbon chain radical consisting solely of carbon and hydrogen atoms, containing at least one triple bond and optionally one or more double bonds, having, for example, from 2 to 14 (preferably from 2 to 10, more preferably from 2 to 6) carbon atoms, attached to the rest of the molecule by a single bond, for example, but not limited to, ethynyl, prop-1-ynyl, but-1-ynyl, pent-1-en-4-ynyl, and the like.

In the present application, as a group or part of another group, the term “cycloalkyl” means a stable non-aromatic monocyclic or polycyclic hydrocarbon group consisting solely of carbon and hydrogen atoms, which may include fused, bridged or spiro ring systems, having from 3 to 15 carbon atoms, preferably from 3 to 10 carbon atoms, more preferably from 3 to 8 carbon atoms, and it is saturated or unsaturated and can be attached to the rest of the molecule by a single bond via any suitable carbon atom. Unless specifically stated otherwise in the specification, carbon atoms in the cycloalkyl may optionally be oxidized. Examples of cycloalkyl include, but are not limited to, cyclopropyl, cyclobutyl, cyclopentyl, cyclopentenyl, cyclohexyl, cyclohexenyl, cyclohexadienyl, cycloheptyl, cyclooctyl, 1H-indenyl, 2,3-indanyl, 1,2,3,4-tetrahydro-naphthyl, 5,6,7,8-tetrahydro-naphthyl, 8,9-dihydro-7H-benzocycloheptene-6-yl, 6,7,8,9-tetrahydro-5H-benzocycloheptenyl, 5,6,7,8,9,10-hexahydro-benzocyclooctenyl, fluorenyl, bicyclo[2.2.1]heptyl, 7,7-dimethyl-bicyclo[2.2.1]heptyl, bicyclo[2.2.1]heptenyl, bicyclo[2.2.2]octyl, bicyclo[3.1.1]heptyl, bicyclo[3.2.1]octyl, bicyclo[2.2.2]octenyl, bicyclo[3.2.1]octenyl, adamantyl, octahydro-4,7-methylene-1H-indenyl and octahydro-2,5-methylene-cyclopentadienyl and the like. In the present invention, cycloalkyl and carbocyclyl are used interchangeably. In the present invention, 3-12 membered cycloalkyl and C₃-C₁₂ cycloalkyl are used interchangeably. Similarly, 3-6 membered cycloalkyl is used interchangeably with C₃-C₆ cycloalkyl.

In the present application, as a group or part of another group, the term “heterocyclyl (or heterocycloalkyl)” means a stable 3- to 20-membered non-aromatic cyclic group consisting of 2 to 14 carbon atoms and 1 to 6 heteroatoms selected from nitrogen, phosphorus, oxygen and sulfur.

Unless otherwise specifically indicated in the specification, a heterocyclyl may be a monocyclic, bicyclic, tricyclic or more cyclic ring system, which may include a fused ring system, a bridged ring system or a spiro ring system. In the heterocyclyl, the nitrogen, carbon or sulfur atom may optionally be oxidized and the nitrogen atom may optionally be quaternized. The heterocyclyl may be partially or fully saturated. The heterocyclyl may be attached to the remainder of the molecule via a carbon atom or a heteroatom by a single bond. In the heterocyclyl containing a fused ring, one or more of the rings may be an aryl or heteroaryl group as defined hereinafter, provided that the connection point to the rest of the molecule is a non-aromatic ring atom. For the purpose of the present invention, the heterocyclyl is preferably a stable 4 to 11 membered non-aromatic monocyclic, bicyclic, bridged or spiro group containing 1 to 3 heteroatoms selected from nitrogen, oxygen and sulfur; more preferably a stable 4- to 8-membered non-aromatic monocyclic, bicyclic, bridged or spiro group containing 1 to 3 heteroatoms selected from nitrogen, oxygen and sulfur. Examples of heterocyclyl include (but are not limited to) pyrrolidinyl, morpholinyl, piperazinyl, homopiperazinyl, piperidinyl, thiomorpholinyl, 2,7-diaza-spiro[3.5]nonane-7-yl, 2-oxa-6-aza-spiro[3.3]heptane-6-yl, 2,5-diaza-bicyclo[2.2.1]heptan-2-yl, azacyclobutanyl, pyranyl, tetrahydropyranyl, thiopyranyl, tetrahydrofuranyl, oxazinyl, dioxolanyl, tetrahydroisoquinolinyl, decahydroisoquinolinyl, imidazolinyl, imidazolidinyl, quinazinyl, thiazolidinyl, isothiazolidinyl, isoxazolidinyl, indolinyl, octahydroindolyl, octahydroisoindolyl, pyrrolidinyl, pyrazolidinyl, phthalimido and the like. In the present invention, heterocyclyl or heterocycloalkyl can be used interchangeably.

In the present invention, the spiro ring refers to a carbocyclic group or a heterocyclic group sharing one carbon atom, preferably is 5-11 membered, more preferably is 7-11 membered. Examples of spiro rings include, but are not limited to:

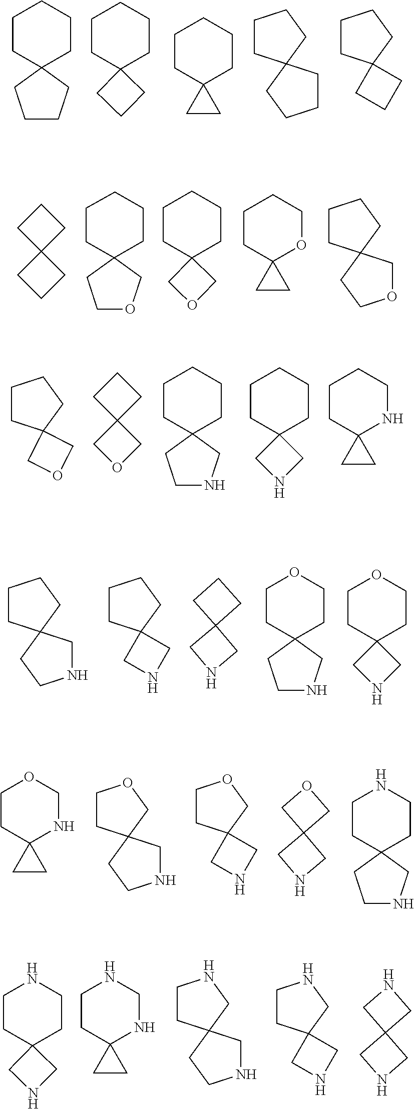

In the present invention, the fused ring refers to a carbocyclic or heterocyclic group sharing two adjacent carbon atoms, preferably is 4-10 membered, more preferably is 7-10 membered. Examples of fused rings include, but are not limited to:

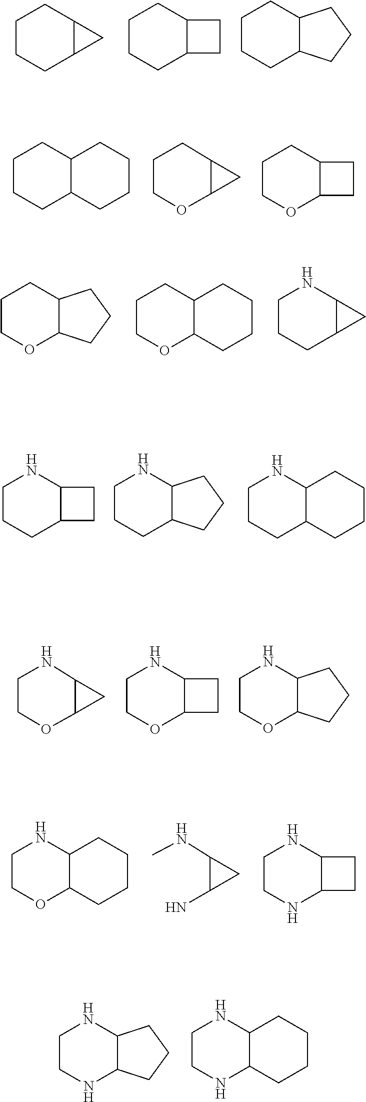

In the present invention, a bridged ring refers to a carbocyclic or heterocyclic group having a total of two non-adjacent carbon atoms, preferably is 7-8 membered. Examples of bridge rings include, but are not limited to:

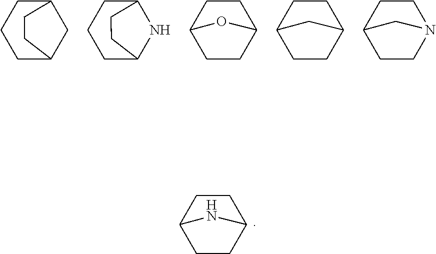

In the present invention, the “carbocycle or heteroatom-containing spiro ring/bridged ring/fused ring” includes the aforementioned spiro ring, bridged ring and fused ring, and the carbocycle or heteroatom-containing spiro ring/bridged ring/fused ring is preferably a 7-11 membered spiro ring, a 7-10 membered fused ring or a 7-8 membered bridged ring.

In the present application, as a group or part of another group, the term “aryl” means a conjugated hydrocarbon ring system radical having 6 to 18 carbon atoms, preferably having 6 to 10 carbon atoms. For the purpose of the present invention, an aryl may be a monocyclic, bicyclic, tricyclic or more cyclic ring system, and may also be fused to a cycloalkyl or heterocyclyl as defined above, provided that the aryl is connected to the rest of the molecule via atoms on the aromatic ring by a single bond. Examples of aryl include (but are not limited to) phenyl, naphthyl, anthracenyl, phenanthryl, fluorenyl, 2,3-dihydro-1H-isoindolyl, 2-benzoxazolinone, 2H-1,4-benzoxazine-3(4H)-keto-7-yl and the like.

In the present application, the term “arylalkyl” refers to an alkyl as defined above substituted with an aryl as defined above.

In the present application, as a group or part of another group, the term “heteroaryl” means a 5- to 16-membered conjugated ring system group having 1 to 15 carbon atoms (preferably 1 to 10 carbon atoms) and 1 to 6 heteroatoms selected from nitrogen, oxygen and sulfur in the ring.

Unless otherwise specifically indicated in the specification, a heteroaryl may be a monocyclic, bicyclic, tricyclic or more cyclic ring system, and may also be fused to a cycloalkyl or heterocyclyl as defined above, provided that the heteroaryl is connected to the remainder of the molecule via an atom on the aromatic ring by a single bond. In the heteroaryl, the nitrogen, carbon or sulfur atom can be optionally oxidized and the nitrogen atom can optionally be quaternized. For the purpose of the present invention, the heteroaryl is preferably a stable 5- to 12-membered aromatic group containing 1 to 5 heteroatoms selected from nitrogen, oxygen and sulfur, more preferably a stable 5- to 10-membered aromatic group containing 1 to 4 heteroatoms selected from nitrogen, oxygen and sulfur or a 5- to 6-membered aromatic group containing 1 to 3 heteroatoms selected from nitrogen, oxygen and sulfur. Examples of heteroaryl include (but are not limited to) thienyl, imidazolyl, pyrazolyl, thiazolyl, oxazolyl, oxadiazolyl, isoxazolyl, pyridyl, pyrimidinyl, pyrazinyl, pyridazinyl, benzimidazolyl, benzopyrazolyl, indolyl, furyl, pyrrolyl, triazolyl, tetrazolyl, triazinyl, indolizinyl, isoindolyl, indazolyl, isoindazolyl, purinyl, quinolyl, isoquinolyl, diaza naphthyl, naphthyridinyl, quinoxalinyl, pteridyl, carbazolyl, carbolinyl, phenanthridinyl, phenanthrolinyl, acridinyl, phenazinyl, isothiazolyl, benzothiazolyl, benzothienyl, oxtriazolyl, cinnolinyl, quinazolinyl, phenylthio, indolizinyl, o-phenanthrolinyl, isoxazolyl, phenoxazinyl, phenothiazinyl, 4,5,6,7-tetrahydrobenzo[b]thienyl, naphthopyridyl, [1,2,4]triazolo[4, 3-b]pyridazine, [1,2,4]triazolo[4,3-a]pyrazine, [1,2,4]triazolo[4,3-c]pyrimidine, [1,2,4]triazolo[4,3-a]pyridine, imidazo[1,2-a]pyridine, imidazo[1,2-b]pyridazine, imidazo[1,2-a]pyrazine and the like.

In the present application, the term “heteroarylalkyl” refers to an alkyl as defined above substituted with a heteroaryl as defined above.

In the present application, “optional” or “optionally” means that the subsequently described event or condition may or may not occur, and that the description includes both occurrences and non-occurrences of the event or condition. For example, “optionally substituted aryl” means that the aryl is substituted or unsubstituted, and the description includes both the substituted aryl and the unsubstituted aryl.

The terms “moiety”, “structural moiety”, “chemical moiety”, “group” and “chemical group” as used herein refer to a particular fragment or functional group in a molecule. A chemical moiety is generally considered to be a chemical entity that is embedded or attached to a molecule.

“Stereoisomer” refers to a compound composed of same atoms, bonded by same bonds, but having a different three-dimensional structure. The invention will cover various stereoisomers and mixtures thereof.

When the compound of the present invention contains an olefinic double bond, the compounds of the present invention are intended to include E- and Z-geometric isomers unless otherwise stated.

“Tautomer” refers to an isomer formed by the transfer of a proton from one atom of a molecule to another atom of the same molecule. All tautomeric forms of the compounds of the invention will also be included in the scope of the invention.

The compounds of the present invention or pharmaceutically acceptable salts thereof may contain one or more chiral carbon atoms, thereby forming enantiomers, diastereomers, and other stereoisomeric forms. Each chiral carbon atom can be defined as (R)- or (S)-based on stereochemistry. The invention is intended to include all possible isomers, as well as racemate and optically pure forms thereof. Racemates, diastereomers or enantiomers may be used as starting materials or intermediates for the preparation of the compounds of the invention. Optically active isomers can be prepared using chiral synthons or chiral reagents, or resolved using conventional techniques, such as by crystallization and chiral chromatography.

Conventional techniques for the preparation/separation of individual isomers include chiral synthesis from appropriate optically pure precursors, or resolution of the racemate (or racemate of a salt or derivative) using, for example, chiral high performance liquid chromatography, see, for example, Gerald Gubitz and Martin G. Schmid (Eds.), Chiral Separations, Methods and Protocols, Methods in Molecular Biology, Vol. 243, 2004; A. M. Stalcup, Chiral Separations, Annu. Rev. Anal. Chem. 3:341-63, 2010; Fumiss et al. (eds.), VOGEL'S ENCYCLOPEDIA OF PRACTICAL ORGANIC CHEMISTRY 5.sup. TH ED., Longman Scientific and Technical Ltd., Essex, 1991, 809-816; Heller, Acc. Chem. Res. 1990, 23, 128.

In the present application, the term “pharmaceutically acceptable salt” includes pharmaceutically acceptable acid addition salts and pharmaceutically acceptable base addition salts.

“Pharmaceutically acceptable acid addition salt” means a salt formed with an inorganic acid or organic acid which retains the bioavailability of the free base without other side effects. Inorganic acid salts include, but are not limited to, hydrochlorides, hydrobromides, sulfates, nitrates, phosphates, and the like; organic acid salts include, but are not limited to, formate, acetate, 2,2-dichloroacetate, trifluoroacetate, propionate, hexanoate, octoate, decanoate, undecylenate, glycolate, gluconate, lactate, sebacate, adipate, glutarate, malonates, oxalates, maleates, succinates, fumarates, tartrates, citrates, palmitates, stearates, oleates, cinnamate, laurate, malate, glutamate, pyroglutamate, aspartate, benzoate, methanesulfonate, besylate, p-toluenesulfonate, alginate, ascorbate, salicylate, 4-aminosalicylate, naphthalene disulfonate, and the like. These salts can be prepared by methods known in the art.

“Pharmaceutically acceptable base addition salt” means a salt formed with an inorganic base or organic base that is capable of maintaining the bioavailability of the free acid without other side effects. Salts derived from inorganic bases include, but are not limited to, sodium salts, potassium salts, lithium salts, ammonium salts, calcium salts, magnesium salts, iron salts, zinc salts, copper salts, manganese salts, aluminum salts, and the like. Preferred inorganic salts are ammonium, sodium, potassium, calcium and magnesium salts. Salts derived from organic bases include, but are not limited to, the following salts: primary amines, secondary amines and tertiary amines, substituted amines, including naturally substituted amines, cyclic amines, and basic ion exchange resins, for example, ammonia, isopropylamine, trimethylamine, diethylamine, triethylamine, tripropylamine, ethanolamine, diethanolamine, triethanolamine, dimethylethanolamine, 2-dimethylaminoethanol, 2-diethylaminoethanol, bicyclo hexylamine, lysine, arginine, histidine, caffeine, procaine, choline, betaine, ethylenediamine, glucosamine, methylglucosamine, theobromine, purine, piperazine, piperidine, N-ethylpiperidine, polyamine resin, and the like. Preferred organic bases include isopropylamine, diethylamine, ethanolamine, trimethylamine, dicyclohexylamine, choline and caffeine. These salts can be prepared by methods known in the art.

“Polymorph” refers to different solid crystalline phases of certain compounds of the present invention in the solid state due to the presence of two or more different molecular arrangements.

Certain compounds of the present invention may exist in more than one crystalline form, and the present invention is intended to include various crystalline forms and mixtures thereof.

Generally, crystallization will result in solvates of the compounds of the invention. As used herein, the term “solvate” refers to an aggregate comprising one or more molecules of a compound of the invention and one or more molecules of a solvent. The solvent may be water, in which case the solvate is a hydrate. Alternatively, the solvent may be an organic solvent. Thus, the compounds of the invention may exist as hydrates, including monohydrates, dihydrates, hemihydrates, sesquihydrates, trihydrates, tetrahydrates, and the like, as well as corresponding solvated forms. The compounds of the invention may form true solvates, but in some cases may retain only indefinite water or a mixture of water plus some indefinite solvent. The compounds of the present invention may be reacted in a solvent or precipitated or crystallized from a solvent. Solvates of the compounds of the present invention are also included in the scope of the present invention.

The present invention also includes prodrugs of the above compounds. In the present application, the term “prodrug” denotes a compound that can be converted to the biologically active compound of the present invention under physiological conditions or by solvolysis. Thus, the term “prodrug” refers to a pharmaceutically acceptable metabolic precursor of a compound of the invention. A prodrug may be inactive when administered to an individual in need thereof, but is converted in vivo to an active compound of the invention. Prodrugs are typically rapidly transformed in vivo to yield the parent compound of the invention, for example by hydrolysis in blood. Prodrug compounds typically provide advantages of solubility, tissue compatibility, or sustained release in mammalian organisms. Prodrugs include known amino protecting groups and carboxyl protecting groups. For specific preparation method for prodrugs, reference may be made to Saulnier, M. G., et al., Bioorg. Med. Chem. Lett. 1994, 4, 1985-1990; Greenwald, R. B., et al., J. Med. Chem. 2000, 43, 475.

In the present application, a “pharmaceutical composition” refers to a formulation of a compound of the invention in combination with a medium generally accepted in the art for delivery of a biologically active compound to a mammal (e.g., a human). The medium includes a pharmaceutically acceptable carrier. The purpose of the pharmaceutical composition is to promote the administration of the organism, thereby facilitating the absorption of the active ingredient and thereby exerting biological activity.

The term “pharmaceutically acceptable” as used herein refers to a substance (such as a carrier or diluent) that does not affect the biological activity or properties of the compound of the invention, and is relatively non-toxic, i.e, the substance can be administered to an individual without causing undesirable biological reaction or interacting with any of the components contained in the composition in an undesirable manner.

In the present application, “pharmaceutically acceptable carrier” include, but are not limited to, any adjuvants, carriers, excipients, glidants, sweeteners, diluents, preservatives, dyes/colorants, flavoring agents, surfactants, wetting agents, dispersing agents, suspending agents, stabilizers, isotonic agents, solvents or emulsifiers, that are approved by the relevant government authorities for acceptable use for humans or domestic animals.

The “tumors” and “diseases related to abnormal cell proliferation” of the present invention include but are not limited to leukemia, gastrointestinal stromal tumor, histiocytic lymphoma, non-small cell lung cancer, small cell lung cancer, pancreatic cancer, lung squamous cell carcinoma, lung adenocarcinoma, breast cancer, prostate cancer, liver cancer, skin cancer, epithelial cell carcinoma, cervical cancer, ovarian cancer, intestinal cancer, nasopharyngeal cancer, brain cancer, bone cancer, esophageal cancer, melanin tumor, kidney cancer, oral cancer and the like.

The terms “preventive”, “preventing” and “prevent” as used herein include reducing the possibility of the occurrence or deterioration of a disease or condition in a patient.

The term “treatment” and other similar synonyms as used herein includes the following meanings:
(i) preventing the occurrence of a disease or condition in a mammal, especially when such mammal is predisposed to suffer from the disease or condition but has not been diagnosed as having the disease or condition;
(ii) inhibiting a disease or condition, i.e. suppressing its development;
(iii) alleviating the disease or condition, i.e., causing the state of the disease or condition to subside; or
(iv) alleviating the symptoms caused by the disease or condition.

The term “effective amount”, “therapeutically effective amount” or “pharmaceutically effective amount” as used herein refers to a amount of at least one agent or compound that is sufficient to alleviate one or more symptoms of the disease or condition being treated to some extent after being administered. The result can be a reduction and/or alleviation of signs, symptoms or causes, or any other desired change in the biological system. For example, an “effective amount” for treatment is an amount of the composition comprising the compound disclosed herein that is required to provide a significant clinical condition relief effect. An effective amount suitable for any individual case can be determined using techniques such as dose escalation testing.

As used herein, the terms “administration”, “administered”, “administering” and the like refers to a method capable of delivering a compound or composition to a desired site for biological action. These methods include, but are not limited to, oral routes, duodenal routes, parenteral injection (including intravenous, subcutaneous, intraperitoneal, intramuscular, intraarterial injection or infusion), topical administration, and rectal administration. The skilled person in the art is well known for the administration techniques of the compounds and methods described herein, such as those discussed in Goodman and Gilman, The Pharmacological Basis of Therapeutics, current ed.; Pergamon; and Remington's, Pharmaceutical Sciences (current edition), Mack Publishing Co., Easton, Pa. In a preferred embodiment, the compounds and compositions discussed herein are administered orally.

As used herein, the terms “pharmaceutical combination”, “drug combination”, “combined medication”, “applying other treatments”, “administering other therapeutic agents” and the like mean a pharmaceutical treatment obtained by mixing or combining more than one active ingredient, including both fixed and unfixed combinations of active ingredients. The term “fixed combination” refers to the simultaneous administration of at least one compound described herein and at least one synergistic agent to a patient in the form of a single entity or a single dosage form. The term “unfixed combination” refers to the simultaneous administration, combination or sequential administration in variable intervals of at least one of the compounds described herein and at least one synergistic agent to the patient in the form of separate entities. These are also applied to cocktail therapy, for example the administration of three or more active ingredients.

It will also be understood by those skilled in the art that, in the methods described below, functional groups of the intermediate compound may need to be protected by a suitable protecting group. Such functional groups include a hydroxyl group, an amino group, a thiol group and a carboxylic acid. Suitable hydroxyl protecting groups include trialkylsilyl or diarylalkylsilyl (e.g., tert-butyldimethylsilyl, tert-butyldiphenylsilyl, or trimethylsilyl), tetrahydropyranyl, benzyl, and the like. Suitable protecting groups for amino, guanyl and guanidyl include t-butoxycarbonyl, benzyloxycarbonyl and the like. Suitable thiol protecting groups include —C(O)—R″(wherein R″ is alkyl, aryl, or aralkyl), p-methoxybenzyl, trityl, and the like. Suitable carboxy protecting groups include alkyl, aryl or aralkyl esters.

Protecting groups can be introduced and removed according to standard techniques known to those skilled in the art and as described herein. The use of protecting groups is described in detail in Greene, T. W. and P. G. M. Wuts, Protective Groups in Organic Synthesis, (1999), 4th Ed., Wiley. The protecting group can also be a polymeric resin.

The present invention is further illustrated below with reference to specific examples. It should be understood that these examples are merely illustrative of the invention and are not intended to limit the scope of the invention. The experimental methods with no specific conditions described in the following examples are generally performed under the conventional conditions, or according to the manufacture's instructions. Unless otherwise stated, percentages and parts are percentages by weight and parts by weight.

### Example General Preparation Method I

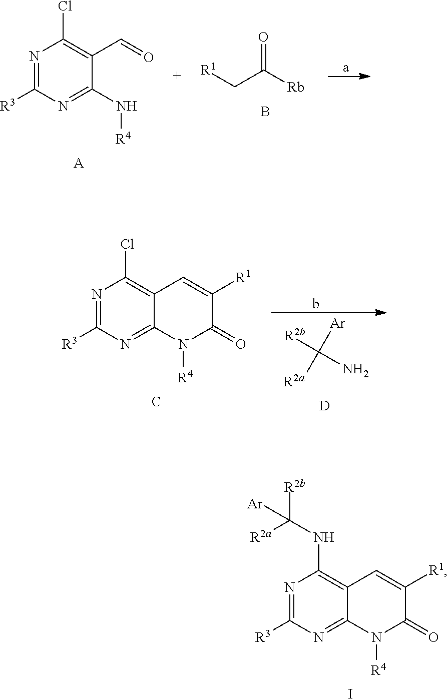

Step 1: 5-aldehyde-6-chloropyrimidine intermediate (1 eq.) and substituted acetate (3 eq.) are dissolved in an appropriate solvent and an inorganic base (3.5 eq.) is added at low temperature. The reaction solution is slowly warmed to room temperature and stirred overnight. The completion of the reaction is monitored by LC-MS. The reaction solution is added with water, the aqueous phase is extracted with ethyl acetate for three times, the extracted solution is dried over anhydrous sodium sulfate, concentrated under reduced pressure, the residue is separated and purified to obtain the target product, and the structure is confirmed by nuclear magnetic resonance and mass spectrometry.

Step 2: The intermediate of general formula (C) (1 eq.) which is the product of step 1 and compound D (1.2 eq.) are dissolved in a suitable solvent, and an organic base (2 eq.) is added. The reaction solution is heated to 100 degrees overnight. The completion of the reaction is monitored by TLC, and the residue is concentrated under reduced pressure. The residue is isolated and purified by silica gel column chromatography or HPLC preparation to obtain the target compound, and the structure is confirmed by nuclear magnetic resonance and mass spectrometry.

### Example General Preparation Method II

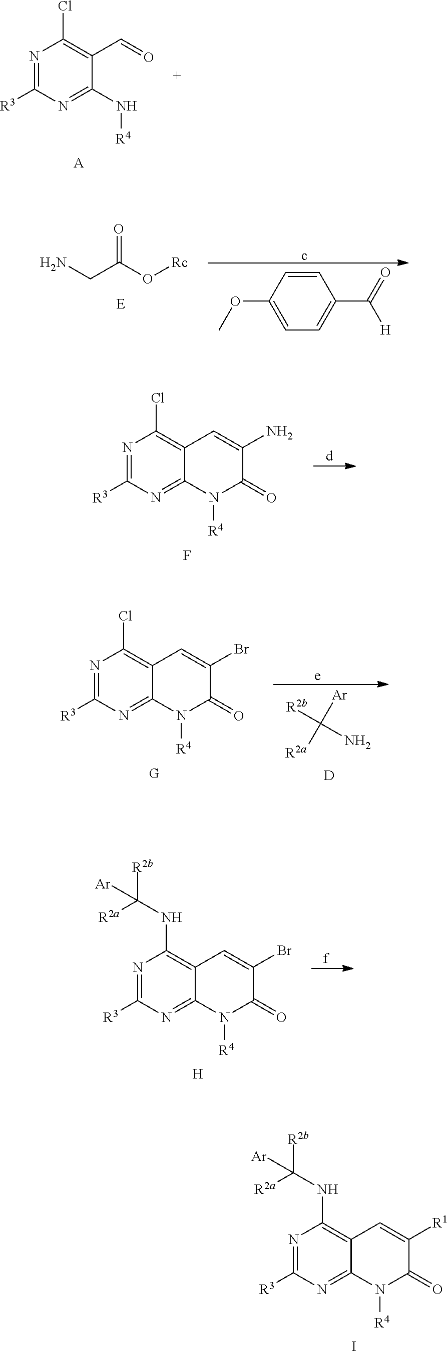

Step 1: 2-Aminoacetate (1.2 eq.) and p-methoxybenzaldehyde (1.2 eq.) are dissolved in a suitable solvent, a organic base (3 eq.) is added and stirred overnight at room temperature. 5-aldehyde-6-chloropyrimidine intermediate (1 eq.) is then added to the above reaction solution, and after stirring at room temperature overnight, acetic acid (30 eq.) is added. The reaction solution was heated to 60 degrees and stirred for 3-5 hours. After the basically completion of the reaction being monitored by LC-MS, the reaction solution is concentrated, and the crude product is purified by silica gel column chromatography to obtain the target product, and the structure is confirmed by nuclear magnetic resonance and mass spectrometry.

Step 2: After the above intermediate product of general formula (F)(1 eq.) being dissolved in an appropriate solvent, cuprous bromide (1.5 eq.) and tert-butyl nitrite (2 eq.) are added, the reaction solution is heated to 80 degrees and reacted for 2-3 hours. After the completion of the reaction being monitored by LC-MS, appropriate water is added, the mixture is extracted with ethyl acetate. The combined organic phases are concentrated and purified by silica gel column chromatography to obtain the target product, and the structure is confirmed by nuclear magnetic resonance and mass spectrometry.

Step 3: The intermediate of general formula (G) (1 eq.) which is the product of step 2 and compound D (1.2 eq.) are dissolved in a suitable solvent, and an organic base (2 eq.) is added. The reaction solution is heated to 100 degrees overnight. The completion of the reaction is monitored by TLC. The reaction is concentrated under reduced pressure. The residue is isolated and purified by silica gel column chromatography or HPLC preparation to obtain the target compound, and the structure is confirmed by nuclear magnetic resonance and mass spectrometry.

Step 4: the intermediate of general formula (H) (1 eq.) is dissolved in an appropriate solvent, the obtained solution and various borate or amino groups or alcohols (1-3 eq.) are heated to react for several hours under the catalytic action of transition metal complexes (0.1 eq.) and appropriate ligands (0.1 eq.). After the completion of the reaction being monitored by TLC or LC-MS, the reaction solution is filtered through celite, and the concentrated crude product is separated and purified by silica gel column chromatography or HPLC preparation to obtain the target compound of general formula (I), and the structure is confirmed by nuclear magnetic resonance and mass spectrometry.

### Example General Preparation Method III

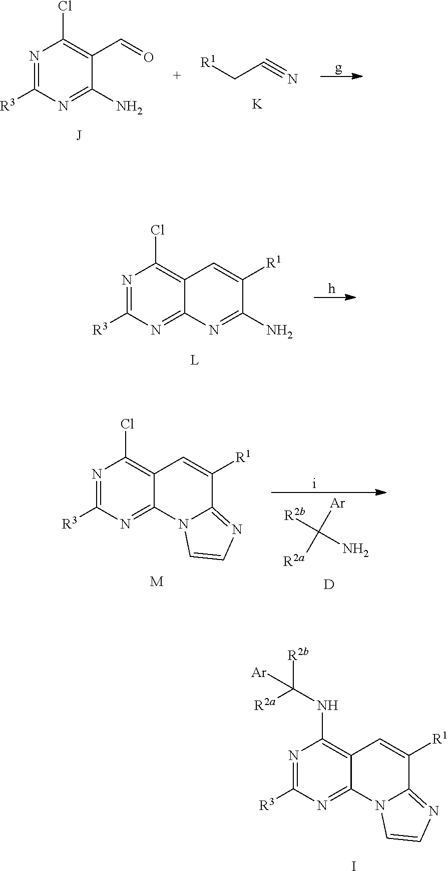

Step 1: Under the protection of nitrogen, the intermediate of general formula (J) (1 eq.) and the intermediate of general formula (K) (3 eq.) are dissolved in an appropriate solvent, and an organometallic base (3 eq.) is added at −78 degrees. After being stirred at this temperature for 10 minutes, the reaction is slowly warmed to room temperature and stirred overnight. LC-MS detection shows that the reaction does not proceed substantially, water is added to the reaction solution, extracted with ethyl acetate. After the organic phase is concentrated, the crude product is purified by silica gel column chromatography or HPLC to obtain the target product, and the structure is confirmed by nuclear magnetic resonance and mass spectrometry.

Step 2: The above intermediate of general formula (L) (1 eq.) and chloroacetaldehyde (1.2 eq.) are dissolved in a suitable solvent, an inorganic base (1.5 eq.) is added, and the reaction mixture is heated to 70-100 degrees and stirred overnight. The completion of the reaction is monitored by LC-MS, the reaction solution is concentrated, extracted three times with ethyl acetate after adding water. The combined organic phases are concentrated, and purified by silica gel column chromatography or HPLC preparation to obtain the target product, and the structure is confirmed by nuclear magnetic resonance and mass spectrometry.

Step 3: the above intermediate of general formula (M) (1 eq.) and the intermediate of general formula (D) (1.2 eq.) are dissolved in an appropriate solvent, and after adding an organic base (2 eq.), the reaction solution is heated to 100 degrees overnight. After the basically completion of the reaction being monitored by LC-MS, the reaction solution is concentrated under reduced pressure, and the crude product is purified by silica gel column chromatography or HPLC preparation to obtain the target product of general formula (I), and the structure is confirmed by nuclear magnetic resonance and mass spectrometry.

### Intermediate Preparation

### Intermediate 1: (R)-1-(2′-((dimethylamino) methyl)-[1,1′-biphenyl]-3-yl) ethyl-1-amine hydrochloride

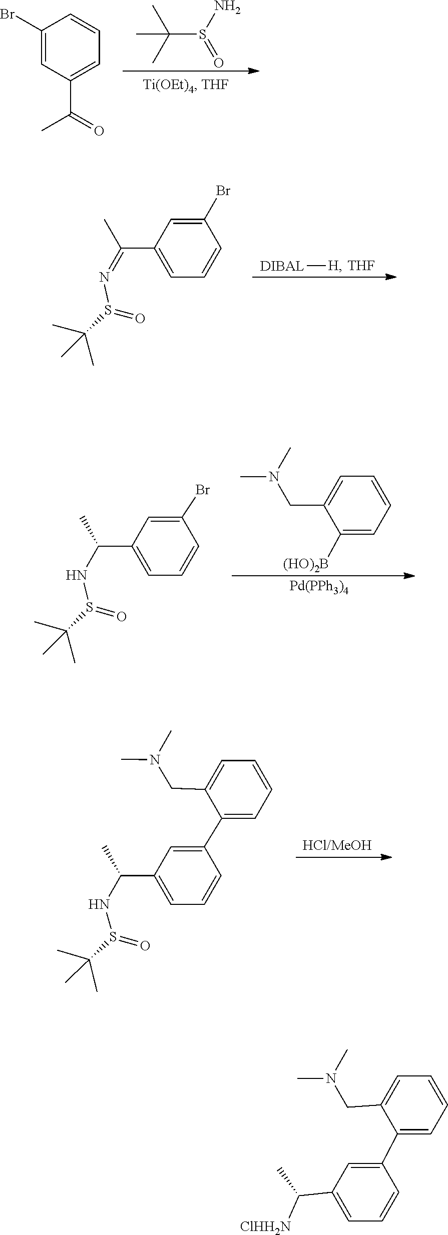

Step 1: Under nitrogen protection, tetraethyl titanate (11.3 g, 49.56 mmol) was added to 3-bromo-acetophenone (5.40 g, 27.26 mmol), (R)-(+) tert-butyl sulfinamide (3.0 g, 24.78 mmol) in tetrahydrofuran (42 mL). The reaction mixture was heated to 70° C. and reacted at this temperature for 16 hours. The reaction mixture was heated to 70° C. and reacted at this temperature for 16 hours. The reaction mixture was cooled to room temperature, 70 mL of brine was added, and the mixture was stirred for 10 minutes. The reaction mixture was filtered through celite and washed with ethyl acetate (100 mL) twice. The combined organic phases were dried over anhydrous sodium sulfate and filtered, and the filtrate was concentrated under reduced pressure. The crude product was purified by silica gel column chromatography (eluent: ethyl acetate/petroleum ether=1:4) to give a colorless oily intermediate compound (6.05 g). LCMS (ESI) m/z: 301.9 [M+H]⁺. ¹H NMR (400 MHz, DMSO-d6) δ 8.03 (s, 1H), 7.90 (d, J=7.8 Hz, 1H), 7.80-7.73 (m, 1H), 7.47 (m, 1H), 2.72 (s, 3H), 1.22 (s, 9H).

Step 2: At −78° C., diisobutylaluminum hydride (39.9 mL, 39.86 mmol) was added to the above intermediate (6.0 g, 19.93 mmol) in tetrahydrofuran (200 mL). The reaction solution was slowly warmed to room temperature and reacted at this temperature for 16 hours. Under ice-bath cooling, diluted sodium hydroxide solution was added to quench the reaction. The reaction mixture was filtered through celite and washed twice with ethyl acetate (100 mL). The combined organic phases were concentrated under reduced pressure, and the crude product was purified by silica gel column chromatography (eluent: petroleum ether/ethyl acetate=4:1) to give the compound (5.25 g) as a colorless oil. LCMS (ESI) m/z: 303.1 [M+H]⁺. ¹H NMR (400 MHz, DMSO-d6) δ 7.63 (s, 1H), 7.45-7.36 (m, 2H), 7.29 (m, 1H), 5.77 (d, J=7.7 Hz, 1H), 4.37 (m, 1H), 1.38 (d, J=6.8 Hz, 3H), 1.12 (s, 9H).

Step 3: Under nitrogen protection, tetrakis-triphenylphosphine palladium (1.52 g, 1.32 mmol) was added to a mixture of the above intermediate compound (4.0 g, 13.20 mmol), 2-(N,N-dimethylaminomethyl) benzeneboronic acid (3.07 g, 17.16 mmol), potassium carbonate (3.64 g, 26.40 mmol) and water (10 mL) in 1,4-dioxane (50 mL). The reaction mixture was heated to 100° C. and reacted at this temperature for 16 hours. The reaction mixture was diluted with ethyl acetate (200 mL) and washed with water (100 mL). The separated organic phase was concentrated under reduced pressure, and the crude product was purified by reverse phase column to give a brown oily compound (3.46 g). LCMS (ESI) m/z: 359.2 [M+H]⁺. ¹H NMR (400 MHz, DMSO-d6) δ 7.54-7.47 (m, 1H), 7.43 (s, 1H), 7.38 (d, J=4.8 Hz, 2H), 7.33 (m, 2H), 7.29-7.22 (m, 2H), 5.69 (m, 1H), 4.43 (m, 1H), 3.32 (s, 2H), 2.08 (s, 6H), 1.43 (d, J=6.8 Hz, 3H), 1.12 (s, 9H).

Step 4: A solution of hydrochloric acid in methanol (4M, 15 mL, 45 mmol) was added to the above intermediate compound (3.46 g, 9.66 mmol) in methanol (15 mL). The reaction mixture was reacted at 20 degrees for 2 hours. The reaction was detected to be substantially completed by LC-MS, and after the solvent was removed by concentration under reduced pressure, the residue was purified by silica gel column chromatography (dichloromethane/methanol=9:1) to give a pale yellow solid compound (2.5 g). LCMS (ESI) m/z: 255.2 [M+H]⁺. ¹H NMR (400 MHz, CD₃OD) δ 7.82-7.69 (m, 1H), 7.66-7.53 (m, 4H), 7.50 (s, 1H), 7.41 (d, J=6.1 Hz, 2H), 4.59 (m, 1H), 4.42 (s, 2H), 2.67 (s, 6H), 1.70 (d, J=6.2 Hz, 3H).

Preparation conditions: separation column (SunFire Prep C18 OBD™, 10 um, 19*250 mm); Gradient (5%-95% acetonitrile/0.1 formic acid/water, 16 min, flow rate 20 mL/min).

Analysis conditions: analysis column (Waters SunFire C18, 4.6*50 mm, 5 um); Gradient (5%-95% acetonitrile/0.1 formic acid/water, 3.0 min, flow rate 2.0 mL/min, 2.6 min); Column temperature: 40° C.; Detection wavelength: 254 nM.

### Intermediate 2: (R)-1-(5-(2-(Pyrrol-1-ylmethyl) phenyl) thiophen-2-yl) ethyl-1-amine hydrochloride

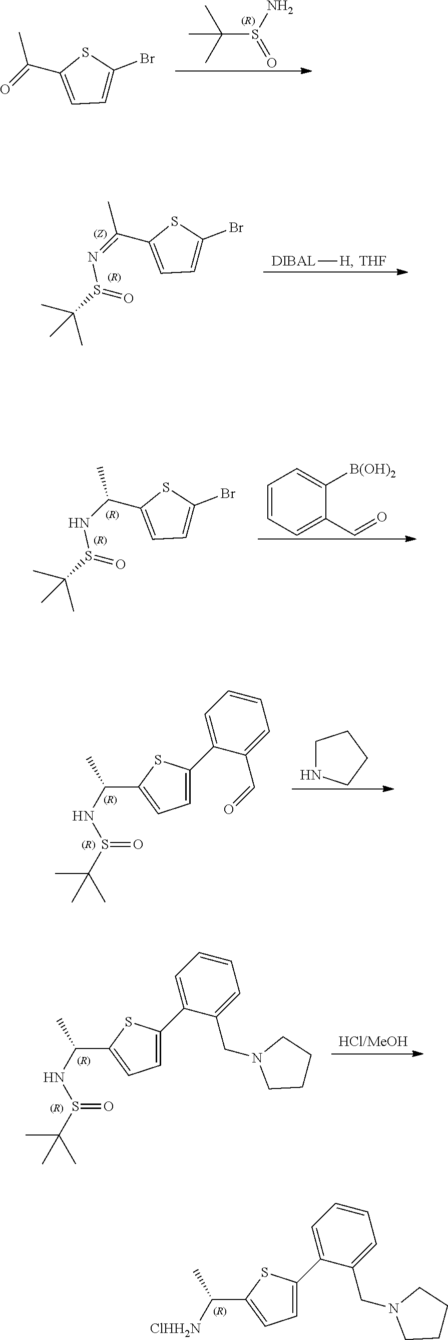

Step 1: Under nitrogen protection, tetraethyl titanate (30.1 g, 132 mmol) was added to 1-(5-bromothiophen-2-yl) ethyl-1-one (14.9 g, 72.61 mmol), (R)-(+) tert-butyl sulfinamide (8.0 g, 66 mmol) in tetrahydrofuran (100 mL), the reaction mixture was heated to 70° C. and reacted at this temperature for 16 hours. After the reaction solution was cooled to room temperature, 100 mL of brine was added, and the mixture was continued to stir for 10 minutes. The reaction mixture was filtered through celite, and the filtrate was extracted twice with ethyl acetate (100 mL). The combined organic phases were dried over anhydrous sodium sulfate, concentrated, and the crude product was purified by silica gel column chromatography (eluent: ethyl acetate/petroleum ether=4:1) to give the intermediate compound as a brown solid (15 g, crude product). LCMS (ESI) m/z: 307.9 [M+H]⁺. ¹H NMR (400 MHz, DMSO-d6) δ 7.64 (d, J=4.1 Hz, 1H), 7.35 (d, J=4.1 Hz, 1H), 2.64 (s, 3H), 1.18 (s, 9H).

Step 2: Under nitrogen protection, under cooling at −78° C., diisobutylaluminum hydride (DIBAL-H)(61 mL, 61 mmol) was slowly added dropwise to the above intermediate compound (9.3 g, 30.17 mmol) in tetrahydrofuran (200 mL), and the reaction mixture was slowly warmed to room temperature and reacted at this temperature for 16 hours. LC-MS detection showed that there was no starting material, most of it was converted into the desired product. Methanol (50 mL) was added for quenching, and the solvent was removed by concentration under reduced pressure. The crude product was slurried with methanol (200 mL) and filtered through celite. The filtrate was concentrated under reduced pressure and purified by silica gel column chromatography (eluent: ethyl acetate/petroleum ether=4:1) to give a brown oily liquid compound (15 g, crude product). LCMS (ESI) m/z: 309.9 [M+H]⁺. ¹H NMR (400 MHz, DMSO-d6) δ 7.06 (d, J=3.8 Hz, 1H), 6.89 (dd, J=3.8, 0.9 Hz, 1H), 5.90 (d, J=7.1 Hz, 1H), 4.57 (m, 1H), 1.47 (d, J=6.8 Hz, 3H), 1.12 (s, 9H).

Step 3: Under nitrogen protection, tetrakistriphenylphosphine palladium (1.12 g, 0.965 mmol) was added to a mixture of the above intermediate compound (3 g, 9.65 mmol), 2-carboxaldehyde benzeneboronic acid (1.88 g, 12.55 mmol), potassium carbonate (2.67 g, 19.3 mmol), water (12 mL) in 1,4-dioxane (60 mL). The reaction mixture was heated to 100° C. and reacted at this temperature for 16 hours. The reaction solution was diluted with ethyl acetate (200 mL) and then washed with water (100 mL). The separated organic phase was dried over anhydrous sodium sulfate and concentrated under reduced pressure. The crude product was purified by HPLC preparation to give the intermediate compound (2.6 g) as a brown oil. LCMS (ESI) m/z: 336.0 [M+H]⁺. ¹H NMR (400 MHz, CDCl3) δ 10.21 (d, J=0.6 Hz, 1H), 8.00 (dd, J=7.8, 1.1 Hz, 1H), 7.61 (m, 1H), 7.55-7.46 (m, 2H), 7.08 (d, J=3.0 Hz, 1H), 6.92 (d, J=3.6 Hz, 1H), 4.90-4.82 (m, 1H), 3.58 (d, J=3.6 Hz, 1H), 1.66 (d, J=6.6 Hz, 3H), 1.26 (s, 9H).

Step 4: At room temperature, 1 drop of glacial acetic acid was added to a solution of the above intermediate compound (2.6 g, 7.75 mmol) and tetrahydropyrrole (662 mg, 9.3 mmol) in methanol (30 mL), and the reaction mixture was reacted at 20° C. for 2 hours. Then, sodium cyanoborohydride (1.46 g, 23.25 mmol) was added into the above reaction solution, and the reaction was continued for 12 hours. LCMS detection showed that the product was main. The crude product obtained by concentrating under reduced pressure to remove the solvent was purified by silica gel column chromatography (eluent: dichloromethane/methanol=9:1) to give the intermediate compound (2.1 g) as a pale yellow solid. LCMS (ESI) m/z: 391.1 [M+H]⁺.

Step 5: 3N hydrogen chloride/methanol solution HCl(g)/MeOH (15 mL, 45 mmol) was added to the above intermediate compound (2.1 g, 5.38 mmol) in methanol (15 mL). The reaction mixture was reacted at 20 degrees for 2 hours. LC-MS detection showed that the reaction was completed. After the reaction solution was concentrated under reduced pressure to remove the solvent, the resulting crude product was purified by silica gel column chromatography (eluent: dichloromethane/methanol=9:1) to give the target compound as a pale yellow solid (1.2 g). LCMS (ESI) m/z: 287.1 [M+H]⁺. ¹H NMR (400 MHz, CD₃OD) δ 7.79-7.75 (m, 1H), 7.60-7.50 (m, 3H), 7.32 (d, J=3.6 Hz, 1H), 7.13 (d, J=3.6 Hz, 1H), 4.83 (m, 1H), 4.58 (s, 2H), 3.54-3.44 (m, 2H), 3.02 (d, J=8.1 Hz, 2H), 2.06-1.94 (m, 4H), 1.77 (d, J=6.9 Hz, 3H).

### Intermediate 3: (R)-1-(5-(2-((dimethylamino) methyl) phenyl) thiophen-2-yl) ethyl-1-amine hydrochloride

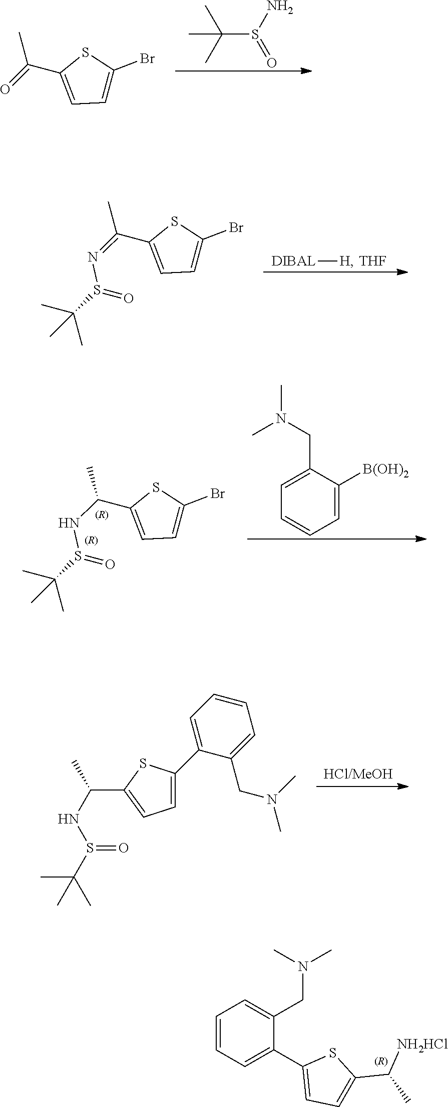

Step 1: Tetraethyl titanate (17.3 mL, 82.92 mmol) was added to a solution of 2-acetyl-5-bromo-thiophene (9.3 g, 45.61 mmol) and (R)-(+) tert-butylsulfinamide (5.0 g, 41.46 mmol) in tetrahydrofuran (70 mL) under nitrogen protection. The reaction mixture was heated to 70° C. and reacted at this temperature for 16 hours. The reaction mixture was cooled to room temperature, 70 mL of brine was added, and the mixture was continued to stir for 10 minutes. The reaction mixture was filtered through celite and washed with ethyl acetate (100 mL) twice. The combined organic phases were dried over anhydrous sodium sulfate and filtered, and the filtrate was concentrated under reduced pressure. The crude product was purified by silica gel column chromatography (ethyl acetate/petroleum ether=1:4) to give a colorless oily intermediate compound (8.56 g). LCMS (ESI) m/z: 309.9 [M+H]^{+.1}H NMR (400 MHz, DMSO-d6) δ 7.64 (d, J=4.1 Hz, 1H), 7.35 (d, J=4.1 Hz, 1H), 2.64 (s, 3H), 1.18 (s, 9H).

Step 2: At −78° C., diisobutylaluminum hydride (55 mL, 55.4 mmol) was added to the above intermediate (8.5 g, 27.69 mmol) in tetrahydrofuran (200 mL). The reaction solution was slowly warmed to room temperature and reacted at this temperature for 16 hours. Under ice-bath cooling, dilute sodium hydroxide solution was added to quench the reaction. The reaction mixture was filtered through celite and washed twice with ethyl acetate (100 mL). The combined organic phases were concentrated under reduced pressure, and the crude product was purified by silica gel column chromatography (eluent: petroleum ether/ethyl acetate=4:1) to give the compound (7.97 g) as a colorless oil. LCMS (ESI) m/z: 312.0 [M+H]⁺. ¹H NMR (400 MHz, DMSO-d6) δ 7.06 (d, J=3.8 Hz, 1H), 6.89 (dd, J=3.8, 0.9 Hz, 1H), 5.90 (d, J=7.1 Hz, 1H), 4.57 (m, 1H), 1.47 (d, J=6.8 Hz, 3H), 1.12 (s, 9H).

Step 3: Under nitrogen protection, tetrakistriphenylphosphine palladium (2.48 g, 2.146 mmol) was added to a solution of the above intermediate compound (6.63 g, 21.46 mmol), 2-(N,N-dimethylaminomethyl) benzeneboronic acid (5.0 g, 27.92 mmol), potassium carbonate (5.92 g, 42.91 mmol) and water (10 mL) in 1,4-dioxane (50 mL). The reaction mixture was heated to 100° C. and reacted at this temperature for 16 hours. The reaction mixture was diluted with ethyl acetate (200 mL) and then washed with water (100 mL). The separated organic phase was concentrated under reduced pressure, and the crude product was purified by reverse phase column to give a brown oily compound (6.0 g). LCMS (ESI) m/z: 365.1 [M+H]⁺. ¹H NMR (400 MHz, DMSO-d6) δ 7.47-7.43 (m, 1H), 7.41-7.37 (m, 1H), 7.32 (m, 2H), 7.16 (d, J=3.6 Hz, 1H), 7.07 (dd, J=3.6, 0.9 Hz, 1H), 5.88 (d, J=7.1 Hz, 1H), 4.65 (m, 1H), 3.39 (s, 2H), 2.14 (s, 6H), 1.55 (d, J=6.8 Hz, 3H), 1.14 (s, 9H).

Step 4: A solution of hydrochloric acid in methanol (4M, 15 mL, 45 mmol) was added to the above intermediate compound (2.5 g, 6.865 mmol) in methanol (15 mL). The reaction mixture was reacted at 20 degrees for 2 hours. The reaction was detected to be substantially completed by LC-MS, and after the solvent being removed by concentration under reduced pressure, the residue was purified by silica gel column chromatography (dichloromethane/methanol=9:1) to give a pale yellow solid compound (1.6 g). LCMS (ESI) m/z: 261.1 [M+H]⁺. ¹H NMR (400 MHz, CD₃OD) δ 7.77-7.71 (m, 1H), 7.63-7.49 (m, 3H), 7.32 (d, J=3.6 Hz, 1H), 7.13 (d, J=3.6 Hz, 1H), 4.83 (m, 1H), 4.53 (s, 2H), 2.76 (s, 6H), 1.78 (d, J=6.9 Hz, 3H).

Preparation conditions: separation column (SunFire Prep C18 OBD™, 10 um, 19*250 mm); Gradient (5%-95% acetonitrile/0.1 formic acid/water, 16 min, flow rate 20 mL/min).

Analysis conditions: analysis column (Waters SunFire C18, 4.6*50 mm, 5 um); Gradient (5%-95% acetonitrile/0.1 formic acid/water, 3.0 min, flow rate 2.0 mL/min, 2.6 min); Column temperature: 40° C.; Detection wavelength: 254 nM.

Referring to the route and operation method for the preparation of intermediates 1, 2 and 3, the following intermediates 4-16 were prepared:

| No. | structure | analysis data |
|---|---|---|
| 4 | 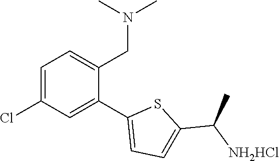 | LCMS (ESI) m/z: 295.1 [M + H]⁺. ¹H NMR (400 MHz, CD₃OD) δ 7.93-7.87 (m, 1H), 7.53-7.45 (m, 2H), 7.32 (d, J = 3.6 Hz, 1H), 7.13 (d, J = 3.6 Hz, 1H), 4.85 (m, 1H), 4.55 (s, 2H), 2.78 (s, 6H), 1.78 (d, J = 6.9 Hz, 3H). |
| 5 | 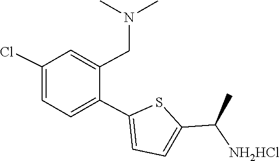 | LCMS (ESI) m/z: 294.9 [M + H]+. ¹H NMR (400 MHz, CD₃OD) δ 7.73-7.67 (m, 1H), 7.55-7.48 (m, 2H), 7.23 (d, J = 3.6 Hz, 1H), 7.07 (d, J = 3.6 Hz, 1H), 4.85 (m, 1H), 4.55 (s, 2H), 2.78 (s, 6H), 1.73 (d, J = 6.9 Hz, 3H). |
| 6 | 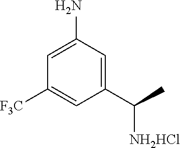 | LCMS (ESI) m/z: 205.2 [M + H]⁺ ¹H NMR (400 MHz, CD₃OD): δ 7.10 (s, 1H), 7.05 (s, 1H), 6.92 (s, 1H), 5.55-5.51 (m, 1H), 1.63 (d, J = 6.8 Hz, 3H). |
| 7 | 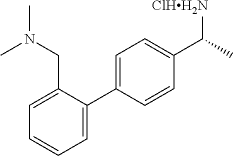 | LCMS (ESI) m/z: 255.2 [M + H]⁺. ¹H NMR (400 MHz, MeOD-d₄): δ 7.51-7.63 (m, 6H), 7.37-7.39 (m, 1H), 7.31 (d, J = 8.0 Hz, 1H), 4.63- 4.55 (m, 1H), 4.38 (s, 2H), 2.61 (s, 6H), 1.70 (d, J = 7.2 Hz, 3H). |
| 8 | 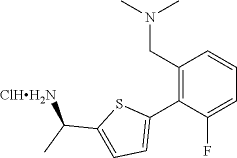 | LCMS (ESI) m/z: 279.1 [M + H]⁺. ¹H NMR (400 MHz, MeOD) δ 7.77- 7.53 (m, 3H), 7.28 (d, J = 3.6 Hz, 1H), 7.15 (d, J = 3.6 Hz, 1H), 4.83 (m, 1H), 4.52 (s, 2H), 2.76 (s, 6H), 1.76 (d, J = 6.9 Hz, 3H). |
| 9 | 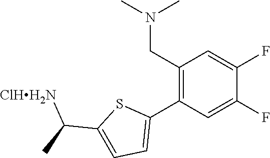 | LCMS (ESI) m/z: 297.1 [M + H]⁺. ¹H NMR (400 MHz, MeOD) δ 7.65- 7.43 (m, 2H), 7.32 (d, J = 3.6 Hz, 1H), 7.13 (d, J = 3.6 Hz, 1H), 4.83 (m, 1H), 4.55 (s, 2H), 2.73 (s, 6H), 1.79 (d, J = 6.9 Hz, 3H). |
| 10 | 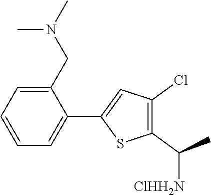 | LCMS (ESI) m/z: 295.1/297.1 [M + H]⁺. ¹H NMR (400 MHz, MeOD) δ 7.77-7.52 (m, 4H), 7.16 (s, 1H), 4.93 (m, 1H), 4.52 (s, 2H), 2.76 (s, 6H), 1.75 (d, J = 6.9 Hz, 3H). |
| 11 | 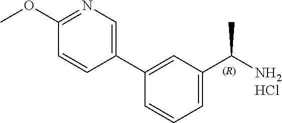 | LCMS (ESI) m/z: 229.1 [M + H]⁺. ¹H NMR (400 MHz, DMSO-d₆) δ 8.53 (m, 1H), 8.46 (m, 1H), 8.00 (m, 1H), 7.54 (m, 1H), 7.44 (m, 2H), 6.91 (m, 1H), 5.61-5.64 (m, 1H), 3.89 (s, 3H), 1.69 (d, J = 7.2 Hz, 3H). |
| 12 | 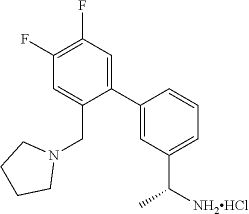 | LCMS (ESI) m/z: 317.2 [M + H]⁺. ¹H NMR (400 MHz, CD₃OD-d₄) δ 7.82-7.53 (m, 4H), 7.50 (s, 1H), 7.41 (m, 1H), 4.59 (m, 1H), 4.42 (s, 2H), 2.41-2.44 (m, 4H), 1.77-1.74 (m, 4H), 1.70 (d, J = 6.2 Hz, 3H). |
| 13 | 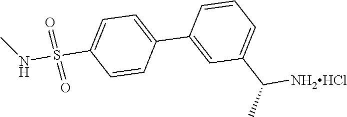 | LCMS (ESI) m/z: 291.1 [M + H]+. ¹HNMR (400 MHz, CD₃OD-d₄): δ 7.94 (m, 2H), 7.71-7.74 (m, 2H), 7.43-7.55 (m, 4H), 5.65-5.70 (m, 1H), 3.79-3.73 (m, 3H), 1.72 (d, J = 6.8 Hz, 3H). |
| 14 | 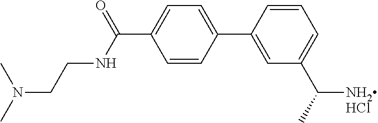 | LCMS (ESI) m/z: 312.2 [M + H]⁺. ¹HNMR (400 MHz, CD₃OD-d₄): δ 7.94 (d, J = 8.4 Hz, 2H), 7.71-7.74 (m, 2H), 7.43-7.55 (m, 4H), 5.65- 5.70 (m, 1H), 3.76-3.79 (m, 2H), 3.38-3.41 (m, 2H), 2.99 (s, 6H), 1.70 (d, J = 6.8 Hz, 3H). |
| 15 | 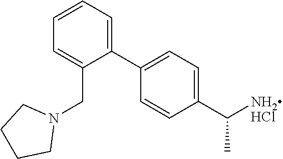 | LCMS (ESI) m/z: 281.2 [M + H]⁺. ¹H NMR (400 MHz, MeOD-d₄): δ 7.60-7.59 (m, 1H), 7.51-7.41 (m, 4H), 7.35-7.31 (m, 3H), 5.45-5.42 (m, 1H), 3.67 (s, 2H), 2.41-2.44 (m, 4H), 1.77-1.74 (m, 4H), 1.63- 1.61 (d, J = 7.2 Hz, 3H). |
| 16 | 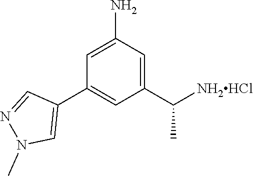 | LCMS (ESI) m/z: 217.2 [M + H]⁺. ¹H NMR (400 MHz, CD₃OD): δ 8.85 (s, 1H), 8.20 (s, 1H), 7.59 (s, 1H), 7.18 (s, 1H), 7.08 (s, 1H), 5.57-5.51 (m, 1H), 3.93 (s, 3H), 1.63 (d, J = 6.8 Hz, 3H). |

### Example 1: (R)-2,8-dimethyl-6-(tetrahydropyran-4-yl)-4-((1-(3-(trifluoromethoxy) phenyl) ethyl) amino) pyrido (2,3-d) pyrimidin-7 (8H)-one

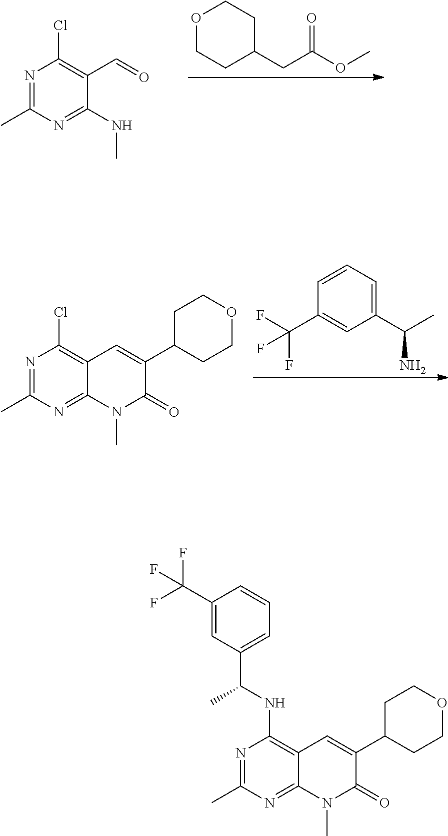

Step 1: 4-chloro-2-methyl-6-(methylamino) pyrimidine-5-carbaldehyde (50 mg, 0.3 mmol) and methyl 2-(tetrahydropyran-4-yl)-acetate (142 mg, 0.9 mmol) were dissolved in tetrahydrofuran (THF)(20 mL), after cooling to −78 degrees, hexamethylaminosilyl lithium salt (LIHMDS) (1 mL, 1.0 mmol) was added dropwise. The reaction solution was slowly warmed to room temperature, then reacted overnight. LC-MS detection showed that the reaction was completed, water (40 mL) and ethyl acetate (80 mL) were added to the reaction mixture. The separated aqueous phase was extracted twice with ethyl acetate (40 mL), and the organic phases were combined and concentrated under reduced pressure. The crude product was purified by column chromatography (eluent: petroleum ether/ethyl acetate=volume ratio of 5:1) to obtain white solid intermediate product (28 mg). LC-MS [M+H]⁺: m/z 294.1.

Step 2: the above intermediate product (28.0 mg, 0.09 mmol) and (R)-1-(3-(trifluoromethyl) phenyl) ethyl-1-amine (22.7 mg, 0.12 mmol) were dissolved in acetonitrile (10 mL), N,N-diisopropylethylamine (DIEA) solution (18.2 mg, 0.18 mmol) was added, the reaction solution was heated to 100 degrees and reacted overnight. After LC-MS detection showed that the reaction was completed, the reaction solution was concentrated under reduced pressure, and the crude product was separated to obtain a light yellow solid product (33.1 mg) by HPLC preparation. LC-MS [M+H]: m/z 447.2. ¹M H-NMR (400 MHz, DMSO-d6): 8.21 (d, J=7.6 Hz, 1H), 8.12 (s, 1H), 7.78 (s, 1H), 7.73 (d, J=6.4 Hz, 1H), 7.57-7.59 (m, 2H), 5.59-5.61 (i, 1H), 3.96-3.99 (m, 2H), 3.56 (s, 3H), 3.42-3.58 (i, 2H), 3.01-3.10 (m, 1H), 2.36 (s, 3H), 1.68-1.73 (N, 4H), 1.59 (d, J 7.2 Hz, 3H).

Referring to the method of Example 1, Examples 2-7 were obtained by the synthesis method wherein methyl 2-(tetrahydropyran-4-yl)-acetate was replaced with different 2-substituted acetate as raw material and (R)-1-(3-(trifluoromethyl) phenyl) ethyl-1-amine was replaced with No structure analysis data (LC-MS and ¹H-NMR)

| No | structure | analysis data (LC-MS and ¹H-NMR) |
|---|---|---|
| 2 | 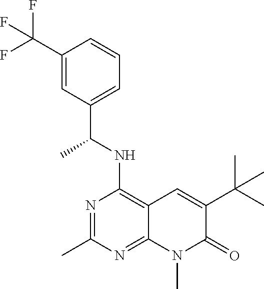 [SMILES: Cc1nc(N[C@H](C)c2cccc(C(F)(F)F)c2)c2cc(C(C)(C)C)c(=O)n(C)c2n1] | (ESI) [M + H]⁺: m/z 419.1. (400 MHz, DMSO-d₆): δ 8.29 (d, J = 8.0 Hz, 1H), 8.01 (s, 1H), 7.79 (s, 1H), 7.74 (d, J = 6.8 Hz, 1H), 7.57-7.59 (m, 2H), 5.59-5.64 (m, 1H), 3.55 (s, 3H), 2.36 (s, 3H), 1.60 (d, J = 7.2 Hz, 3H), 1.38 (s, 9H). |
| 3 | 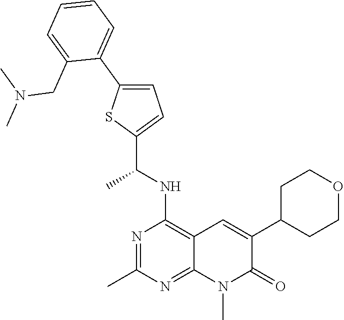 [SMILES: Cc1nc(N[C@H](C)c2ccc(-c3ccccc3CN(C)C)s2)c2cc(C3CCOCC3)c(=O)n(C)c2n1] | (ESI) [M + H]⁺: m/z 518.2. (400 MHz, DMSO-d₆): δ 8.33 (d, J = 8.4 Hz, 1H), 8.09 (s, 1H), 7.39-7.43 (m, 1H), 7.37-7.38 (m, 1H), 7.34-7.37 (m, 2H), 7.18 (d, J = 4.0 Hz, 1H), 7.07 (d, J = 4.0 Hz, 1H), 5.89-5.93 (m, 1H), 3.93-3.97 (m, 2H), 3.58 (s, 3H), 3.41-3.50 (m, 2H), 3.29 (s, 2H), 3.01-3.05 (m, 1H), 2.44 (s, 3H), 2.10 (s, 6H), 1.60-1.70 (m, 7H). |
| 4 | 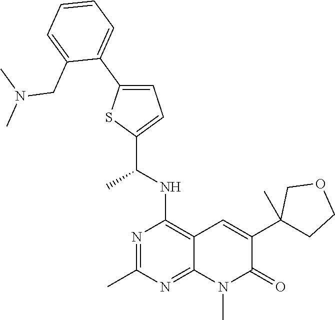 [SMILES: Cc1nc(N[C@H](C)c2ccc(-c3ccccc3CN(C)C)s2)c2cc(C3(C)CCOC3)c(=O)n(C)c2n1] | (ESI) m/z: 518.3 [M + H]⁺. (400 MHz, DMSO-d₆): δ 8.33 (m, 1H), 8.09 (s, 1H), 7.43-7.35 (m, 4H), 7.18 (m, 1H), 7.07 (d, J = 4.0 Hz, 1H), 5.89-5.93 (m, 1H), 3.97-3.77 (m, 5H), 3.41-3.50 (m, 2H), 3.29 (s, 2H), 3.01-3.05 (m, 1H), 2.44 (s, 3H), 2.10 (s, 6H), 1.70-1.53 (m, 7H). |
| 5 | 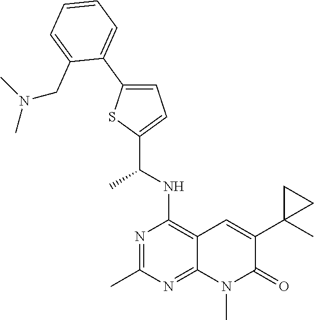 [SMILES: Cc1nc(N[C@H](C)c2ccc(-c3ccccc3CN(C)C)s2)c2cc(C3(C)CC3)c(=O)n(C)c2n1] | (ESI) m/z: 488.2 [M + H]⁺. (400 MHz, DMSO-d₆): δ 8.35 (m, 1H), 8.12 (s, 1H), 7.43-7.37 (m, 4H), 7.18 (m, 1H), 7.07 (d, J = 4.0 Hz, 1H), 5.89-5.93 (m, 1H), 3.41-3.50 (m, 2H), 3.29 (s, 2H), 2.44 (s, 3H), 2.10 (s, 6H), 1.70-1.53 (s, 3H), 0.7 (m, 2H), 0.5 (m, 2H). |
| 6 | 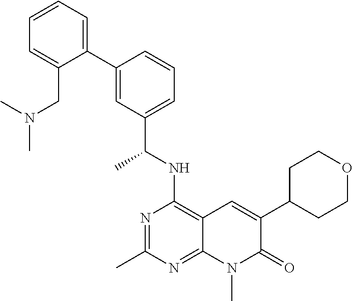 [SMILES: Cc1nc(N[C@H](C)c2cccc(-c3ccccc3CN(C)C)c2)c2cc(C3CCOCC3)c(=O)n(C)c2n1] | (ESI) m/z: 512.3 [M + H]⁺. (400 MHz, DMSO-d₆): δ 7.63 (d, J = 7.9 Hz, 1H), 7.49-7.43 (m, 3H), 7.38-7.36 (m, 3H), 7.32-7.27 (m, 2H), 7.21 (m, 1H), 5.93 (m, 1H), 4.42 (s, 2H), 4.02-3.96 (m, 2H), 3.58 (s, 3H), 3.59-3.41 (m, 2H), 3.12-3.02 (m, 1H), 2.38 (s, 3H), 1.75-1.68 (m, 4H), 1.59 (d, J = 7.2 Hz, 3H). |
| 7 | 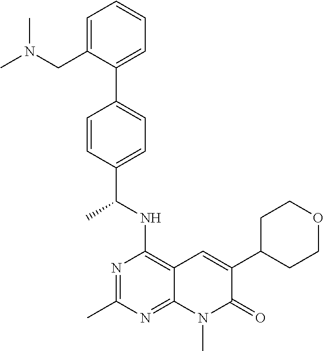 [SMILES: Cc1nc(N[C@H](C)c2ccc(-c3ccccc3CN(C)C)cc2)c2cc(C3CCOCC3)c(=O)n(C)c2n1] | (ESI) m/z: 512.3 [M + H]⁺. (400 MHz, DMSO-d₆): δ 8.54 (d, J = 7.9 Hz, 1H), 7.49 (m, 3H), 7.41-7.34 (m, 3H), 7.34-7.27 (m, 2H), 7.21 (m, 1H), 5.70 (m, 1H), 4.10 (s, 2H), 3.99-3.96 (m, 2H), 3.56 (s, 3H), 3.58- 3.42 (m, 2H), 3.10-3.01 (m, 1H), 2.36 (s, 3H), 1.73-1.68 (m, 4H), 1.59 (d, J = 7.2 Hz, 3H). |

### Example 8: (R)-2,8-dimethyl-6-(1-methyl-6-oxo-1,6-dihydropyridin-3-yl)-4-((1-(3-(trifluoromethoxy) phenyl) ethyl) amino) pyrido (2,3-d) pyrimidin-7 (8H)-one

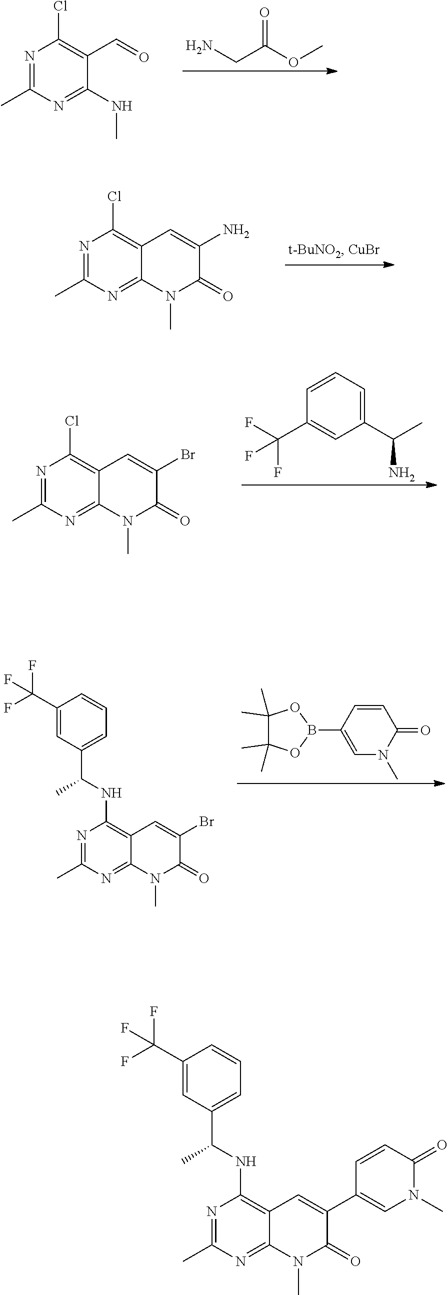

Step 1: methyl 2-aminoacetate (152 mg, 1.2 mmol) and p-methoxybenzaldehyde (164 mg, 1.2 mmol) were dissolved in methanol (30 mL), triethylamine (334 mg, 3.3 mmol) was added. After the reaction being stirred overnight at room temperature, 4-chloro-2-methyl-6-(methylamino) pyrimidine-5-carbaldehyde (200 mg, 1.0 mmol) was further added, and the reaction was continued to stir at room temperature overnight. After acetic acid (3 mL) being added to the reaction solution, the mixture was heated to 65 degrees and stirred for 3 hours. The reaction solution was concentrated under reduced pressure, and the crude product was purified by silica gel column chromatography (eluent: petroleum ether/ethyl acetate=5/1) to give a white solid product (80.1 mg). LC-MS[M+H]⁺: m/z 225.2.

Step 2: The above intermediate product (80 mg, 0.36 mmol) was dissolved in acetonitrile (30 mL), and cuprous bromide (CuBr) (80 mg, 0.56 mmol) and tert-butyl nitrite (72.0 mg, 0.72 mmol) were added. After being heated to 80 degrees, the reaction was conducted for 5 hours. Water (50 mL) and ethyl acetate (50 mL) were added to the reaction mixture, and the mixture was extracted twice with ethyl acetate (50 mL). The combined organic phases were concentrated under reduced pressure, and the crude product was purified and separated by silica gel column chromatography (eluent: petroleum ether/ethyl acetate=5/1) to give a white solid product (20 mg). LC-MS[M+H]⁺: m/z 288.0.

Step 3: the above intermediate compound (20 mg, 0.06 mmol) and (R)-1-(3-(trifluoromethyl) phenyl) ethyl-1-amine (14 mg, 0.06 mmol) were dissolved in acetonitrile (10 mL), DIEA (18.2 mg, 0.18 mmol) was added, and the mixture was heated to 100 degrees to react overnight. The reaction solution was concentrated under reduced pressure and dried to give a yellow crude compound (10 mg). LC-MS[M+H]⁺: m/z 441.1.

Step 4: under nitrogen protection, the above intermediate compound (10.1 mg, 0.02 mmol) and 1-methyl-5-(4,4,5,5-tetramethyl-1,3,2-dioxaborinane-2-yl) pyridin-2 (1H)-one (5.1 mg, 0.02 mmol), [1,1′-Bis (diphenylphosphino) ferrocene] palladium dichloride Pd(dppf)Cl₂ (1.0 mg, 0.002 mmol) and potassium phosphate (14.2 mg, 0.06 mmol) were dissolved in dioxane/water (10 mL/2 mL), and the mixture was heated to 90 degrees to react overnight. The reaction solution was concentrated under reduced pressure, and the crude product was separated to obtain a white solid product (5.5 mg) by HPLC preparation. LC-MS[M+H]⁺: m/z 470.1. ¹H NMR (400 MHz, DMSO-d6): δ 8.45 (s, 1H), 8.38 (d, J=2.4 Hz, 1H), 8.32 (d, J=7.6 Hz, 1H), 7.85 (dd, J=9.6, 2.4 Hz, 1H), 7.79 (s, 1H), 7.74 (d, J=6.8 Hz, 1H), 7.57-7.61 (m, 2H), 6.52 (d, J=9.6 Hz, 1H), 5.60-5.65 (m, 1H), 3.61 (s, 3H), 3.52 (s, 3H), 2.38 (s, 3H), 1.60 (d, J=7.2 Hz, 3H).

Referring to the method of Example 8, Examples 9-11 were obtained by the synthesis method wherein 1-methyl-5-(4,4,5,5-tetramethyl-1,3,2-dioxaboran-2-yl) pyridin-2(1H)-one was replaced with different boric acid or boric acid ester, tin reagent as raw material and (R)-1-(3-(trifluoromethyl)phenyl) ethyl-1-amine was replaced with different amine reagents as raw materials;

| No | structure | analysis data (LC-MS and ¹H-NMR) |
|---|---|---|
| 9 | 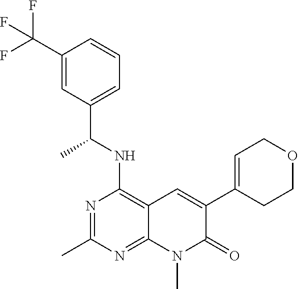 [SMILES: Cc1nc(N[C@H](C)c2cccc(C(F)(F)F)c2)c2cc(C3=CCOCC3)c(=O)n(C)c2n1] | (ESI) [M + H]⁺: m/z 445.1. (400 MHz, DMSO-d₆): δ 8.33 (d, J = 7.2 Hz, 1H), 8.19 (s, 1H), 7.79 (s, 1H), 7.73 (d, J = 7.2 Hz, 1H), 7.56- 7.61 (m, 2H), 6.63 (s, 1H), 5.60-5.63 (m, 1H), 4.24 (ds, J = 2.4 Hz, 2H), 3.82 (t, J = 5.2 Hz, 2H), 3.56 (s, 3H), 2.49-2.51 (m, 2H), 2.37 (s, 3H), 1.59 (d, J = 6.8 Hz, 3H). |
| 10 | 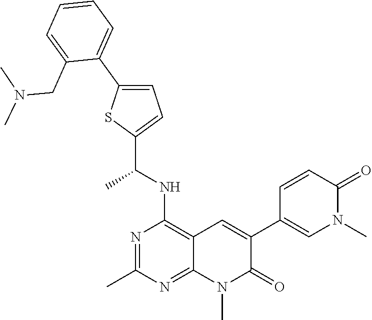 | (ESI) m/z: 541.2 [M + H]⁺. (400 MHz, DMSO-d₆): δ 8.82 (m, 1H), 8.45 (s, 1H), 8.38 (m, 1H), 8.32 (d, J = 7.6 Hz, 1H), 7.85 (m, 1H), 7.46-7.42 (m, 1H), 7.33-7.30 (m, 2H), 7.20 (d, J = 3.6 Hz, 1H), 7.11 (d, J = 3.6 Hz, 1H), 6.52 (d, J = 8.4 Hz, 1H), 6.05 (s, 1H), 5.60-5.65 (m, 1H), 3.61 (s, 3H), 3.52 (s, 3H), 3.37 (s, 2H), 2.38 (s, 3H), 1.60 (d, J = 7.2 Hz, 3H), 2.12 (s, 6H). |
| 11 | 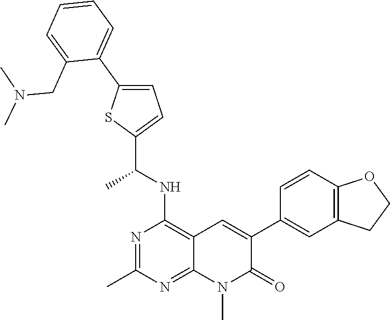 [SMILES: Cc1nc(N[C@H](C)c2ccc(-c3ccccc3CN(C)C)s2)c2cc(-c3ccc4c(c3)CCO4)c(=O)n(C)c2n1] | (ESI) m/z: 552.2 [M + H]⁺. (400 MHz, DMSO-d₆): δ 8.45-8.32 (m, 4H), 7.85 (m, 1H), 7.46-7.42 (m, 1H), 7.33-7.30 (m, 2H), 7.20 (d, J = 3.6 Hz, 1H), 7.11 (d, J = 3.6 Hz, 1H), 6.52 (m, 1H), 6.05 (s, 1H), 5.65-5.60 (m, 1H), 3.61 (s, 3H), 3.52 (m, 2H), 3.37 (s, 2H), 3.05 (m, 2H), 2.38 (s, 3H), 1.60 (d, J = 7.2 Hz, 3H), 2.12 (s, 6H). |

### Example 12: (R)-2,8-dimethyl-6-morpholin-4-((1-(3-(trifluoromethyl) phenyl) ethyl) amino) pyrido [2,3-d] pyrimidin-7 (8H)-one

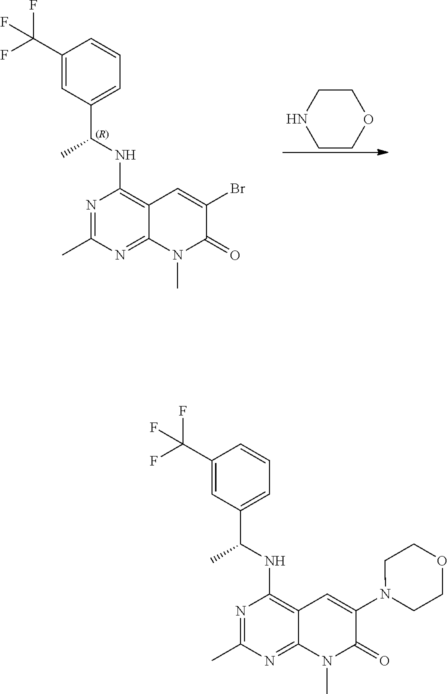

Step 1: under nitrogen protection, (R)-6-bromo-2,8-dimethyl-4-((1-(3-(trifluoromethyl) phenyl) ethyl) amino) pyrido [2,3-d] pyrimidine-7(8H)-one (20.0 mg, 0.04 mmol) and morpholine (7.1 mg, 0.08 mmol), three-generation catalyst Ruphos-Pd-G₃ (3 mg, 0.004 mmol), 2-bicyclohexylphosphine-2′,6′-diisopropoxybiphenyl Ruphos (3 mg, 0.008 mmol), cesium carbonate Cs₂CO₃ (39.2 mg, 0.12 mmol) were dissolved in dioxane (10 mL), and the mixture was heated to 110° C. to react overnight. LCMS detection showed that the reaction was completed.

The reaction solution was concentrated under reduced pressure, and the crude product was separated by HPLC preparation to obtain Example 12 (white solid, 12 mg). LC-MS[M+H]⁺: m/z 448.5. ¹H NMR (400 MHz, CD₃OD): δ 7.74 (s, 1H), 7.69-7.71 (m, 1H), 7.52-7.54 (m, 3H), 5.61-5.64 (m, 1H), 3.87 (t, J=4.4 Hz, 4H), 3.73 (s, 3H), 3.19-3.23 (m, 4H), 2.49 (s, 3H), 1.69 (d, J=7.2 Hz, 3H).

### Example 13: (R)-2,8-dimethyl-6-(tetrahydro-2H-pyran-4-yl) oxy)-4-((1-(3-(trifluoromethyl) phenyl) ethyl) amino) pyrido [2,3-d] pyrimidin-7 (8H)-one

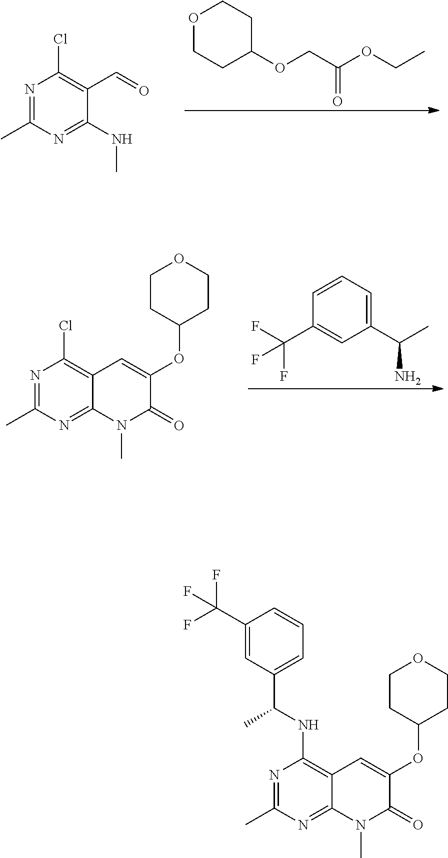 [SMILES: CNc1nc(C)nc(Cl)c1C=O ; CCOC(=O)COC1CCOCC1 ; Cc1nc(Cl)c2cc(OC3CCOCC3)c(=O)n(C)c2n1 ; C[C@@H](N)c1cccc(C(F)(F)F)c1 ; Cc1nc(N[C@H](C)c2cccc(C(F)(F)F)c2)c2cc(OC3CCOCC3)c(=O)n(C)c2n1]

Step 1: LiHMDS (1M, 1.0 mL, 1.0 mmol) was added to a solution of ethyl 2-((tetrahydro-2H-pyran-4-yl) oxy)-acetate (188 mg, 1.0 mmol) in tetrahydrofuran (20 mL) at −78 degree, and the mixture was stirred for 10 minutes. A solution of 4-chloro-2-methyl-6-(methylamino) pyrimidine-5-carbaldehyde (200 mg, 1.0 mmol) in tetrahydrofuran (5 mL) was slowly added, and the reaction solution was gradually warmed to room temperature and reacted overnight. LCMS detection showed that the reaction was completed. Water (50 mL) and ethyl acetate (100 mL) were added to the reaction solution. The separated aqueous phase was extracted twice with ethyl acetate (50 mL), and the organic phases were combined and concentrated under reduced pressure. The crude product was purified by column chromatography (eluent: petroleum ether/ethyl acetate=volume ratio of 5:1), the obtained reaction liquid was concentrated under reduced pressure, and the crude product was separated by HPLC preparation to obtain light yellow oily intermediate compound (32 mg). LC-MS[M+H]: m/z 310.1.

Step 2: the above intermediate compound (20 mg, 0.06 mmol) and (R)-1-(3-(trifluoromethyl) phenyl) ethyl-1-amine (14 mg, 0.06 mmol) were dissolved in acetonitrile (10 mL), DIEA (18 mg, 0.18 mmol) was added, and then the mixture was heated to 100 degrees to react overnight. The reaction solution was concentrated under reduced pressure, and the crude product was purified by HPLC preparation to give the compound of Example 13 (light yellow solid, 9 mg). LC-MS[M+H]⁺: m/z 463.2. ¹H NMR (400 MHz, MeOD-d₄): δ 7.64-7.73 (i, 3H), 7.49-7.51 (m, 2H), 5.61-5.65 (m, 1H), 4.65-4.69 (m, 1H), 3.99-4.04 (i, 2H), 3.73 (s, 3H), 3.56-3.63 (s, 2H), 2.40 (s, 3H), 2.02-2.09 (m, 2H), 1.77-1.86 (i, 2H), 1.65 (d, J=7.2 Hz, 3H).

Referring to the method of Example 8, Examples 14-20 were obtained by the synthesis method wherein 1-methyl-5-(4,4,5,5-tetramethyl-1,3,2-dioxaboran-2-yl)pyridin-2(1H)-one was replaced with different boric acid or boric acid ester, tin reagent as raw material and (R)-1-(3-(trifluoromethyl)phenyl)ethyl-1-amine was replaced with different amine reagents as raw materials.

| No | structure | analysis data(LC-MS and ¹H NMR) |
|---|---|---|
| 14 | 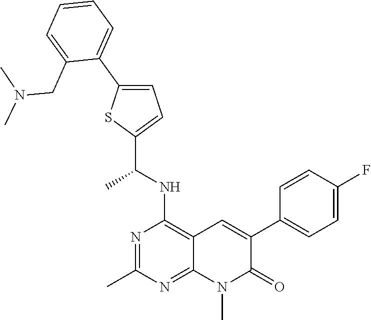 [SMILES: Cc1nc(N[C@H](C)c2ccc(-c3ccccc3CN(C)C)s2)c2cc(-c3ccc(F)cc3)c(=O)n(C)c2n1] | (ESI) m/z: 528.2 [M + H]⁺. (400 MHz, DMSO-d₆): δ 8.38 (m, 1H), 8.32 (d, J = 7.6 Hz, 1H), 7.85 (m, 1H), 7.46-7.42 (m, 1H), 7.33- 7.30 (m, 2H), 7.20 (d, J = 3.6 Hz, 1H), 7.11 (d, J = 3.6 Hz, 1H), 7.24 (m, 2H), 6.05 (s, 1H), 5.87 (m, 1H), 3.57 (s, 2H), 3.42 (s, 3H), 2.38 (s, 3H), 1.60 (d, J = 7.2 Hz, 3H), 2.12 (s, 6H). |
| 15 | 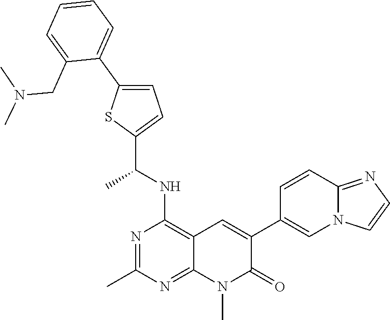 [SMILES: Cc1nc(N[C@H](C)c2ccc(-c3ccccc3CN(C)C)s2)c2cc(-c3ccc4nccn4c3)c(=O)n(C)c2n1] | (ESI) m/z: 550.2 [M + H]⁺. (400 MHz, DMSO-d₆): δ 8.82 (m, 1H), 8.45 (s, 1H), 8.38-8.32 (m, 2H), 7.85 (m, 1H), 7.46-7.30 (m, 5H), 7.20 (d, J = 3.6 Hz, 1H), 7.11 (d, J = 3.6 Hz, 1H), 6.52 (d, J = 8.4 Hz, 1H), 6.05 (s, 1H), 5.60-5.65 (m, 1H), 3.61 (s, 3H), 3.52 (s, 3H), 3.37 (s, 2H), 1.60 (d, J = 7.2 Hz, 3H), 2.12 (s, 6H). |
| 16 | 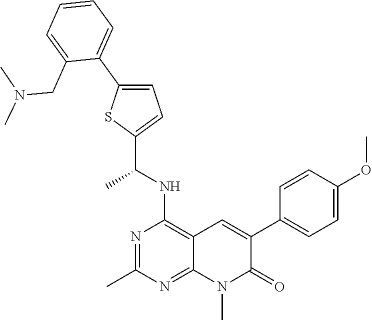 | (ESI) m/z: 540.3 [M + H]⁺. (400 MHz, DMSO-d₆): δ 8.38 (m, 1H), 8.32 (d, J = 7.6 Hz, 1H), 7.85 (m, 1H), 7.52 (d, J = 7.2 Hz, 2H), 7.35 (s, 1H), 7.33-7.30 (m, 2H), 7.20 (d, J = 3.6 Hz, 1H), 7.11 (d, J = 3.6 Hz, 1H), 6.88 (d, J = 7.2 Hz, 2H), 5.61-5.64 (m, 1H), 3.85 (s, 3H), 3.57 (s, 2H), 3.42 (s, 3H), 2.38 (s, 3H), 1.60 (d, J = 7.2 Hz, 3H), 2.12 (s, 6H). |
| 17 | 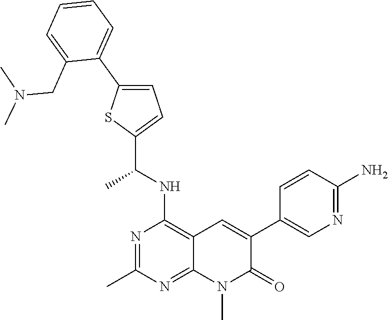 [SMILES: Cc1nc(N[C@H](C)c2ccc(-c3ccccc3CN(C)C)s2)c2cc(-c3ccc(N)nc3)c(=O)n(C)c2n1] | (ESI) m/z: 526.2 [M + H]⁺. (400 MHz, DMSO-d₆): δ 8.82 (m, 1H), 8.45 (s, 1H), 8.38-8.32 (m, 2H), 7.85 (m, 1H), 7.46-7.30 (m, 4H), 7.20 (d, J = 3.6 Hz, 1H), 7.11 (d, J = 3.6 Hz, 1H), 6.52 (d, J = 8.4 Hz, 1H), 6.05 (s, 1H), 5.60-5.65 (m, 1H), 3.61 (s, 3H), 3.52 (s, 3H), 3.37 (s, 2H), 1.60 (d, J = 7.2 Hz, 3H), 2.12 (s, 6H). |
| 18 | 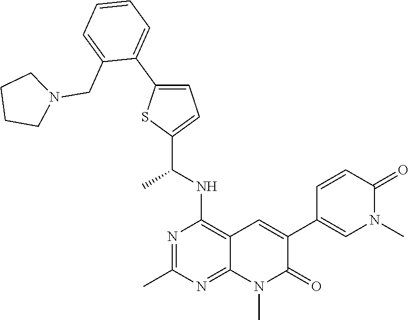 [SMILES: Cc1nc(N[C@H](C)c2ccc(-c3ccccc3CN3CCCC3)s2)c2cc(-c3ccc(=O)n(C)c3)c(=O)n(C)c2n1] | (ESI) m/z: 567.3 [M + H]⁺. ¹H NMR (400 MHz, DMSO-d₆): δ 8.36 (s, 1H), 8.30 (m, 1H), 7.95 (m, 1H), 7.61-7.63 (m, 1H), 7.47-7.52 (m, 3H), 7.15 (d, J = 3.6 Hz, 1H), 6.99 (d, J = 3.6 Hz, 1H), 6.61 (m, 1H), 5.96-6.01 (m, 1H), 4.54 (s, 2H), 3.76 (s, 3H), 3.64 (s, 3H), 3.48 (m, 2H), 2.98 (m, 2H), 2.54 (s, 3H), 1.78-1.92 (m, 4H), 1.77 (d, J = 6.8 Hz, 3H). |
| 19 | 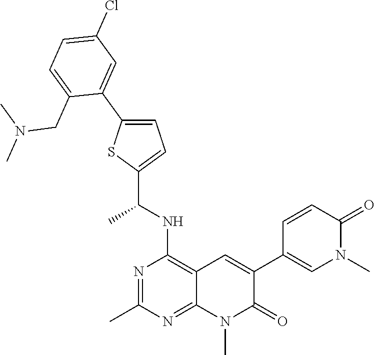 | (ESI) m/z: 575.2 [M + H]⁺. (400 MHz, DMSO-d₆): δ 8.82 (m, 1H), 8.45 (s, 1H), 8.37-8.35 (m, 1H), 7.85 (m, 1H), 7.46-7.30 (m, 2H), 7.20 (d, J = 3.6 Hz, 1H), 7.11 (d, J = 3.6 Hz, 1H), 6.52 (m, 1H), 6.05 (s, 1H), 5.65-5.60 (m, 1H), 3.61 (s, 3H), 3.52 (s, 3H), 3.37 (s, 2H), 2.38 (s, 3H), 1.65 (d, J = 7.2 Hz, 3H), 2.12 (s, 6H). |
| 20 | 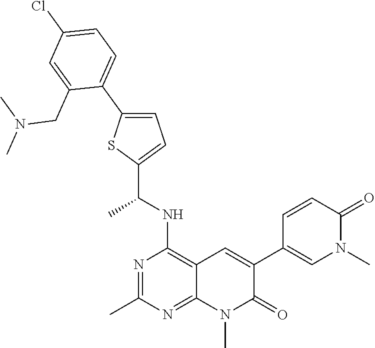 | (ESI) m/z: 575.2 [M + H]⁺. (400 MHz, DMSO-d₆): δ 8.82 (m, 1H), 8.35 (s, 1H), 8.33-8.25 (m, 1H), 7.85 (m, 1H), 7.46-7.30 (m, 2H), 7.20 (d, J = 3.6 Hz, 1H), 7.11 (d, J = 3.6 Hz, 1H), 6.52 (m, 1H), 6.05 (s, 1H), 5.60-5.65 (m, 1H), 3.61 (s, 3H), 3.52 (s, 3H), 3.37 (s, 2H), 2.38 (s, 3H), 1.63 (d, J = 7.2 Hz, 3H), 2.12 (s, 6H). |

### Example 21: (R)-2-methyl-6-(tetrahydro-2H-pyran-4-yl)-N-(1-(3-(trifluoromethyl)phenyl) ethyl)imidazo (1′,2′:1,6) pyrido [2,3-d] pyrimidin-4-amino

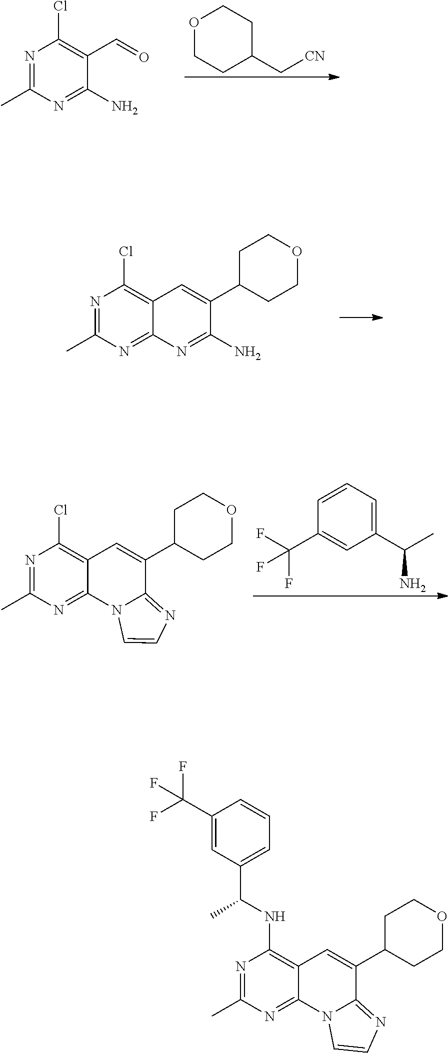

Step 1: 4-amino-6-chloro-2-methyl pyrimidine-5-carbaldehyde (100 mg, 0.6 mmol) and 2-(tetrahydro-2H-pyran-4-yl) acetonitrile (225 mg, 1.8 mmol) were dissolved in tetrahydrofuran (20 mL) under nitrogen protection, and LiHMDS (1.0 mol/L, 1.8 mL, 1.8 mmol) was added and stirred for 10 minutes at −78 degrees. The reaction solution was slowly warmed to room temperature and stirred overnight. LC-MS detection showed that the reaction was basically completed, after water (40 mL) being added to the reaction solution, the mixture was extracted with ethyl acetate (80 mL) for three times. The organic phase was concentrated, and the crude product was purified by silica gel column chromatography (dichloromethane/methanol=10:1) to give a yellow solid product (35 mg). LC-MS (ESI) m/z: 279.1 [M+H]⁺.

Step 2: The above intermediate (20 mg, 0.07 mmol) and chloroacetaldehyde (8 mg, 0.1 mmol) were dissolved in a mixed solvent of ethanol (10 mL) and water (1 mL), saturated sodium bicarbonate solution NaHCO₃ (13 mg, 0.15 mmol) was added, and the reaction mixture was heated to 100 degrees and stirred overnight. LC-MS detection showed that the reaction was completed, the reaction mixture was concentrated, water (30 mL) was added, and the mixture was extracted with ethyl acetate (60 mL) for three times. The combined organic phase was concentrated and purified by HPLC preparation to give a white solid compound (6 mg). LC-MS (ESI) m/z: 303.2 [M+H]⁺.

Step 3: the above intermediate product (6.0 mg, 0.02 mmol) and (R)-1-(3-(trifluoromethyl) phenyl) ethyl-1-amine (4.3 mg, 0.02 mmol) were dissolved in dimethyl sulfoxide DMSO (5 mL), N,N-diisopropylethylamine (DIEA)(10 mg, 0.08 mmol) and potassium fluoride KF (5.2 mg, 0.08 mmol) were added, the reaction solution was heated to 120 degrees and reacted overnight. LC-MS detection showed that the reaction was completed. The reaction solution was concentrated under reduced pressure, and the crude product was separated by HPLC preparation to obtain the target product (light yellow solid, 2 mg). LC-MS [M+H]⁺: m/z 456.1. ¹H NMR (400 MHz, CD₃OD): δ 8.64 (s, 1H), 8.47 (s, 1H), 8.00 (s, 1H), 7.79-7.74 (m, 2H), 7.55-7.52 (m, 2H), 5.75-5.73 (i, 1H), 4.16-4.13 (m, 2H), 3.72-3.69 (i, 2H), 3.39-3.37 (m, 1H), 2.58 (s, 3H), 2.03-1.99 (m, 4H), 1.72 (d, J=7.2 Hz, 3H).

Referring to the same synthesis method as in Example 21, the following Examples 22-25 were obtained by the synthesis method wherein various 2-substituted acetonitrile was used as starting materials instead of 2-(tetrahydro-2H-pyran-4-yl) acetonitrile and different amine reagents was used as starting materials instead of (R)-1-(3-(trifluoromethyl) phenyl) ethyl-1-amine:

| No | structure | analysis data |
|---|---|---|
| 22 | 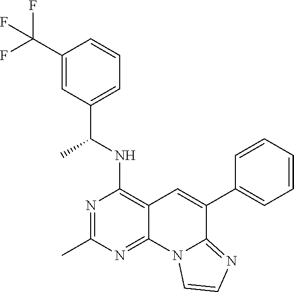 [SMILES: Cc1nc(N[C@H](C)c2cccc(C(F)(F)F)c2)c2cc(-c3ccccc3)c3nccn3c2n1] | LC-MS (ESI) m/z: 448.2 [M + H]⁺. ¹H NMR (400 MHz, CD₃OD): δ 8.71 (d, J = 2.4 Hz, 1H), 8.62 (s, 1H), 7.95 (d, J = 2.0 Hz 1H), 7.80- 7.74 (m, 4H), 7.66-7.52 (m, 5H), 5.76-5.72 (m, 1H), 2.62 (s, 3H), 1.70 (d, J = 7.2 Hz, 3H). |
| 23 | 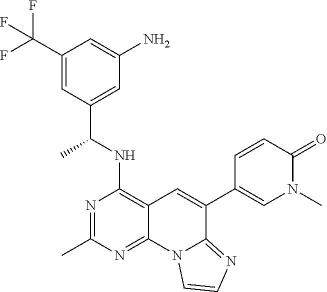 [SMILES: Cc1nc(N[C@H](C)c2cc(N)cc(C(F)(F)F)c2)c2cc(-c3ccc(=O)n(C)c3)c3nccn3c2n1] | LC-MS (ESI) m/z: 494.2 [M + H]⁺. ¹H NMR (400 MHz, MeOD-d₄): 8.63 (s, 1H), 8.43 (s, 1H), 8.17-8.13 (m, 2H), 7.63 (s, 1H), 7.00- 6.95 (m, 2H), 6.81 (s, 1H), 6.69-6.72 (m, 1H), 5.62-5.64 (m, 1H), 3.70 (s, 3H), 2.57 (s, 3H), 1.63-1.66 (m, 3H). |
| 24 | 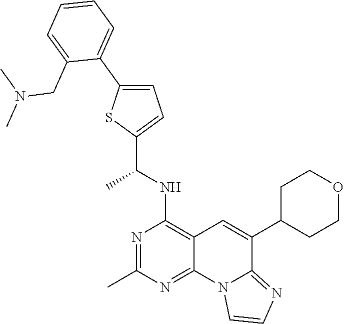 [SMILES: Cc1nc(N[C@H](C)c2ccc(-c3ccccc3CN(C)C)s2)c2cc(C3CCOCC3)c3nccn3c2n1] | LC-MS (ESI) m/z: 527.3 [M + H]⁺. ¹H NMR (400 MHz, CD₃OD): δ 8.35 (m, 1H), 8.12 (s, 1H), 7.43-7.37 (m, 4H), 7.18-7.10 (m, 3H), 5.73-5.75 (m, 1H), 4.13-4.16 (m, 2H), 3.72-3.69 (m, 2H), 3.47-3.39 (m, 3H), 2.58 (s, 3H), 2.12 (s, 6H), 1.99-2.03 (m, 4H), 1.72 (d, J = 7.2 Hz, 3H). |
| 25 | 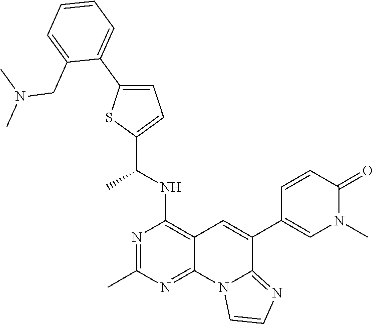 | LC-MS (ESI) m/z: 550.2 [M + H]⁺. 1H NMR (400 MHz, MeOD-d₄): δ 8.65 (s, 1H), 8.43-8.35 (m, 2H), 8.13-8.17 (m, 2H), 7.43-7.37 (m, 4H), 7.07-6.95 (m, 2H), 6.69-6.72 (m, 1H), 5.62-5.64 (m, 1H), 3.70 (s, 3H), 3.38 (s, 2H), 2.57 (s, 3H), 2.12 (s, 6H), 1.63-1.66 (m, 3H). |

Referring to the methods of Examples 8 and 12, examples 26-29 were obtained by the synthesis method wherein 1-methyl-5-(4,4,5,5-tetramethyl-1,3,2-dioxaboran-2-yl) pyridin-2(1H)-one was replaced with different boric acids or boric acid esters, tin reagents as starting materials and (R)-1-(3-(trifluoromethyl) phenyl) ethyl-1-amine was replaced with different amine reagents as raw materials:

| No | structure | analysis data(LC-MS and ¹H NMR) |
|---|---|---|
| 26 | 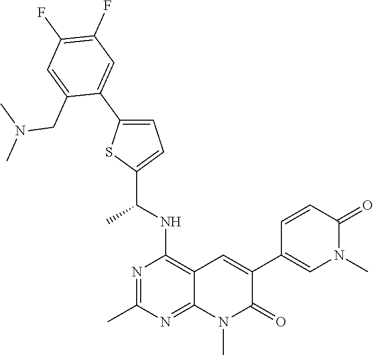 | (ESI) m/z: 577.2 [M + H]⁺. (400 MHz, DMSO-d₆): δ 7.58 (m, 1H), 7.30 (s, 1H), 7.26 (m, 1H), 7.20 (d, J = 3.6 Hz, 1H), 7.11 (d, J = 3.6 Hz, 1H), 7.14 (m, 1H), 6.95-6.83 (m, 2H), 6.05 (s, 1H), 5.87 (m, 1H), 3.57 (s, 2H), 3.42 (s, 3H), 2.38 (s, 3H), 1.60 (d, J = 7.2 Hz, 3H), 2.12 (s, 6H). |
| 27 | 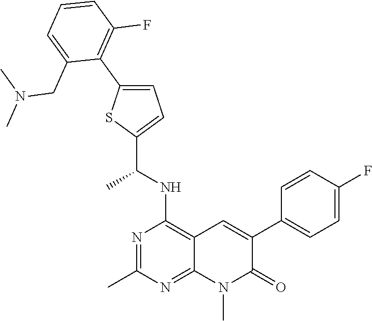 [SMILES: Cc1nc(N[C@H](C)c2ccc(-c3c(F)cccc3CN(C)C)s2)c2cc(-c3ccc(F)cc3)c(=O)n(C)c2n1] | (ESI) m/z: 544.2 [M + H]⁺. ¹H NMR (400 MHz, DMSO-d₆): δ 8.34 (s, 1H), 7.73-7.62 (m, 3H), 7.48-7.52 (m, 2H), 7.13-7.17 (m, 3H), 6.98 (d, J = 3.6 Hz, 1H), 5.97-5.99 (m, 1H), 4.52 (s, 2H), 3.77 (s, 3H), 2.54 (s, 3H), 2.15 (s, 6H), 1.76 (d, J = 7.2 Hz, 3H). |
| 28 | 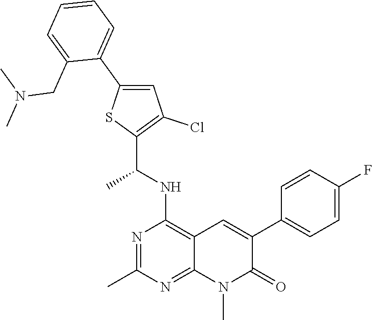 [SMILES: Cc1nc(N[C@H](C)c2sc(-c3ccccc3CN(C)C)cc2Cl)c2cc(-c3ccc(F)cc3)c(=O)n(C)c2n1] | (ESI) m/z: 562.2 [M + H]⁺. ¹H NMR (400 MHz, DMSO-d₆): δ 8.32 (s, 1H), 7.73-7.62 (m, 4H), 7.48-7.52 (m, 3H), 7.13-7.17 (m, 2H), , 5.97-5.99 (m, 1H), 4.50 (s, 2H), 3.75 (s, 3H), 2.55 (s, 3H), 2.12 (s, 6H), 1.72 (d, J = 7.2 Hz, 3H). |
| 29 | 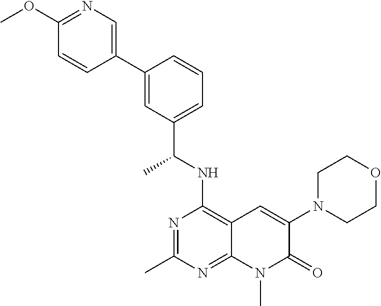 | (ESI) m/z: 569.2 [M + H]⁺. (400 MHz, DMSO-d₆) δ 8.53 (d, J = 7.9 Hz, 1H), 8.46 (m, 1H), 8.00 (m, 1H), 7.71 (s, 1H), 7.54 (m, 1H), 7.44 (m, 2H), 6.91 (d, J = 8.6 Hz, 1H), 5.86 (s, 1H), 5.61-5.64 (m, 1H), 3.89 (s, 3H), 3.19-3.23 (m, 4H), 2.46 (s, 3H), 1.69 (d, J = 7.2 Hz, 3H), 1.56-1.63 (m, 4H). |

### Example 30: (R)-N-(1-(3-amino-5-(trifluoromethyl) phenyl) ethyl)-6-(2,3-dihydrobenzofuran-5-yl)-2-methylimidazole [1′,2′:1,6] pyridine [2,3-d] pyrimidin-4-amine

Referring to the same method of Example 21, example 30 was obtained by the synthesis method wherein benzodihydrofuran acetonitrile was used as raw material instead of 2-(tetrahydro-2H-pyran-4-yl) acetonitrile and (R)-3-(1-aminoethyl)-5-(trifluoromethyl) aniline was used as raw material instead of (R)-1-(3-(trifluoromethyl) phenyl) ethyl-1-amine.

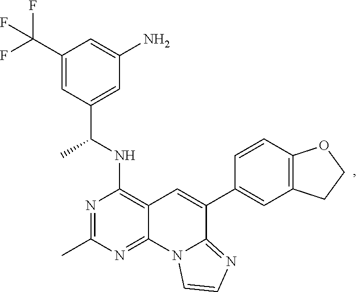 [SMILES: Cc1nc(N[C@H](C)c2cc(N)cc(C(F)(F)F)c2)c2cc(-c3ccc4c(c3)CCO4)c3nccn3c2n1]

LC-MS (ESI) m/z: 505.2 [M+H]⁺. ¹H NMR (400 MHz, CD₃OD): δ 8.62 (s, 1H), 8.32 (s, 1H), 7.75-7.52 (m, 5H), 7.31 (s, 1H), 6.95 (s, 1H), 5.76-5.72 (m, 1H), 3.45 (m, 2H), 2.62 (s, 3H), 2.09 (m, 2H), 1.70 (d, J=7.2 Hz, 3H).

### Example 31: (R)-4-((1-(3-amino-5-(trifluoromethyl) phenyl) ethyl) amino)-2,8-dimethyl-6-morpholinopyridine [2,3-d] opyrimidin-7 (8H)-one

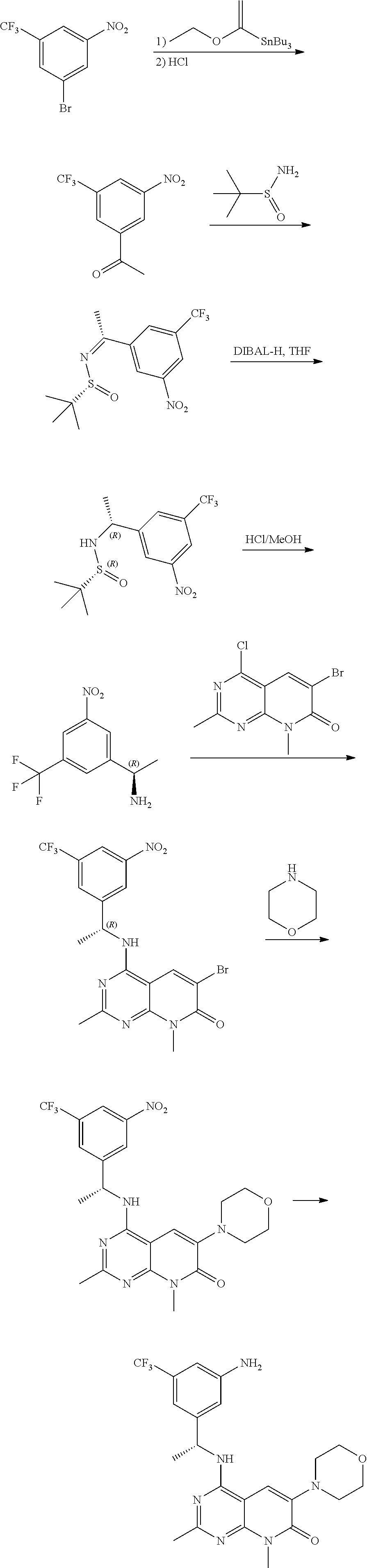

Step 1: At room temperature, tributyl-1-(ethoxyethylene) tin (20 g, 55.6 mmol) and tetrakistriphenylphosphine palladium (321 mg, 5.6 mmol) were added to 1-bromo-3-nitro-5-trifluoromethylbenzene (10 g, 37.0 mmol) in 1,4-dioxane (100 mL). The reaction mixture was reacted at 100 degrees for 4 hours. LCMS detection showed that the reaction was basically completed. The reaction solution was diluted with ethyl acetate (40 mL), filtered through celite, and the filtrate was concentrated under reduced pressure to give 11 g of the crude intermediate compound as a yellow solid. The crude compound was dissolved in acetonitrile (400 mL), aqueous hydrochloric acid (2M, 80 mL, 160 mmol) was added, and then the mixture was heated to 80° C. to react for 3 hours. LCMS detection showed that the reaction was basically completed. After most of the solvent was removed by concentration under reduced pressure, the reaction mixture was diluted with ethyl acetate (200 mL) and washed twice with brine (100 mL). The separated organic phase was dried over anhydrous sodium sulfate, and the filtrate was concentrated under reduced pressure. The obtained crude product was isolated by HPLC preparation to give a light yellow solid intermediate compound (5.3 g). LCMS (ESI) m/z: 234.0 [M+H]⁺. ¹H NMR (400 MHz, DMSO-d6) δ 8.86 (s, 1H), 8.71 (s, 1H), 8.63 (s, 1H), 2.78 (s, 3H).

Step 2: Under nitrogen protection, tetraethyl titanate (9.3 g, 40.9 mmol) and (R)-(+)-tert-butylsulfinamide (2.5 g, 20.4 mmol) were added to the above intermediate compound (5.3 g, 22.7 mmol) in tetrahydrofuran (40 mL). The reaction mixture was reacted at 70 degrees for 16 hours. LCMS detection showed that the reaction was completed. The reaction solution was diluted with ethyl acetate (200 mL), and the organic phase was washed with brine (50 mL) twice. The separated organic phase was dried over anhydrous sodium sulfate, and the filtrate was concentrated under reduced pressure. The obtained crude product was purified by silica gel column chromatography (eluent: petroleum ether/ethyl acetate=4:1) to give a light yellow solid compound (6.0 g). LC-MS (ESI) m/z: 336.9 [M+H]⁺. ¹H NMR (400 MHz, DMSO-d6) δ 8.85 (s, 1H), 8.65 (s, 1H), 8.55 (s, 1H), 2.86 (s, 3H), 1.26 (s, 9H).

Step 3: DIBAL-H (35 mL, 35 mmol) was slowly added to a solution of the above intermediate compound (5.8 g, 17.2 mmol) in tetrahydrofuran (100 mL) below −60° C. The temperature of the reaction solution was gradually raised to room temperature and the reaction was continued at this temperature for 16 hours. LCMS detection showed that the reaction was basically completed. After quenching by adding methanol (10 mL), the reaction mixture was diluted with ethyl acetate (100 mL). The reaction mixture was filtered through celite, and the filtrate was concentrated under reduced pressure. The resulting crude product was purified by silica gel column chromatography (eluent: petroleum ether/ethyl acetate=4:1) to give the intermediate compound as a light yellow solid (2.9 g). LCMS (ESI) m/z: 338.9 [M+H]⁺. ¹H NMR (400 MHz, DMSO) δ 8.63 (s, 1H), 8.38 (s, 1H), 8.30 (s, 1H), 6.08 (d, J=8.6 Hz, 1H), 4.83-4.57 (m, 1H), 1.46 (d, J=6.9 Hz, 3H), 1.14 (s, 9H).

Step 4: At room temperature, a solution of hydrogen chloride (2M, 10 mL, 20 mmol) in methanol was added to the above intermediate compound (2.9 g, 8.57 mmol) in methanol (20 mL), and the reaction mixture was reacted at 80 degrees for 2 hours. LCMS detection showed that the reaction was basically completed. The reaction solution was concentrated under reduced pressure to remove most of the solvent, saturated sodium carbonate solution (50 mL) was added to the residue, and the mixture was extracted twice with dichloromethane (100 mL). The combined organic phases were dried over anhydrous sodium sulfate, the filtrate was concentrated under reduced pressure, and the obtained crude product was purified by silica gel column chromatography (eluent: dichloromethane/methanol=10:1) to give the intermediate compound as a light yellow solid (2 g). LCMS (ESI) m/z: 235.1 [M+H]⁺.

Step 5: 6-bromo-4-chloro-2,8-dimethylpyridine [2,3-d] pyrimidin-7 (8H)-one (15 mg, 0.05 mmol) and (R)-1-(3-nitro-5-(trifluoromethyl) phenyl) ethyl-1-amine (14 mg, 0.06 mmol) were dissolved in NMP (5 mL), DIEA (19.2 mg, 0.15 mmol) was added, and the mixture was reacted at 100 degrees overnight. LCMS detection showed that the reaction was completed. The reaction solution was diluted with ethyl acetate (30 mL), washed with water (20 mL) twice, the separated organic phase was concentrated under reduced pressure, and the obtained crude product was purified by HPLC preparation to give a white solid intermediate product (15 mg). LC-MS[M+H]⁺: m/z 486.0/488.0.

Step 6: Under nitrogen protection, the above intermediate compound (15.0 mg, 0.03 mmol), morpholine (6.1 mg, 0.06 mmol), Ruphos-Pd-G₃ (3 mg, 0.004 mmol), Ruphos (3 mg, 0.008 mmol), Cs₂CO₃ (39.2 mg, 0.12 mmol) were dissolved in dioxane (10 mL), and the mixture was heated to 110° C. to react overnight. The reaction solution was concentrated under reduced pressure, and the obtained crude product was purified by HPLC preparation to give a white solid intermediate product (5 mg). LC-MS[M+H]⁺: m/z 493.2. ¹H NMR (400 MHz, CD₃OD): δ 8.58 (s, 1H), 8.37 (s, 1H), 8.19 (s, 1H), 7.51 (s, 1H), 5.62-5.64 (m, 1H), 3.86-3.89 (m, 4H), 3.71 (s, 3H), 3.16-3.19 (m, 4H), 2.39 (s, 3H), 1.71 (d, J=7.2 Hz, 3H).

Step 7: The above intermediate compound (10.0 mg, 0.02 mmol) was dissolved in a mixed solution of MeOH/THF/H₂O (10 mL/10 mL/10 mL), zinc powder (13.0 mg, 0.2 mmol) and ammonium chloride (11.2 mg, 0.02 mmol) were added, and the reaction solution was stirred overnight at room temperature. The reaction solution was diluted with methanol (20 mL), filtered through celite, and the filtrate was concentrated under reduced pressure. The obtained crude product was separated by HPLC preparation to obtain the compound of Example 31 (gray solid, 1.0 mg). LC-MS[M+H]⁺: m/z 463.3. ¹H NMR (400 MHz, CD₃OD): δ 7.54 (s, 1H), 7.08 (s, 1H), 7.04 (s, 1H), 6.90 (s, 1H), 5.53-5.55 (m, 1H), 3.85-3.88 (m, 4H), 3.73 (s, 3H), 3.12-3.24 (m, 4H), 2.46 (s, 3H), 1.63 (d, J=6.8 Hz, 3H).

Referring to the methods of Examples 12 and 31, examples 32-47 were obtained by the synthesis method wherein morpholine was replaced with different amino groups as raw materials and (R)-1-(3-(trifluoromethyl) phenyl) ethyl-1-amine or (R)-1-(3-nitro-5-(trifluoromethyl) phenyl) ethyl-1-amine was re laced with different benzylamine reagents as raw materials.

| No | structure | analysis data(LC-MS and ¹H-NMR) |
|---|---|---|
| 32 | 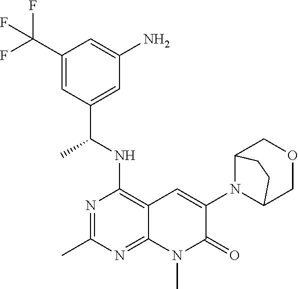 [SMILES: Cc1nc(N[C@H](C)c2cc(N)cc(C(F)(F)F)c2)c2cc(N3C4CCC3COC4)c(=O)n(C)c2n1] | (ESI) m/z: 489.2 [M + H]⁺. (400 MHz, CD₃OD): δ 7.39 (s, 1H), 7.12 (s, 1H), 7.07 (s, 1H), 6.96 (s, 1H), 5.51-5.53 (m, 1H), 4.48 (s, 2H), 3.89-3.93 (m, 2H), 3.74 (s, 3H), 3.55-3.58 (m, 2H), 2.50 (s, 3H), 2.02-2.05 (m, 4H), 1.30 (d, J = 1.2 Hz, 3H). |
| 33 | 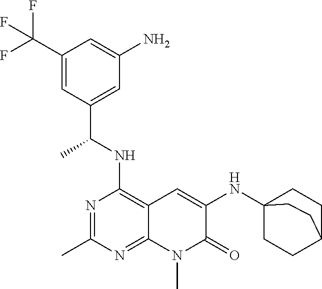 [SMILES: Cc1nc(N[C@H](C)c2cc(N)cc(C(F)(F)F)c2)c2cc(NC34CCC(CC3)CC4)c(=O)n(C)c2n1] | (ESI) m/z: 501.3 [M + H]⁺. (400 MHz, MeOD-d₄): δ 7.01 (s, 1H), 6.95 (s, 1H), 6.93 (s, 1H), 6.80 (s, 1H), 5.56-5.53 (m, 1H), 3.75 (s, 3H), 2.41 (s, 3H), 1.63-1.51 (m, 7H), 1.35-0.97 (m, 9H). |
| 34 | 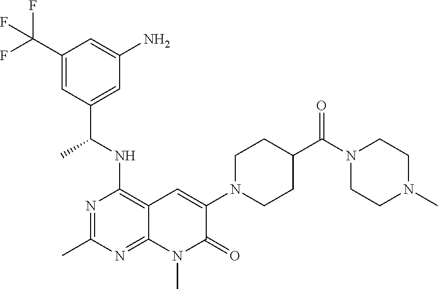 [SMILES: Cc1nc(N[C@H](C)c2cc(N)cc(C(F)(F)F)c2)c2cc(N3CCC(C(=O)N4CCN(C)CC4)CC3)c(=O)n(C)c2n1] | (ESI) m/z: 587.3 [M + H]⁺. (400 MHz, CD₃OD): δ 7.56 (s, 1H), 7.02 (s, 1H), 6.99 (s, 1H), 6.84 (s, 1H), 5.51-5.58 (m, 1H), 3.72-3.79 (m, 2H), 3.72 (s, 3H), 3.30-3.35 (m, 4H), 3.22-3.29 (m, 2H), 2.95 (s, 3H), 2.90-2.95 (m, 3H), 2.68-2.85 (m, 2H), 2.45 (s, 3H), 1.92- 2.06 (m, 3H), 1.82-1.89 (m, 2H), 1.61 (d, J = 7.2 Hz, 3H). |
| 35 | 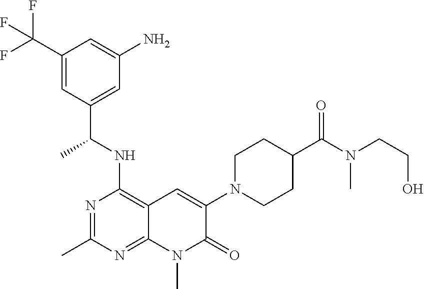 [SMILES: Cc1nc(N[C@H](C)c2cc(N)cc(C(F)(F)F)c2)c2cc(N3CCC(C(=O)N(C)CCO)CC3)c(=O)n(C)c2n1] | (ESI) m/z: 562.3 [M + H]⁺. (400 MHz, CD₃OD): δ 7.56 (s, 1 H), 7.02 (s, 1 H), 6.99 (s, 1H), 6.84 (s, 1H), 5.63-5.65 (m, 1H), 3.74- 3.85 (m, 2H), 3.69-3.73 (m, 6H), 3.57-3.59 (m, 1H), 3.49-3.51 (m, 1H), 3.30-3.31 (m, 1H), 2.84-2.97 (m, 4H), 2.40 (s, 3H), 1.71- 2.04 (m, 5H), 1.30 (d, J = 14.0 Hz, 3H). |
| 36 | 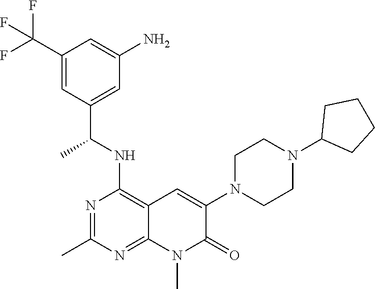 [SMILES: Cc1nc(N[C@H](C)c2cc(N)cc(C(F)(F)F)c2)c2cc(N3CCN(C4CCCC4)CC3)c(=O)n(C)c2n1] | (ESI)m/z: 530.3 [M + H]⁺. (400 MHz, CD₃OD): δ 7.56 (s, 1H), 7.02 (s, 1H), 6.99 (s, 1H), 6.84 (s, 1H), 5.51-5.58 (m, 1H), 3.72-3.79 (m, 2H), 3.72 (s, 3H), 3.30-3.35 (m, 4H), 3.22-3.29 (m, 2H), 2.95 (s, 3H), 2.90-2.95 (m, 3H), 2.68-2.85 (m, 2H), 2.45 (s, 3H), 1.92- 2.06 (m, 3H), 1.82-1.89 (m, 2H), 1.61 (d, J = 7.2 Hz, 3H). |
| 37 | 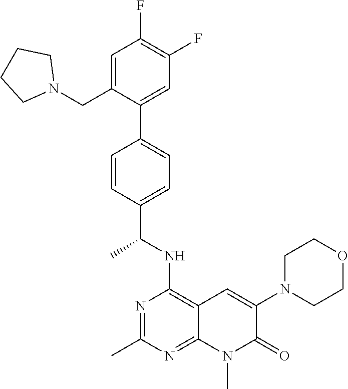 [SMILES: Cc1nc(N[C@H](C)c2ccc(-c3cc(F)c(F)cc3CN3CCCC3)cc2)c2cc(N3CCOCC3)c(=O)n(C)c2n1] | (ESI) m/z: 575.3 [M + H]⁺. (400 MHz, CD₃OD): δ 7.74 (s, 1H), 8.07 (m, 1H), 7.65 (d, J = 8.1 Hz, 2H), 7.47-7.37 (m, 2H), 6.20 (s, 1H), 5.61-5.64 (m, 1H), 4.30 (s, 2H), 3.87 (t, J = 4.4 Hz, 4H), 3.73 (s, 3H), 3.19-3.23 (m, 4H), 2.43-2.51 (m, 7H), 1.69 (d, J = 7.2 Hz, 3H), 1.56-1.63 (m, 4H). |
| 38 | 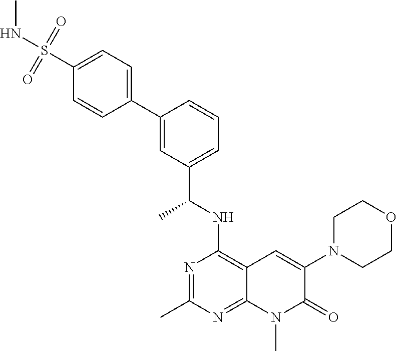 [SMILES: CNS(=O)(=O)c1ccc(-c2cccc([C@@H](C)Nc3nc(C)nc4c3cc(N3CCOCC3)c(=O)n4C)c2)cc1] | (ESI) m/z: 549.2 [M + H]⁺. (400 MHz, CD₃OD): δ 7.94 (d, J = 8.4 Hz, 2H), 7.71-7.74 (m, 3H), 7.43-7.55 (m, 4H), 5.65-5.70 (m, 1H), 3.85-3.87 (m, 4H), 3.77-3.73 (m, 3H), 3.72 (s, 3H), 3.16- 3.18 (m, 4H), 2.46 (s, 3H), 1.70 (d, J = 6.8 Hz, 3H). |
| 39 | 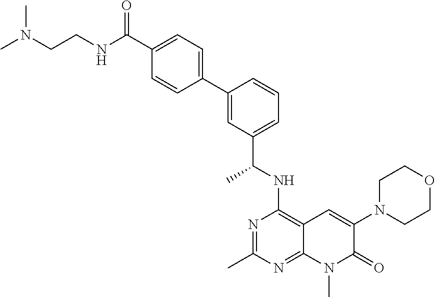 [SMILES: Cc1nc(N[C@H](C)c2cccc(-c3ccc(C(=O)NCCN(C)C)cc3)c2)c2cc(N3CCOCC3)c(=O)n(C)c2n1] | (ESI) m/z: 570.3 [M + H]⁺. (400 MHz, CD₃OD): δ 7.94 (d, J = 8.4 Hz, 2H), 7.71-7.74 (m, 3H), 7.55-7.43 (m, 4H), 5.70-5.65 (m, 1H), 3.87-3.85 (m, 4H), 3.79-3.76 (m, 2H), 3.72 (s, 3H), 3.41- 3.38 (m, 2H), 3.18-3.16 (m, 4H), 2.99 (s, 6H), 2.46 (s, 3H), 1.70 (d, J = 6.8 Hz, 3H). |
| 40 | 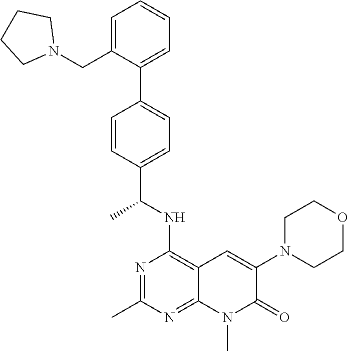 [SMILES: Cc1nc(N[C@H](C)c2ccc(-c3ccccc3CN3CCCC3)cc2)c2cc(N3CCOCC3)c(=O)n(C)c2n1] | (ESI) m/z: 539.3 [M + H]⁺. (400 MHz, CD₃OD): δ 7.54-7.47 (m, 4H), 7.34-7.24 (m, 4H), 7.22-7.20 (m, 1H), 5.69-5.67 (m, 1H), 3.88-3.85 (m, 4H), 3.73 (s, 3H), 3.67 (s, 2H), 3.17-3.12 (m, 4H), 2.44 (s, 3H), 2.44-2.41 (m, 4H), 1.69-1.66 (m, 7H). |
| 41 | 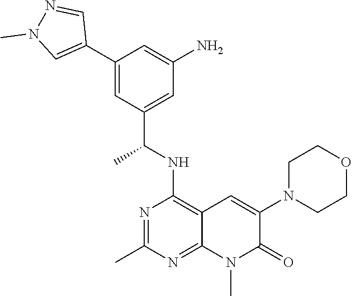 | (ESI) m/z: 575.3 [M + H]⁺. ¹H NMR (400 MHz, CD₃OD): δ 8.85 (s, 1H), 8.20 (s, 1H), 7.59 (s, 1H), 7.18 (s, 1H), 7.08 (s, 1H), 6.90 (s, 1H), 5.57-5.51 (m, 1H), 3.94 (s, 3H), 3.89-3.83 (m, 4H), 3.73 (s, 3H), 3.24-3.12 (m, 4H), 2.46 (s, 3H), 1.63 (d, J = 6.8 Hz, 3H). |
| 42 | 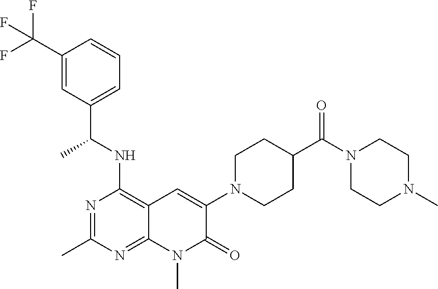 [SMILES: Cc1nc(N[C@H](C)c2cccc(C(F)(F)F)c2)c2cc(N3CCC(C(=O)N4CCN(C)CC4)CC3)c(=O)n(C)c2n1] | (ESI) m/z: 572.3 [M + H]⁺. (400 MHz, CD₃OD): δ 7.74-7.71 (m, 3H), 7.55-7.49 (m, 2H), 5.65-5.60 (m, 1H), 4.56-4.42 (m, 2H), 3.79-3.76 (m, 2H), 3.72 (s, 3H), 3.54-3.45 (m, 4H), 3.19-3.14 (m, 3H), 3.04-2.85 (m, 6H), 2.46 (s, 3H), 2.07-1.89 (m, 4H), 1.67 (d, J = 7.2 Hz, 3H). |
| 43 | 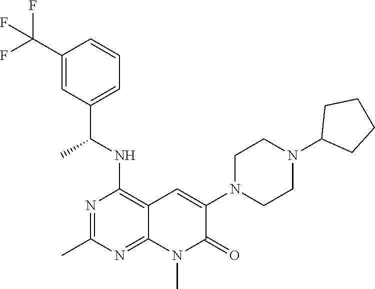 [SMILES: Cc1nc(N[C@H](C)c2cccc(C(F)(F)F)c2)c2cc(N3CCN(C4CCCC4)CC3)c(=O)n(C)c2n1] | (ESI) m/z: 515.3 [M + H]⁺. (400 MHz, CD₃OD): δ 7.73(s, 1H), 7.68 (m, 1H), 7.61 (s, 1H), 7.52 (m, 2H), 5.51-5.58 (m, 1H), 3.72-3.79 (m, 2H), 3.72 (s, 3H), 3.30-3.35 (m, 4H), 3.22-3.29 (m, 2H), 2.95 (s, 3H), 2.90-2.95 (m, 3H), 2.68-2.85 (m, 2H), 2.45 (s, 3H), 1.92- 2.06 (m, 3H), 1.82-1.89 (m, 2H), 1.61 (d, J = 7.2Hz, 3H). |
| 44 | 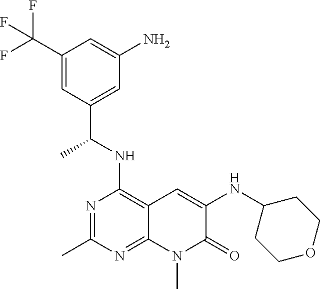 [SMILES: Cc1nc(N[C@H](C)c2cc(N)cc(C(F)(F)F)c2)c2cc(NC3CCOCC3)c(=O)n(C)c2n1] | (ESI) [M + H]⁺: m/z 477.2. ¹H NMR (400 MHz, MeOD-d₄): δ 7.00 (s, 1H), 6.96 (s, 1H), 6.94 (s, 1H), 6.79 (s, 1H), 5.53-5.56 (m, 1H), 3.98-4.02 (m, 2H), 3.75 (s, 3H), 3.55-3.62 (m, 3H), 2.41 (s, 3H), 2.05-2.09 (m, 2H), 1.53-1.61 (m, 5H). |
| 45 | 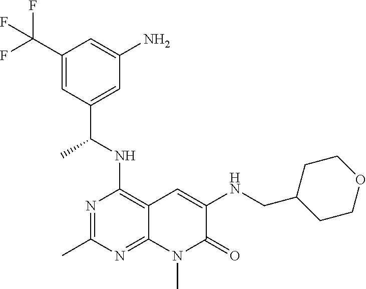 [SMILES: Cc1nc(N[C@H](C)c2cc(N)cc(C(F)(F)F)c2)c2cc(NCC3CCOCC3)c(=O)n(C)c2n1] | (ESI) [M + H]⁺: m/z 491.2. ¹H NMR (400 MHz, DMSO-d₆): δ 7.65 (d, J = 8.0 Hz, 1H), 6.91 (s, 1H), 6.86 (s, 1H), 6.83 (s, 1H), 6.69 (s, 1H), 5.52-5.54 (m, 3H), 3.85-3.86 (m, 3H), 3.68 (s, 3H), 3.34- 3.38 (m, 4H), 3.06-3.08 (m, 2H), 2.34 (s, 3H), 1.66-1.69 (m, 2H), 1.54-1.65 (m, 2H), 1.27 (d, J = 12.8 Hz, 3H). |
| 46 | 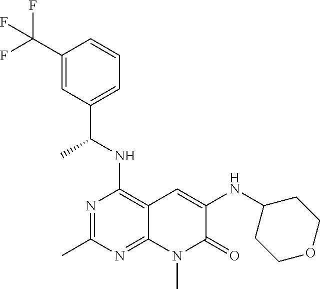 [SMILES: Cc1nc(N[C@H](C)c2cccc(C(F)(F)F)c2)c2cc(NC3CCOCC3)c(=O)n(C)c2n1] | (ESI) [M + H]⁺: m/z 462.2. ¹H NMR (400 MHz, MeOD-d₄): δ 7.69- 7.75 (m, 2H), 7.54-7.57 (m, 2H), 7.01 (s, 1H), 5.61-5.63 (m, 1H), 3.98-4.03 (m, 2H), 3.78 (s, 3H), 3.54-3.67 (m, 3H), 2.52 (s, 3H), 2.03-2.07 (m, 2H), 1.72 (d, J = 7.2 Hz, 3H), 1.59-1.61 (m, 2H). |
| 47 | 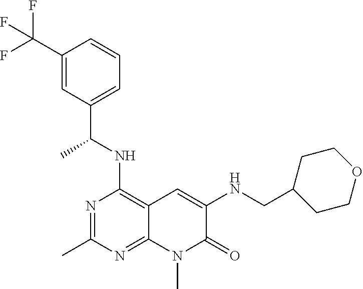 [SMILES: Cc1nc(N[C@H](C)c2cccc(C(F)(F)F)c2)c2cc(NCC3CCOCC3)c(=O)n(C)c2n1] | (ESI) [M + H]⁺: m/z 476.2. ¹H NMR (400 MHz, MeOD-d₄): δ 7.69- 7.75 (m, 2H), 7.54-7.58 (m, 2H), 6.95 (s, 1H), 5.60-5.63 (m, 1H), 3.95-3.99 (m, 2H), 3.78 (s, 3H), 3.41-3.47 (m, 2H), 3.17 (d, J = 7.2 Hz, 2H), 2.51 (s, 3H), 1.98-2.02 (m, 1H), 1.71-1.78 (m, 5H), 1.33-1.43 (m, 2H). |

### Example 48: (R)-4-((1-(3-amino-5-(ethyltrifluoromethyl) phenyl) ethyl) amino)-2,8-dimethyl-6-((tetrahydro-2H-pyran-4-yl) oxo) pyridine [2,3-d] pyrimidin-7 (8H)-one

The compound of Example 48 was prepared by following the synthetic method of Example 13 using (R)-3-(1-aminoethyl)-5-(trifluoromethyl) aniline instead of (R)-1-(3-(trifluoromethyl) phenyl) ethyl-1-amine.

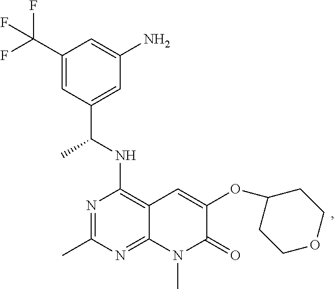 [SMILES: Cc1nc(N[C@H](C)c2cc(N)cc(C(F)(F)F)c2)c2cc(OC3CCOCC3)c(=O)n(C)c2n1]

LC-MS[M+H]⁺: m/z 478.2. ¹H NMR (400 MHz, MeOD-d4): δ 7.64-7.73 (m, 2H), 7.49-7.51 (m, 2H), 5.61-5.65 (m, 1H), 4.65-4.69 (m, 1H), 3.99-4.04 (m, 2H), 3.73 (s, 3H), 3.56-3.63 (m, 2H), 2.40 (s, 3H), 2.02-2.09 (m, 2H), 1.77-1.86 (m, 2H), 1.65 (d, J=7.2 Hz, 3H).

### Example 49: (R)-6-(4-Fluorophenyl)-2,8-dimethyl-4-((1-(5-(2-(pyrrolin-1-ylmethyl) phenyl) thiophen-2-yl) ethyl) amino) pyridine [2,3-d] pyrimidin-7 (8H)-one

Referring to the method of Example 8, the compound of Example 49 was obtained by the synthesis method wherein 1-methyl-5-(4,4,5,5-tetramethyl-1,3,2-dioxaboron-2-yl) pyridin-2(1H)-one was replaced with p-fluorophenylboronic acid as the raw material and (R)-1-(3-(trifluoromethyl) phenyl) ethyl-1-amine was replaced with (R)-1-(5-(2-(pyrrol-1-ylmethyl) phenyl thiophen-2-1 ethyl-1-amine hydrochloride as the starting material.

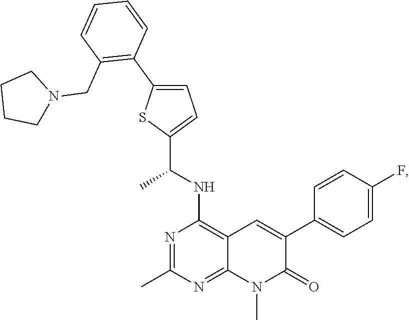 [SMILES: Cc1nc(N[C@H](C)c2ccc(-c3ccccc3CN3CCCC3)s2)c2cc(-c3ccc(F)cc3)c(=O)n(C)c2n1]

LC-MS[M+H]⁺: m/z 554.2. ¹H NMR (400 MHz, DMSO-d₆): δ 8.34 (s, 1H), 7.73-7.69 (m, 2H), 7.62-7.60 (m, 1H), 7.52-7.48 (m, 3H), 7.17-7.13 (m, 3H), 6.98 (d, J=3.6 Hz, 1H), 5.99-5.97 (m, 1H), 4.52 (s, 2H), 3.77 (s, 3H), 3.40-3.36 (m, 2H), 2.98-2.96 (m, 2H), 2.54 (s, 3H), 1.89-1.87 (m, 4H), 1.76 (d, J=7.2 Hz, 3H).

Referring to the methods of Examples 12 and 31 examples 50-55 were obtained by the synthesis method wherein morpholine was replaced with different amino groups as raw materials and (R)-1-(3-(trifluoromethyl) phenyl) ethyl-1-amine or (R)-1-(3-nitro-5-(trifluoromethyl) phenyl) ethyl-1-amine was re laced with different benzylamine reagents as raw materials;

| No | structure | analysis data(LC-MS and ¹H-NMR) |
|---|---|---|
| 50 | 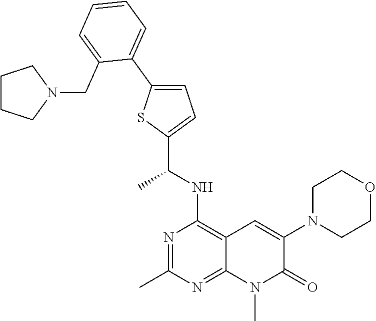 [SMILES: Cc1nc(N[C@H](C)c2ccc(-c3ccccc3CN3CCCC3)s2)c2cc(N3CCOCC3)c(=O)n(C)c2n1] | (ESI) [M + H]⁺: m/z 545.3. ¹H NMR (400 MHz, MeOD-d₄): δ 7.53- 7.48 (m, 2H), 7.37-7.27 (m, 3H), 7.04 (m, 1H), 6.98 (m, 1H), 5.98 (m, 1H), 3.87-3.84 (m, 4H), 3.74 (s, 6H), 3.15-3.12 (m, 4H), 2.51 (s, 7H), 1.75 (d, J = 7.2 Hz, 3H), 1.71-1.68 (m, 4H). |
| 51 | 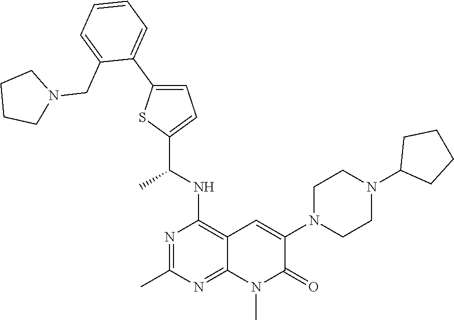 [SMILES: Cc1nc(N[C@H](C)c2ccc(-c3ccccc3CN3CCCC3)s2)c2cc(N3CCN(C4CCCC4)CC3)c(=O)n(C)c2n1] | (ESI) [M + H]⁺: m/z 612.3.(400 MHz, MeOD-d₄): δ 7.57-7.45 (m, 5H), 7.10 (d, J = 3.2 Hz, 1H), 6.97 (d, J = 3.2 Hz, 1H), 6.84 (s, 1H), 5.51-5.58 (m, 1H), 3.72-3.79 (m, 2H), 3.72 (s, 3H), 3.30-3.35 (m, 4H), 3.22-3.29 (m, 2H), 2.95 (s, 3H), 2.90-2.95 (m, 3H), 2.68-2.85 (m, 2H), 2.45 (s, 3H), 1.92-2.06 (m, 3H), 1.82-1.89 (m, 2H), 1.61 (d, J = 7.2 Hz, 3H). |
| 52 | 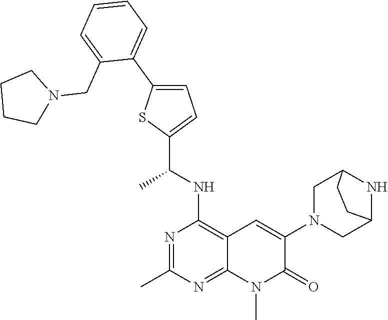 [SMILES: Cc1nc(N[C@H](C)c2ccc(-c3ccccc3CN3CCCC3)s2)c2cc(N3CC4CCC(C3)N4)c(=O)n(C)c2n1] | (ESI) [M + H]⁺: m/z 570.3. (400 MHz, DMSO-d₆): δ 7.65-7.62 (m, 1H), 7.54-7.48 (m, 4H), 7.13 (d, J = 3.2 Hz, 1H), 6.98 (d, J = 3.2 Hz, 1H), 5.97-5.95 (m, 1H), 4.54 (s, 2H), 4.14 (s, 2H), 3.83-3.79 (m, 2H), 3.74 (s, 3H), 3.39 (m, 2H), 3.10-3.06 (m, 2H), 2.99-2.97 (m, 2H), 2.51 (s, 3H), 2.36-2.33 (m, 2H), 2.12-2.09 (m, 2H), 1.91 (m, 4H), 1.77 (d, J = 7.2 Hz, 3H). |
| 53 | 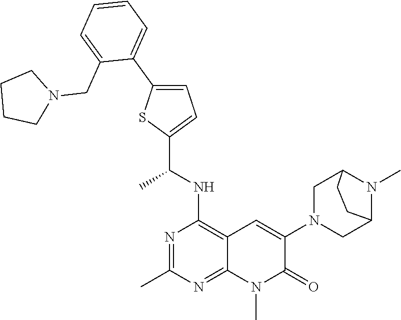 [SMILES: Cc1nc(N[C@H](C)c2ccc(-c3ccccc3CN3CCCC3)s2)c2cc(N3CC4CCC(C3)N4C)c(=O)n(C)c2n1] | (ESI) [M + H]⁺: m/z 584. (400 MHz, DMSO-d₆): δ 7.65-7.62 (m, 1H), 7.54-7.48 (m, 4H), 7.13 (d, J = 3.2 Hz, 1H), 6.98 (d, J = 3.2 Hz, 1H), 5.97-5.95 (m, 1H), 4.54 (s, 2H), 4.14 (s, 2H), 3.83-3.79 (m, 2H), 3.74 (s, 3H), 3.39 (m, 2H), 3.10-3.06 (m, 2H), 2.99-2.97 (m, 2H), 2.51 (s, 3H), 2.36-2.29 (m, 5H), 2.13-2.08 (m, 2H), 1.93 (m, 4H), 1.77 (d, J = 7.2 Hz, 3H). |
| 54 | 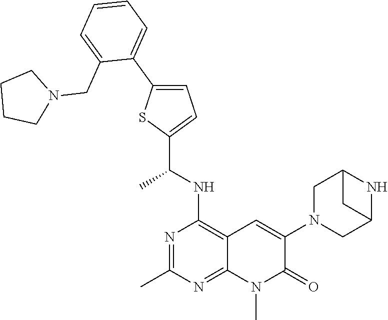 [SMILES: Cc1nc(N[C@H](C)c2ccc(-c3ccccc3CN3CCCC3)s2)c2cc(N3CC4CC(C3)N4)c(=O)n(C)c2n1] | (ESI) [M + H]⁺: m/z 556.3. (400 MHz, DMSO-d₆): δ 7.65-7.62 (m, 1H), 7.54-7.48 (m, 4H), 7.13 (d, J = 3.2 Hz, 1H), 6.98 (d, J = 3.2 Hz, 1H), 5.97-5.95 (m, 1H), 4.54 (s, 2H), 4.14 (s, 2H), 3.83-3.79 (m, 2H), 3.74 (s, 3H), 3.35 (m, 2H), 3.10-3.06 (m, 2H), 2.99-2.97 (m, 2H), 2.51 (s, 3H), 2.36-2.33 (m, 2H), 2.12-2.09 (m, 2H), 1.91 (m, 2H), 1.72 (d, J = 7.2 Hz, 3H). |
| 55 | 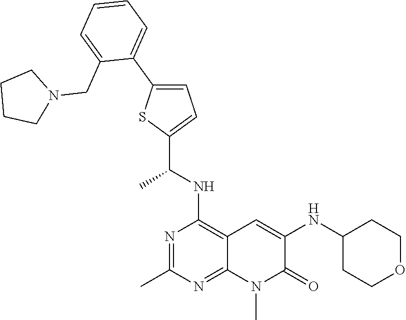 [SMILES: Cc1nc(N[C@H](C)c2ccc(-c3ccccc3CN3CCCC3)s2)c2cc(NC3CCOCC3)c(=O)n(C)c2n1] | (ESI) [M + H]⁺: m/z 559.3. ¹H NMR (400 MHz, MeOD-d₄): δ 7.49- 7.47 (m, 1H), 7.37-7.27 (m, 3H), 7.03 (d, J = 3.2 Hz, 1H), 6.98 (d, J = 3.6 Hz, 1H), 6.95 (s, 1H), 5.98-5.96 (m, 1H), 4.02-3.94 (m, 2H), 3.77 (s, 3H), 3.74 (s, 2H), 3.65-3.46 (m, 3H), 2.50 (s, 7H), 2.11- 2.06 (m, 2H) 1.76 (d, J = 6.8 Hz, 3H), 1.69-1.66 (m, 4H), 1.58- 1.46 (m, 2H). |

### Example 56: (R)-4-((1-(2′-((dimethylamino) methyl))-[1,1′-biphenyl]-4-yl) ethyl) amino-2,8 dimethyl-6-morpholinopyridine [2,3-d] opyrimidin-7 (8H)-one

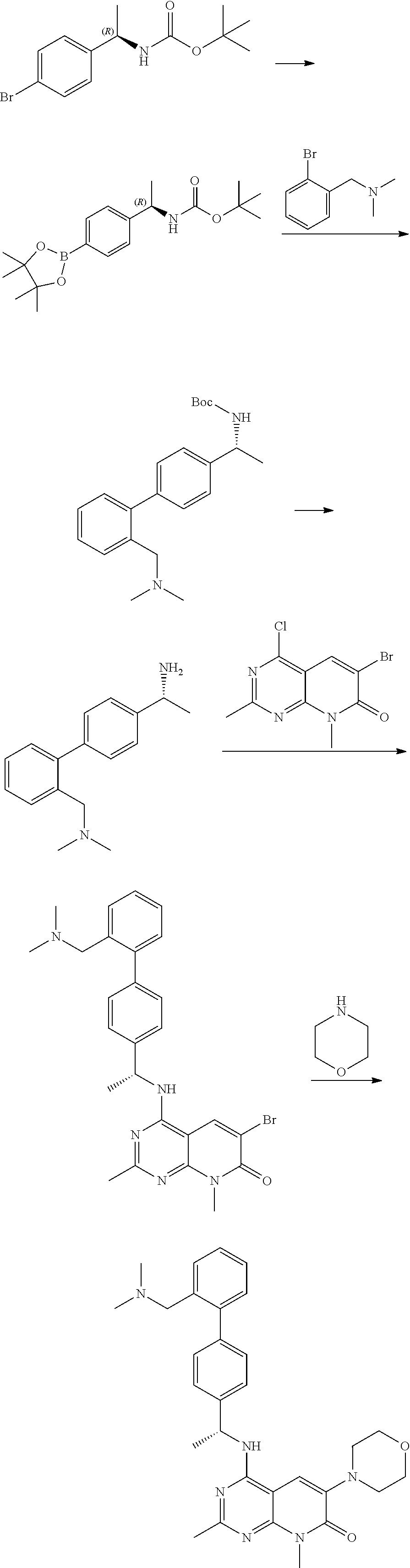

Step 1: under nitrogen protection, to a solution of compound tert-butyl (R)-1-((4-bromophenyl) ethyl) carbamate (200 mg, 0.67 mmol) in 1,4-dioxane (20 mL), bis(pinacolato)diboron (200 mg, 0.8 mmol), potassium acetate (130 mg, 1.3 mmol) and [1,1′-bis (diphenylphosphino) ferrocene] palladium dichloride PdCl₂(dppf) (21 mg, 0.03 mmol) were added successively. The reaction mixture was stirred at 90 degrees overnight, and LCMS detection showed that the reaction was completed. The reaction solution was concentrated under reduced pressure, and the obtained crude product was purified by silica gel column chromatography (eluent: petroleum ether/ethyl acetate=10:1) to give light yellow oily intermediate compound (300 mg). LC-MS [M-56+H]⁺: m/z 292.3.

Step 2: the above intermediate compound (70 mg, 0.2 mmol) and 1-(2-bromobenzene)-N,N-dimethylmethanamide (43 mg, 0.2 mmol) were dissolved in a mixed solution of dioxane/water (Dioxane/H₂O) (6 mL/1 mL), potassium phosphate (85.0 mg, 0.4 mmol) and [1,1′-bis (di-tert-butylphosphine) ferrocene palladium (II) dichloride Pd(dtbpf)Cl₂ (12.0 mg, 0.02 mmol) were added, the reaction solution was heated to 100 degrees and stirred overnight. LCMS detection showed that the reaction was completed, the reaction solution was concentrated under reduced pressure, and the obtained crude product was separated to obtain a yellow oily liquid intermediate compound (60 mg) by HPLC preparation. LC-MS[M+H]⁺: m/z 355.3.

Step 3: the above intermediate compound (40.0 mg, 0.11 mmol) was dissolved in methanol (5 mL), and HCl (gas)/methanol solution (2 mL) was added. The reaction solution was stirred at room temperature for 2 hours, and LCMS detection showed that the reaction was completed. The reaction solution was concentrated under reduced pressure to give the crude intermediate compound as a yellow solid (30 mg). LC-MS[M+H]⁺: m/z 255.2.

Step 4: the above intermediate compound (26.0 mg, 0.1 mmol) and 6-bromo-4-chloro-2,8-dimethylpyridine[2,3-d]pyrimidin-7 (8H)-one (30.0 mg, 0.1 mmol) were dissolved in 1-methylpyrrolidone NMP (5 mL), N,N-diisopropylethylamine (38.9 mg, 0.3 mmol) was added, the reaction solution was heated to 110 degrees and stirred overnight. The reaction solution was diluted with ethyl acetate (30 mL), washed with water (10 mL) twice, the separated organic phase was concentrated under reduced pressure, and the obtained crude product was purified by HPLC preparation to obtain a light yellow solid intermediate compound (20.0 mg). LC-MS[M+H]⁺: m/z 508.2.

Step 5: Under nitrogen protection, the above intermediate compound (20.0 mg, 0.04 mmol) and morpholine (10.0 mg, 0.12 mmol) were dissolved in dioxane (6 mL), cesium carbonate (39.0 mg, 0.12 mmol), Ruphos-Pd-G₃ (3.0 mg, 0.004 mmol) and Ruphos (2.0 mg, 0.004 mmol) were added, the reaction solution was heated to 100 degrees and stirred overnight. The reaction solution was filtered through celite, and the filtrate was concentrated under reduced pressure. The obtained crude product was separated by HPLC preparation to obtain the compound of Example 56 (light yellow solid, 1.30 mg). LC-MS[M+H]⁺: m/z 513.3. ¹H NMR (400 MHz, CD₃OD): δ 7.63-7.51 (m, 6H), 7.39-7.37 (m, 1H), 7.31 (d, J=8.0 Hz, 1H), 5.72-5.69 (m, 1H), 4.38 (s, 2H), 3.89-3.86 (m, 4H), 3.73 (s, 3H), 3.23-3.18 (m, 4H), 2.61 (s, 6H), 2.48 (s, 3H), 1.70 (d, J=7.2 Hz, 3H).

Referring to the method of Example 56, Examples 57-60 were obtained by the synthesis method that 1-(2-bromobenzene)-N,N-dimethylmethylamine was replaced with different aryl brominated compounds as raw materials:

| No | structure | analysis data |
|---|---|---|
| 57 | 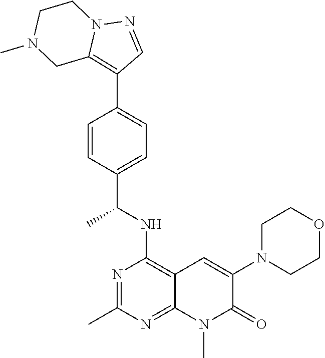 [SMILES: Cc1nc(N[C@H](C)c2ccc(-c3cnn4c3CN(C)CC4)cc2)c2cc(N3CCOCC3)c(=O)n(C)c2n1] | LC-MS [M + H]⁺: m/z 515.3. ¹H NMR (400 MHz, MeOD-d₄): δ 10.20 (s, 1H), 7.97 (s, 1H), 7.49 (d, J = 8.0 Hz, 2H), 7.43 (d, J = 8.0 Hz, 1H), 6.88 (s, 1H), 5.73-5.68 (m, 1H), 3.88-3.85 (m, 4H), 3.27 (s, 2H), 3.23 (s, 3H), 2.97-2.92 (m, 4H), 2.85-2.76 (m, 4H), 2.45 (s, 3H), 2.36 (s, 3H), 1.67 (d, J = 7.2 Hz, 3H). |
| 58 | 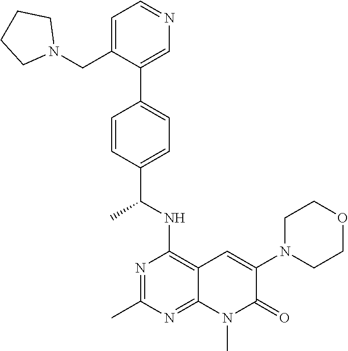 [SMILES: Cc1nc(N[C@H](C)c2ccc(-c3cnccc3CN3CCCC3)cc2)c2cc(N3CCOCC3)c(=O)n(C)c2n1] | LC-MS [M + H]⁺: m/z 540.3. ¹H NMR (400 MHz, MeOD-d₄): δ 8.32 (s, 1H), 8.02 (m, 1H), 7.54-7.47 (m, 3H), 7.34-7.22 (m, 5H), 5.67-5.69 (m, 1H), 3.85-3.88 (m, 4H), 3.73 (s, 3H), 3.67 (s, 2H), 3.17-3.12 (m, 4H), 2.44 (s, 3H), 2.44-2.41 (m, 4H), 1.69- 1.65 (m, 7H) |
| 59 | 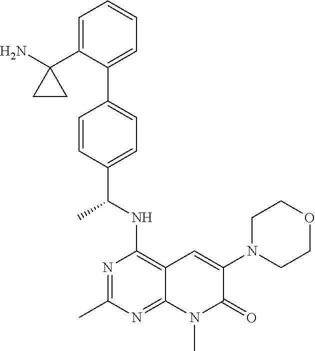 [SMILES: Cc1nc(N[C@H](C)c2ccc(-c3ccccc3C3(N)CC3)cc2)c2cc(N3CCOCC3)c(=O)n(C)c2n1] | LC-MS [M + H]⁺: m/z 511.3. ¹H NMR (400 MHz, CD₃OD): δ 7.63-7.51 (m, 6H), 7.39-7.37 (m, 1H), 7.31 (d, J = 8.0 Hz, 1H), 5.72-5.68 (m, 1H), 3.89-3.86 (m, 4H), 3.73 (s, 3H), 3.23-3.18 (m, 4H), 2.48 (s, 3H), 1.70 (d, J = 7.2 Hz, 3H), 0.8 (m, 2H), 0.5 (m, 2H). |
| 60 | 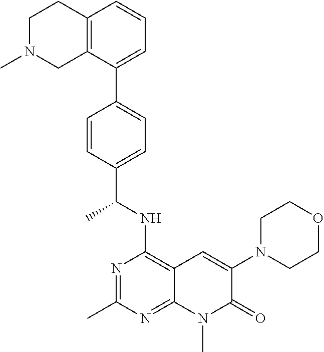 [SMILES: Cc1nc(N[C@H](C)c2ccc(-c3cccc4c3CN(C)CC4)cc2)c2cc(N3CCOCC3)c(=O)n(C)c2n1] | LC-MS [M + H]⁺: m/z 525.3. ¹H NMR (400 MHz, MeOD-d₄): δ 7.54 (s, 1H), 7.49 (d, J = 8.0 Hz, 2H), 7.23-7.19 (m, 3H), 7.13 (d, J = 8.0 Hz, 1H), 7.00 (d, J = 7.2 Hz, 1H), 5.73-5.68 (m, 1H), 3.88-3.85 (m, 4H), 3.73 (s, 3H), 3.47 (s, 2H), 3.17-3.12 (m, 4H), 3.04-3.01 (m, 2H), 2.80-2.76 (m, 2H), 2.45 (s, 3H), 2.36 (s, 3H), 1.67 (d, J = 7.2 Hz, 3H). |

### Example 61: (R)-4-((1-(3-fluoro-5-(2-(pyrrolidin-1-ylmethyl) phenyl) thiophen-2-yl) ethyl) amino)-2,8-dimethyl-6-morpholinopyridine [2,3-d] opyrimidin-7 (8H)-one

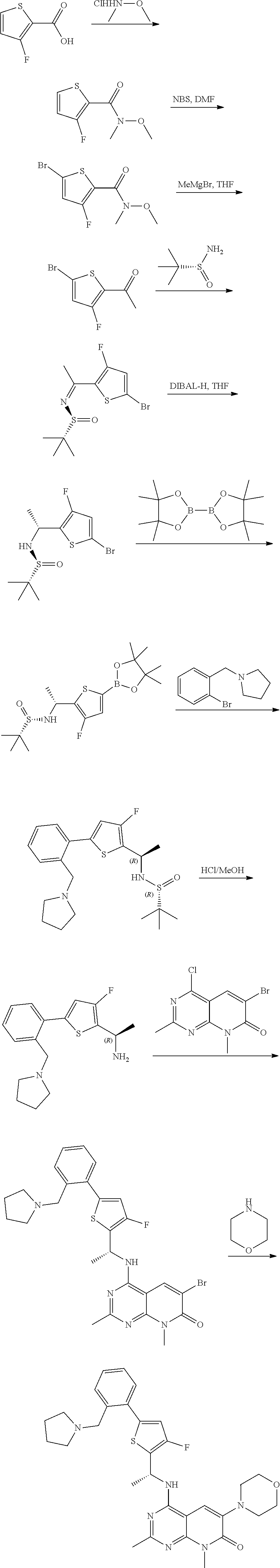

Step 1: 1-Ethyl-(3-dimethylaminopropyl) carbodiimide hydrochloride (13.85 g, 72.3 mmol) was added to a solution of 3-fluorothiophene-2-carboxylic acid (4.8 g, 32.85 mmol) and N,O-dimethylhydroxylamine hydrochloride (7.05 g, 72.3 mmol) in pyridine (30 mL) at room temperature, and the reaction mixture was reacted at room temperature for 16 hours. The reaction solution was concentrated under reduced pressure to remove the solvent, and the obtained crude product was purified by silica gel column chromatography (eluent: petroleum ether/ethyl acetate=4:1) to give the intermediate compound as a yellow solid (6.1 g). LCMS (ESI) m/z: 190.0 [M+H]⁺.

Step 2: Under nitrogen protection, N-bromosuccinimide (17.5 g, 98.31 mmol) was added to the above intermediate compound (6.1 g, 32.8 mmol) in N,N-dimethylformamide (100 mL). The reaction mixture was heated to 60° C. and reacted at this temperature for 16 hours. The mixture was diluted with ethyl acetate (500 mL) and then washed with saturated brine (100 mL) for three times. The separated organic phase was dried over anhydrous sodium sulfate, and the filtrate was concentrated under reduced pressure. The resulting crude product was subjected to silica gel column chromatography (eluent: ethyl acetate/petroleum ether=4:1) to obtain a brown solid intermediate compound (3.5 g). LCMS (ESI) m/z: 269.8 [M+H]⁺1H NMR (400 MHz, DMSO-d6)δ 7.42 (s, 1H), 3.74 (s, 3H), 3.22 (s, 3H).

Step 3: under nitrogen protection, methyl magnesium bromide (30 mL, 31.7 mmol) was added to a solution of the above intermediate compound (3.4 g, 12.7 mmol) in tetrahydrofuran (50 mL) at 0° C., and the reactant was further reacted at 0° C. for 1 hour. After LCMS detection showed that the reaction of the raw materials was completed, ammonium chloride solution (200 mL) was added to quench the reaction. The reaction solution was extracted twice with ethyl acetate (150 mL), the combined organic phases were dried over anhydrous sodium sulfate, the filtrate was concentrated under reduced pressure, and the obtained crude product was subjected to HPLC preparation to obtain a brown oily intermediate compound (2.2 g). LCMS (ESI) m/z: 224.8 [M+H]⁺ 0.1H NMR (400 MHz, DMSO-d6)δ 8.23 (s, 1H), 2.54 (s, 3H).

Step 4: Under nitrogen protection, tetraethyl titanate (3.94 g, 17.2 mmol) was added to the above compound (2.2 g, 9.5 mmol), (R)-(+) tert-butyl sulfinamide (1.05 g, 8.64 mmol) in tetrahydrofuran (30 mL), and the reaction mixture was heated to 70° C. and reacted at this temperature for 16 hours. After the reaction solution was cooled to room temperature, brine (50 mL) was added, and stirring was continued for 10 minutes. The reaction mixture was filtered through celite, and the filtrate was extracted twice with ethyl acetate (100 mL). The combined organic phase was dried over anhydrous sodium sulfate, and the filtrate was concentrated under reduced pressure. The obtained crude product was purified by silica gel column chromatography (eluent: ethyl acetate/petroleum ether=4:1) to give the intermediate compound (1.8 g) as a brown solid. LCMS (ESI) m/z: 326.0 [M+H]⁺ 0.1H NMR (400 MHz, DMSO-d6)δ 7.47 (s, 1H), 2.65 (d, J=2.4 Hz, 3H), 1.18 (s, 9H).

Step 5: Under cooling at −78° C., DIBAL-H (15 mL, 14.1 mmol) was added to the above intermediate compound (1.8 g, 5.5 mmol) in tetrahydrofuran (30 mL), the reaction mixture was slowly warmed to room temperature and reacted at this temperature for 16 hours, and LCMS detection showed that the reaction was basically completed. Methanol (20 mL) was added to quench the reaction, the mixture was concentrated under reduced pressure to remove most of the solvent, then the residue was diluted with methanol (200 mL), filtered through celite, and the filtrate was concentrated under reduced pressure. The obtained crude product was purified by silica gel column chromatography (eluent: ethyl acetate/petroleum ether=4:1) to give a brown solid compound (1.4 g). LCMS (ESI) m/z: 329.9 [M+H]⁺. 1H NMR (400 MHz, DMSO-d6)δ 7.19 (s, 1H), 5.89 (d, J=6.6 Hz, 1H), 4.66 (m, 1H), 1.45 (d, J=6.8 Hz, 3H), 1.10 (s, 9H).

Step 6: Under nitrogen protection, PdCl₂(dppf) (21 mg, 0.03 mmol) was added to a solution of the above intermediate compound (700 mg, 2.1 mmol), bis(pinacolato)diboron (813 mg, 3.2 mmol) and potassium acetate (365 mg, 3.7 mmol) in 1,4-dioxane (50 mL). The reaction solution was heated to 100 degrees and reacted for 10 hours. LCMS detection showed that the reaction was basically completed. The reaction solution was concentrated under reduced pressure, and the crude product was purified by silica gel column chromatography (eluent: petroleum ether/ethyl acetate=10:1) to give the intermediate compound as a yellow oil (510 mg). LC-MS[M+H]⁺: m/z 376.2.

Step 7: Under nitrogen protection, the above intermediate compound (200 mg, 0.5 mmol) and 1-(2-bromophenyl) pyrrole (130 mg, 0.54 mmol), Pd(dppf)Cl₂ (50 mg, 0.1 mmol), potassium phosphate (127 mg, 0.6 mmol) were dissolved in dioxane/H₂O (18 mL/3 mL), and the mixture was heated to 90 degrees to react overnight. The reaction solution was concentrated under reduced pressure, and the crude product was separated by HPLC preparation to obtain a white solid product (163 mg). LC-MS[M+H]⁺: m/z 409.2.

Step 7: a solution of hydrochloric acid in methanol (2M, 10 mL, 20 mmol) was added to the above intermediate compound (163 mg, 0.4 mmol) in methanol (10 mL). The reactants were reacted at room temperature for 2 hours. LCMS detection showed that the reaction was almost completed. The mixture was concentrated under reduced pressure to give crude intermediate compound (110 mg) as a brown solid. LCMS (ESI) m/z: 304.2 [M+H]⁺.

Step 8: the above intermediate compound (30 mg, 0.1 mmol) and 6-bromo-4-chloro-2,8-dimethylpyridine [2,3-d] pyrimidin-7 (8H)-one (30.0 mg, 0.1 mmol) were dissolved in NMP (5 mL), N,N-diisopropylethylamine (38.9 mg, 0.3 mmol) was added, the reaction solution was heated to 110 degrees and stirred overnight. The reaction solution was diluted with ethyl acetate (30 mL), washed with water (10 mL) twice, the separated organic phase was concentrated under reduced pressure, and the obtained crude product was purified by HPLC preparation to obtain a light yellow solid intermediate compound (40 mg). LC-MS[M+H]⁺: m/z 556.1/558.1.

Step 9: Under nitrogen protection, the above intermediate compound (40 mg, 0.07 mmol) and morpholine (10.0 mg, 0.12 mmol) were dissolved in Dioxane (6 mL), cesium carbonate (39.0 mg, 0.12 mmol), Ruphos-Pd-G₃ (3.0 mg, 0.004 mmol) and Ruphos (2.0 mg, 0.004 mmol) were added, the reaction solution was heated to 100 degrees and stirred overnight. The reaction solution was filtered through celite, and the filtrate was concentrated under reduced pressure. The obtained crude product was separated by HPLC preparation to obtain the compound of Example 61 (light yellow solid, 3 mg). LC-MS[M+H]⁺: m/z 563.3. ¹H NMR (400 MHz, MeOD-d₄): δ 7.64-7.62 (m, 1H), 7.50-7.45 (m, 4H), 6.95 (s, 1H), 5.94-5.92 (m, 1H), 4.32-4.30 (m, 2H), 3.87-3.85 (i, 4H), 3.73 (s, 3H), 3.263.15 (mi, 4H), 3.03-2.98 (i, 4H), 2.48 (s, 3H), 1.85 (m, 4H), 1.76 (d, J=7.2 Hz, 3H).

Referring to the method of Example 61, Examples 62-67 were obtained by the synthetic method that 1-(2-bromobenzene) pyrrolidine was replaced with different aryl brominated substances as raw materials:

| No | structure | analysis data |
|---|---|---|
| 62 | 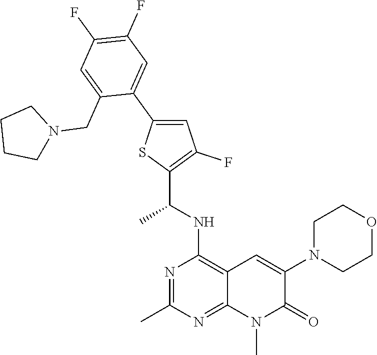 [SMILES: Cc1nc(N[C@H](C)c2sc(-c3cc(F)c(F)cc3CN3CCCC3)cc2F)c2cc(N3CCOCC3)c(=O)n(C)c2n1] | LC-MS[M + H]⁺: m/z 599.3. ¹H NMR (400 MHz, MeOD-d₄): δ 7.49 (m, 1H), 7.47-7.40 (m, 2H), 6.98 (s, 1H), 5.90-5.92 (m, 1H), 4.47 (s, 2H), 3.87-3.85 (m, 4H), 3.73 (s, 3H), 3.31-3.36 (m, 2H), 3.14-3.17 (m, 4H), 2.98-3.02 (m, 2H), 2.49 (s, 3H), 1.91-1.89 (m, 4H), 1.77 (d, J = 6.8 Hz, 3H). |
| 63 | 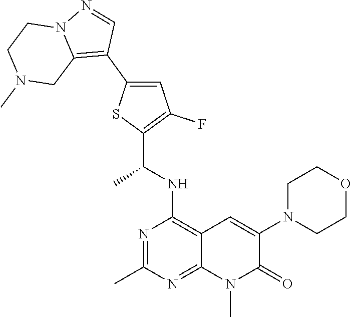 [SMILES: Cc1nc(N[C@H](C)c2sc(-c3cnn4c3CN(C)CC4)cc2F)c2cc(N3CCOCC3)c(=O)n(C)c2n1] | LC-MS [M + H]⁺: m/z 539.3. ¹H NMR (400 MHz, MeOD-d₄): δ 8.64 (s, 1H), 7.45 (m, 1H), 6.95 (s, 1H), 5.94-5.92 (m, 1H), 3.87- 3.85 (m, 4H), 3.23 (s, 2H), 3.17 (m, 2H), 3.03-2.98 (m, 4H), 2.48 (s, 3H), 2.38 (m, 2H), 2.35 (s, 3H), 1.76 (d, J = 7.2 Hz, 3H). |
| 64 | 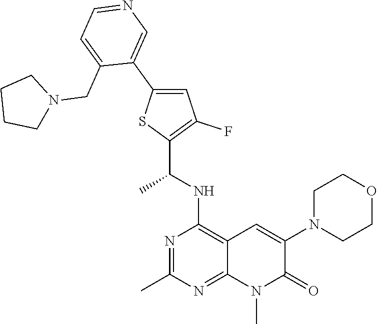 [SMILES: Cc1nc(N[C@H](C)c2sc(-c3cnccc3CN3CCCC3)cc2F)c2cc(N3CCOCC3)c(=O)n(C)c2n1] | LC-MS [M + H]⁺: m/z 564.3. ¹H NMR (400 MHz, MeOD-d₄): δ 8.35 (m, 1H), 7.94 (m, 1H), 7.69-7.47 (m, 3H), 6.94 (s, 1H), 5.93- 5.91 (m, 1H), 4.47 (s, 2H), 3.87-3.85 (m, 4H), 3.73 (s, 3H), 3.36- 3.31 (m, 2H), 3.17-3.14 (m, 4H), 3.05-2.92 (m, 2H), 2.49 (s, 3H), 1.91-1.89 (m, 4H), 1.77 (d, J = 6.8 Hz, 3H). |
| 65 | 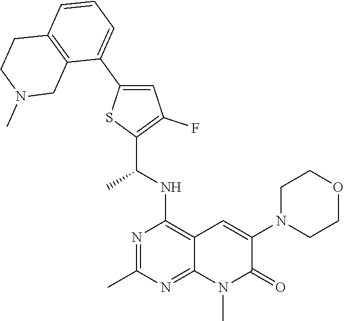 [SMILES: Cc1nc(N[C@H](C)c2sc(-c3cccc4c3CN(C)CC4)cc2F)c2cc(N3CCOCC3)c(=O)n(C)c2n1] | LC-MS [M + H]⁺: m/z 549.3. ¹H NMR (400 MHz, DMSO-d₆): δ 8.34 (d, J = 8.4 Hz, 1H), 8.10 (s, 1H), 7.43-7.39 (m, 1H), 7.37- 7.34 (m, 2H), 7.18 (m, 1H), 7.07 (s, 1H), 5.93-5.89 (m, 1H), 3.88- 3.85 (m, 4H), 3.58 (s, 3H), 3.27-3.24 (m, 4H), 3.29 (s, 2H), 2.95- 2.75 (m, 3H), 2.44 (s, 3H), 2.35-2.32 (m, 2H), 2.28 (s, 3H), 1.67 (d, J = 6.8 Hz, 3H). |
| 66 | 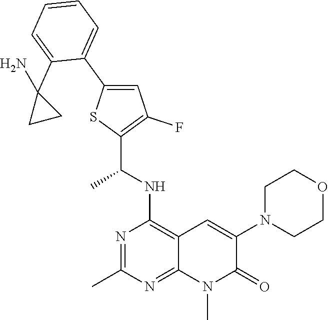 [SMILES: Cc1nc(N[C@H](C)c2sc(-c3ccccc3C3(N)CC3)cc2F)c2cc(N3CCOCC3)c(=O)n(C)c2n1] | LC-MS [M + H]⁺: m/z 535.3. ¹H NMR (400 MHz, DMSO-d₆): δ 8.35 (m, 1H), 8.09 (s, 1H), 7.43-7.34 (m, 3H), 7.18 (m, 1H), 7.07 (s, 1H), 5.93-5.83 (m, 1H), 3.88-3.85 (m, 4H), 3.58 (s, 3H), 3.27- 3.24 (m, 4H), 3.01-3.05 (m, 1H), 2.44 (s, 3H), 1.67 (d, J = 6.8 Hz, 3H), 0.7 (m, 2H), 0.5 (m, 2H). |
| 67 | 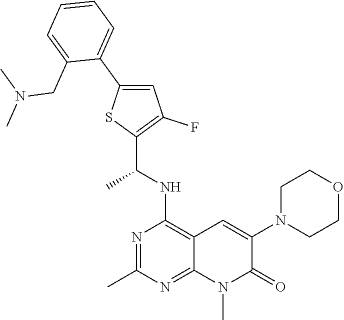 [SMILES: Cc1nc(N[C@H](C)c2sc(-c3ccccc3CN(C)C)cc2F)c2cc(N3CCOCC3)c(=O)n(C)c2n1] | LC-MS [M + H]⁺: m/z 537.2. ¹H NMR (400 MHz, DMSO-d₆): δ 8.33 (d, J = 8.4 Hz, 1H), 8.09 (s, 1H), 7.43-7.36 (m, 3H), 7.18 (m, 1H), 7.07 (s, 1H), 5.89-5.93 (m, 1H), 3.88-3.85 (m, 4H), 3.58 (s, 3H), 3.27-3.24 (m, 4H), 3.29 (s, 2H), 3.01-3.05 (m, 1H), 2.44 (s, 3H), 2.10 (s, 6H), 1.67 (d, J = 6.8 Hz, 3H). |
| 68 | 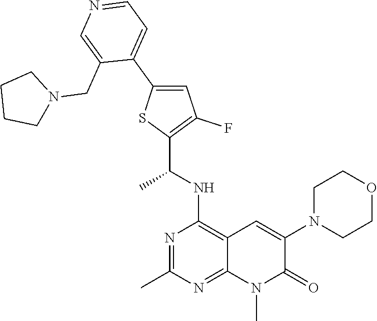 [SMILES: Cc1nc(N[C@H](C)c2sc(-c3ccncc3CN3CCCC3)cc2F)c2cc(N3CCOCC3)c(=O)n(C)c2n1] | LC-MS [M + H]⁺: m/z 564.3. ¹H NMR (400 MHz, MeOD-d₄): δ 8.25-8.13 (m, 2H), 7.49-7.40 (m, 2H), 6.95 (s, 1H), 5.94-5.91 (m, 1H), 4.47 (s, 2H), 3.87-3.85 (m, 4H), 3.73 (s, 3H), 3.36-3.31 (m, 2H), 3.17-3.14 (m, 4H), 3.02-2.96 (m, 2H), 2.49 (s, 3H), 1.91- 1.85 (m, 4H), 1.77 (d, J = 6.8 Hz, 3H). |

### Example 69: (R)-N-(1-(3-amino-5-(trifluoromethyl) phenyl) ethyl-2-methyl-6-morpholinimidazole [1′,2′:1,6] pyrido [2,3-d] pyrimidin-4-amine

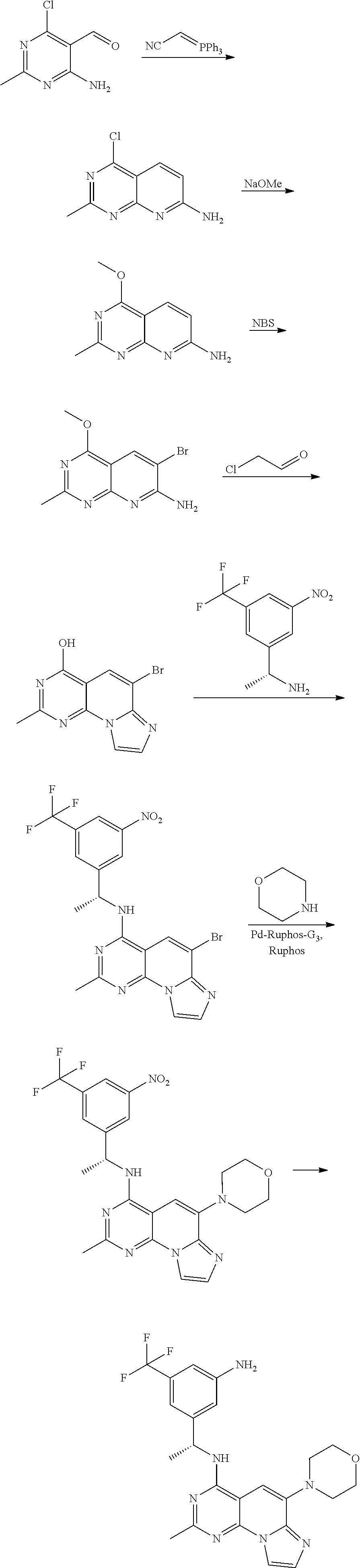

Step 1: At room temperature, 4-amino-6-chloro-2-methylpyrimidine-5-carbaldehyde (2 g, 11.7 mmol) was dissolved in acetonitrile (30 mL) solution, (triphenylphosphine) acetonitrile (3.5 g, 11.7 mmol) was added, and the reaction mixture was reacted at 85 degrees for 6 hours. The solvent of the reaction solution was removed by concentration under reduced pressure to obtain a crude intermediate product (2.0 g). LC-MS[M+H]⁺: m/z 195.2.

Step 2: The above intermediate compound (2.0 g, 10.0 mmol) was dissolved in MeOH (30 mL), sodium methoxide (1.6 g, 30.0 mmol) was added, and the reaction mixture was heated to 85° C. and reacted for 48 hours. LCMS detection showed that the reaction was completed. The mixture was concentrated under reduced pressure to remove most of the mixture, ethyl acetate (50 mL) was added to the concentrated residue, and the mixture was washed with water (50 mL). The separated organic phase was concentrated under reduced pressure to give the compound as a yellow solid (500 mg). LC-MS[M+H]⁺: m/z 191.3.

Step 3: the above intermediate compound (100 mg, 0.5 mmol) was dissolved in N,N-dimethylformamide (10 mL), bromosuccinimide (93.1 mg, 0.5 mmol) was added, and the mixture was reacted at room temperature for one hour. The reaction solution was concentrated under reduced pressure, and the obtained crude product was purified by silica gel column chromatography (eluent: petroleum ether/ethyl acetate 2:1) to give the intermediate compound as a white solid (60 mg). LC-MS[M+H]⁺: m/z 271.0 Step 4: Under nitrogen protection, the above intermediate compound (200 mg, 0.74 mmol) was dissolved in chloroacetaldehyde (5 mL), and the mixture was heated to 85° C. to react overnight. The reaction mixture was concentrated under reduced pressure and purified by HPLC preparation to give a white product intermediate compound (61 mg). LC-MS [M+H]⁺: m/z 279.1.

Step 5: Under nitrogen protection, the above intermediate compound (200 mg, 0.75 mmol), (R)-1-(3-nitro-5-(trifluoromethyl) phenyl) ethyl-1-amine (210.6 mg, 0.90 mmol), BOP (499.2 mg, 1.13 mmol) and DBU (256.1 mg, 1.13 mmol) were dissolved in N,N-dimethylformamide (10 mL). The reaction solution was stirred for 18 hours at room temperature. The reaction was concentrated under reduced pressure, the crude product was purified by silica gel column chromatography (eluent: petroleum ether/ethyl acetate 3:1) to give the intermediate product as a yellow solid (91 mg). LC-MS[M+H]⁺: m/z 497.2.

Step 6: Under nitrogen protection, the above intermediate compound (50 mg, 0.10 mmol), morpholine (26 mg, 0.30 mmol), RuPhos (4 mg, 0.001 mmol), Pd-Ruphos-G₃ (8 mg, 0.001 mmol) and cesium carbonate (97.8 mg, 0.30 mmol) were dissolved in toluene (10 mL), and the reaction solution was heated to 110° C. to react for 18 hours. The reaction solution was concentrated under reduced pressure, and the obtained crude product was purified by silica gel column chromatography (eluent: dichloromethane/methanol 30:1) to give the intermediate product as a white solid (5.0 mg). LC-MS[M+H]⁺: m/z 502.3.

Step 7: The above intermediate compound (10 mg, 0.02 mmol) was dissolved in tert-butanol (5 mL), Pd/C (1 mg) was added, and the reaction mixture was stirred at room temperature overnight under hydrogen atmosphere (1 atm). The reaction solution was filtered, and the filtrate was concentrated under reduced pressure. The resulting crude product was purified by HPLC preparation to give the compound of Example 69 as a white solid (1.1 mg). LC-MS[M+H]⁺: m/z 472.1. ¹H NMR (400 MHz, MeOD-d₄): δ 8.37 (s, 1H), 7.55 (s, 1H), 7.28 (s, 1H), 6.97-7.03 (m, 2H), 6.80-6.85 (m, 1H), 5.61-5.63 (m, 1H), 3.95-3.97 (m, 4H), 3.40-3.48 (i, 4H) 2.53 (s, 3H), 1.63-1.66 (m, 3H).

Referring to the method of Example 69, Examples 70-77 were obtained by the synthetic method that 1-(2-bromobenzene) pyrrolidine was replaced with different aryl brominated substances as raw materials:

| | | |
|---|---|---|
| 70 | 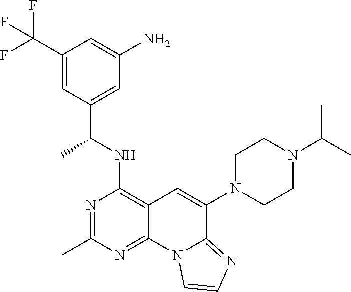 [SMILES: Cc1nc(N[C@H](C)c2cc(N)cc(C(F)(F)F)c2)c2cc(N3CCN(C(C)C)CC3)c3nccn3c2n1] | LC-MS [M + H]⁺: m/z 513.6. ¹H NMR (400 MHz, MeOD-d₄): δ 8.52 (s, 1H), 7.78 (m, 1H), 7.73 (s, 1H), 7.05 (s, 2H), 6.85 (s, 1H), 5.65-5.63 (m, 1H), 4.05 (m, 2H), 3.81-3.79 (m, 2H), 3.78 (m, 2H), 3.47 (m, 2H), 3.26 (m, 1H), 2.56 (s, 3H), 1.83-1.78 (d, J = 6.4 Hz, 6H), 1.68 (d, J = 6.8 Hz, 3H). |
| 71 | 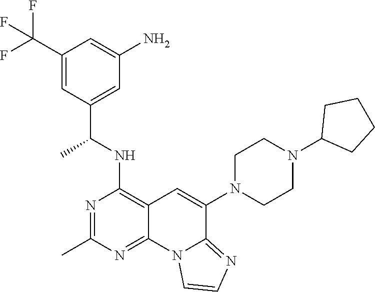 [SMILES: Cc1nc(N[C@H](C)c2cc(N)cc(C(F)(F)F)c2)c2cc(N3CCN(C4CCCC4)CC3)c3nccn3c2n1] | LC-MS [M + H]⁺: m/z 539.2. ¹H NMR (400 MHz, MeOD-d₄): δ 8.52 (s, 1H), 7.78 (m, 1H), 7.73 (s, 1H), 7.05 (s, 2H), 6.85 (s, 1H), 5.65-5.63 (m, 1H), 4.05 (m, 2H), 3.81-3.79 (m, 2H), 3.78 (m, 2H), 3.47 (m, 2H), 3.26 (m, 1H), 2.56 (s, 3H), 1.89-1.75 (m, 8H), 1.60 (d, J = 6.8 Hz, 3H). |
| 72 | 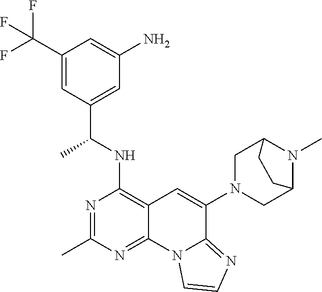 [SMILES: Cc1nc(N[C@H](C)c2cc(N)cc(C(F)(F)F)c2)c2cc(N3CC4CCC(C3)N4C)c3nccn3c2n1] | LC-MS [M + H]⁺: m/z 511.1. ¹H NMR (400 MHz, MeOD-d₄): δ 8.27 (s, 1H), 7.46 (s, 1H),7.05 (s, 1H), 6.89 (m, 2H), 6.71 (s, 1H), 5.54-5.52 (m, 1H), 4.27-4.19 (m, 2H), 3.95-3.86 (m, 2H), 3.16-3.13 (m, 2H), 2.70 (s, 3H), 2.43 (s, 3H), 2.34-2.31 (m, 2H), 2.11-2.07 (m, 2H), 1.94-1.92 (m, 1H), 1.89-1.87 (m, 2H), 1.78- 1.65 (m, 6H), 1.55 (d, J = 7.2 Hz, 3H). |
| 73 | 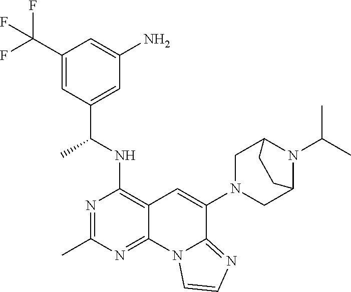 [SMILES: Cc1nc(N[C@H](C)c2cc(N)cc(C(F)(F)F)c2)c2cc(N3CC4CCC(C3)N4C(C)C)c3nccn3c2n1] | LC-MS [M + H]⁺: m/z 539.2. ¹H NMR (400 MHz, MeOD-d₄): δ 8.35 (s, 1H), 7.53 (s, 1H), 7.05 (s, 1H), 6.99-6.97 (m, 2H), 6.79 (s, 1H), 5.61-5.63 (m, 1H), 4.12-4.08 (m, 2H), 3.78-3.76 (m, 2H), 3.29-3.13 (m, 3H), 2.51 (s, 3H), 2.20-2.16 (m, 2H), 2.05-2.00 (m, 2H), 1.72 (d, J = 7.2 Hz,3H), 1.31-1.25 (m, 6H). |
| 74 | 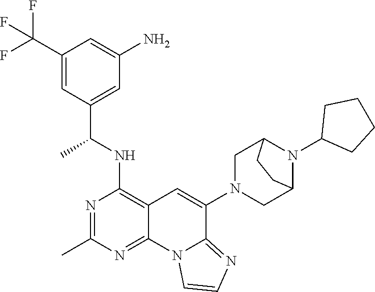 [SMILES: Cc1nc(N[C@H](C)c2cc(N)cc(C(F)(F)F)c2)c2cc(N3CC4CCC(C3)N4C3CCCC3)c3nccn3c2n1] | LC-MS [M + H]⁺: m/z 565.2. ¹H NMR (400 MHz, MeOD-d₄): δ 8.35 (s, 1H), 7.52 (s, 1H), 7.05 (s, 1H), 6.98 (m, 2H), 6.79 (s, 1H), 5.64-5.62 (m, 1H), 4.27-4.18 (m, 2H), 3.75-3.67 (m, 2H), 3.15-3.13 (m, 3H), 2.51 (s, 3H), 2.34-2.11 (m, 8H), 1.64-1.62 (m, 2H), 1.65 (d, J = 7.2 Hz, 3H), 1.33-1.29 (m, 2H). |

### Example 75: (R)-N-(1-(3-amino-5-(trifluoromethyl) phenyl) ethyl-6-(4-methoxytetrahydro-2H-pyran-4-yl) 2-methylimidazole [1′,2′:1,6] pyrido [2,3-d] pyrimidin-4-amine

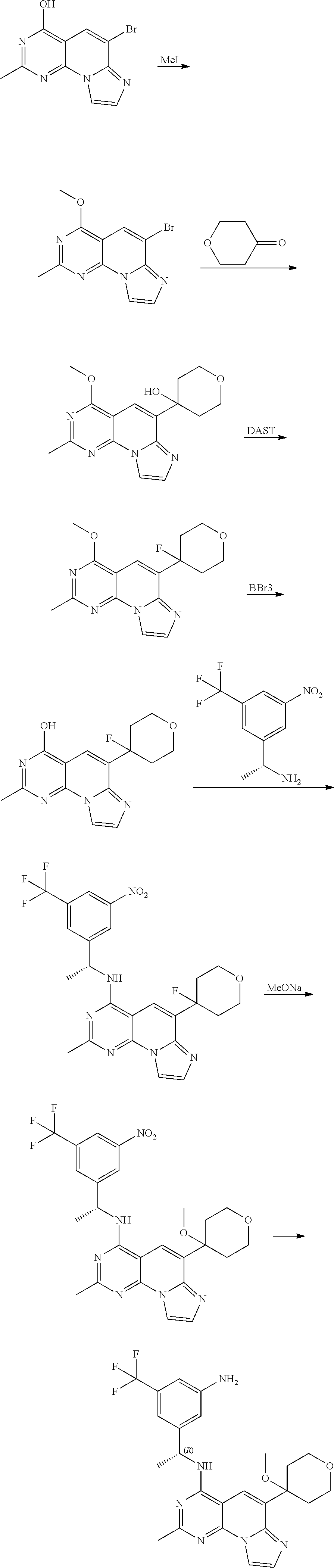

Step 1: under nitrogen protection, methyl iodide (2 g, 14.4 mmol) and potassium carbonate (3.5 g, 25.3 mmol) were added to a solution of 6-bromo-2-methylimidazole [1′,2′:1,6] pyrido [2,3-d] pyrimidin-4-ol (2 g, 7.2 mmol) in tetrahydrofuran (50 mL), the reaction solution was heated to 60 degrees and stirred to react overnight. The reaction solution was concentrated under reduced pressure to remove the solvent, and the obtained crude product was purified by silica gel column chromatography (eluent: petroleum ether/ethyl acetate=4:1) to give the intermediate compound as a yellow solid (1.5 g, 5.1 mmol). LC-MS[M+H]⁺: m/z 293.0/295.0.

Step 2: Under nitrogen protection, a solution of the above intermediate compound (500 mg, 1.7 mmol) in tetrahydrofuran (30 mL) was cooled to −20 degrees, and then isopropyl magnesium chloride (1M in THF, 2 mL, 2 mmol) was slowly added dropwise to the solution. The reaction solution was slowly warmed to room temperature and stirred for 30 minutes. Then, a solution of tetrahydropyran-4-one (200 mg, 2 mmol) in tetrahydrofuran (2 mL) was slowly added to the above reaction solution under ice-cooling. After stirring at zero degrees for 30 minutes, the mixture was slowly warmed to room temperature, and continued to stir for 2 hours. After LC-MS detection showed that the reaction was almost completed, water (50 mL) was added to the reaction mixture, and the mixture was extracted twice with ethyl acetate (50 mL). The combined organic phases were dried over anhydrous sodium sulfate, and concentrated under reduced pressure. The resulting crude product was purified by silica gel column chromatography (eluent: petroleum ether/ethyl acetate=3:1) to give a white solid compound (287 mg). LC-MS[M+H]⁺: m/z 315.4.

Step 3: Under nitrogen protection, diethylamine sulfur trifluoride (200 mg, 1.2 mmol) was added to a solution of the above intermediate compound (286 mg, 0.9 mmol) in dichloromethane (10 mL). The reaction solution was stirred at room temperature for 2 hours. LC-MS detection showed that the reaction was almost completed, saturated aqueous sodium bicarbonate (10 mL) was added to the reaction mixture. The separated organic phase was dried over anhydrous sodium sulfate, and concentrated under reduced pressure. The obtained crude product was purified by silica gel column chromatography (eluent: petroleum ether/ethyl acetate=4:1) to give a white solid compound (130 mg). LC-MS[M+H]⁺: m/z 317.3.

Step 4: Under nitrogen protection, a solution of the above intermediate compound (130 mg, 0.4 mmol) in dichloromethane (10 mL) was cooled to zero degree, and then BBr₃ (150 mg, 0.6 mmol) was added. The reaction solution was stirred at room temperature for 30 minutes. The reaction solution was concentrated under reduced pressure to give a brown solid crude product (73 mg). LC-MS[M+H]⁺: m/z 303.2.

Step 5: Under nitrogen protection, the above intermediate compound (70 mg, 0.23 mmol), (R)-1-(3-nitro-5-(trifluoromethyl) phenyl) ethyl-1-amine (56 mg, 0.24 mmol), BOP (133 mg, 0.3 mmol) and DBU (60 mg, 0.4 mmol) were dissolved in N,N-dimethylformamide (10 mL). The reaction solution was stirred for 18 hours at room temperature. The reaction solution was concentrated under reduced pressure, the crude product was purified by silica gel column chromatography (eluent: petroleum ether/ethyl acetate 3:1) to give a white solid product (55 mg). LC-MS[M+H]⁺: m/z 519.5.

Step 6: To a solution of the above intermediate (52 mg, 0.1 mmol) in methanol (10 mL) was added sodium methoxide (162 mg, 0.3 mmol). The reaction solution was heated to reflux for 6 hours. LC-MS detection showed that the reaction of raw materials was completed. Water (10 mL) was added to the reaction solution, the pH of the solution was adjusted to 8 with 1N dilute hydrochloric acid, and the reaction solution was extracted with ethyl acetate (30 mL). The separated organic phase was concentrated under reduced pressure, and the obtained crude product was purified by silica gel column chromatography (eluent: petroleum ether/ethyl acetate 3:1) to give a white solid product (22 mg). LC-MS[M+H]⁺: m/z 531.5.

Step 7: the above intermediate (22 mg, 0.04 mmol) was dissolved in tert-butanol (5 mL), Pd/C (1 mg) was added, and the reaction solution was stirred at room temperature overnight under hydrogen atmosphere (1 atm). The reaction solution was filtered, and the filtrate was concentrated under reduced pressure. The resulting crude product was purified by HPLC preparation to give the compound of Example 75 as a white solid (7 mg). LC-MS[M+H]⁺: m/z 501.5. ¹H NMR (400 MHz, CD₃OD): δ 8.40 (s, 1H), 8.05 (s, 1H), 7.62 (s, 1H), 7.02-7.00 (m, 2H), 6.80 (s, 1H), 5.63-5.60 (1N, 1H), 4.16-4.13 (i, 2H), 3.72-3.69 (m, 2H), 3.57 (s, 3H), 2.58 (s, 3H), 2.51-2.49 (i, 2H), 2.02-1.99 (m, 2H), 1.68 (d, J=7.2 Hz, 3H).

Referring to the method of Example 75, Examples 76-81 were obtained by the synthesis method that tetrahydropyran-4-one was replaced with different ketones as raw materials and (R)-1-(3-nitro-5-(trifluoromethyl) phenyl) ethyl-1-amine was replaced with different benzylamine as raw materials:

| | | |
|---|---|---|
| 76 | 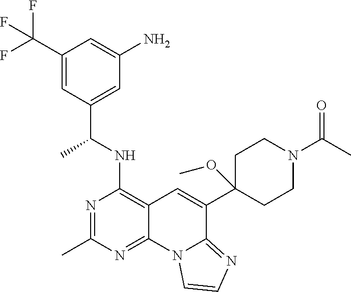 [SMILES: COC1(c2cc3c(N[C@H](C)c4cc(N)cc(C(F)(F)F)c4)nc(C)nc3n3ccnc23)CCN(C(C)=O)CC1] | LC-MS[M + H]⁺: m/z 542.3. ¹H NMR (400 MHz, CD₃OD): δ 8.38 (s, 1H), 8.03 (s, 1H), 7.61 (s, 1H), 7.02-7.00 (m, 2H), 6.82 (s, 1H), 5.66-5.64 (m, 1H), 4.59-4.56 (m, 1H), 3.90-3.89 (m, 1H), 3.77-3.74 (m, 1H), 3.55 (s, 3H), 3.25-3.23 (m, 1H), 2.57 (s, 3H), 2.55-2.52 (m, 2H), 2.20 (s, 3H), 2.07-1.96 (m, 2H), 1.68 (d, J = 7.2 Hz, 3H). |
| 77 | 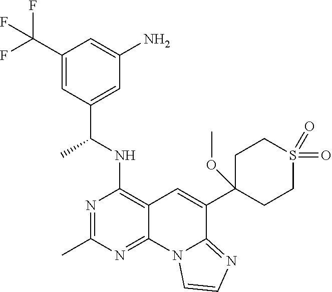 [SMILES: COC1(c2cc3c(N[C@H](C)c4cc(N)cc(C(F)(F)F)c4)nc(C)nc3n3ccnc23)CCS(=O)(=O)CC1] | LC-MS[M + H]⁺: m/z 549.6. ¹H NMR (400 MHz, CD₃OD): δ 8.35 (s, 1H), 8.04 (s, 1H), 7.62 (s, 1H), 7.03 (m, 2H), 6.83 (s, 1H), 5.66-5.64 (m, 1H), 4.59-4.56 (m, 1H), 3.90-3.89 (m, 1H), 3.77- 3.75 (m, 1H), 3.57 (s, 3H), 3.25-3.23 (m, 1H), 2.65-2.53 (m, 5H), 2.07-1.95 (m, 2H), 1.70 (d, J = 7.2 Hz, 3H). |
| 78 | 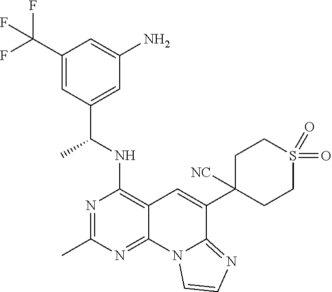 [SMILES: Cc1nc(N[C@H](C)c2cc(N)cc(C(F)(F)F)c2)c2cc(C3(C#N)CCS(=O)(=O)CC3)c3nccn3c2n1] | LC-MS [M + H]⁺: m/z 544.6. ¹H NMR (400 MHz, CD₃OD): δ 8.36 (s, 1H), 8.03 (s, 1H), 7.61 (s, 1H), 7.03 (m, 2H), 6.82 (s, 1H), 5.66-5.63 (m, 1H), 4.59-4.56 (m, 1H), 3.90-3.89 (m, 1H), 3.77- 3.72 (m, 1H), 3.25-3.23 (m, 1H), 2.58 (s, 3H), 2.55-2.51 (m, 2H), 2.03-1.97 (m, 2H), 1.68 (d, J = 7.2 Hz, 3H). |
| 79 | 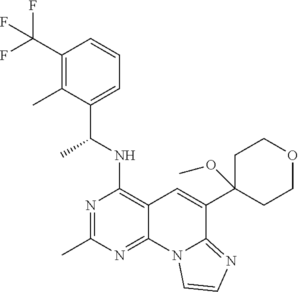 [SMILES: COC1(c2cc3c(N[C@H](C)c4cccc(C(F)(F)F)c4C)nc(C)nc3n3ccnc23)CCOCC1] | LC-MS [M + H]⁺: m/z 500.2. LC-MS [M + H]⁺: m/z 501.5. ¹H NMR (400 MHz, CD3OD): δ 7.95-7.68 (m, 3H), 7.02-7.00 (m, 2H), 6.85 (s, 1H), 5.63-5.60 (m, 1H), 4.16-4.13 (m, 2H), 3.72-3.69 (m, 2H), 3.57 (s, 3H), 2.58 (s, 3H), 2.51-2.49 (m, 2H), 2.28 (s, 3H), 2.05-1.97 (m, 2H), 1.68 (d, J = 7.2 Hz, 3H). |
| 80 | 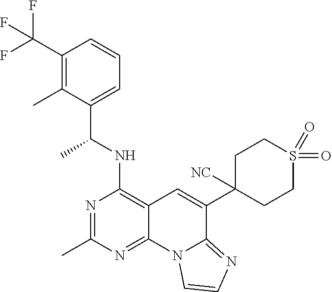 [SMILES: Cc1nc(N[C@H](C)c2cccc(C(F)(F)F)c2C)c2cc(C3(C#N)CCS(=O)(=O)CC3)c3nccn3c2n1] | LC-MS [M + H]⁺: m/z 543.6. ¹H NMR (400 MHz, CD₃OD): δ 7.93- 7.68 (m, 3H), 7.04-7.00 (m, 2H), 6.87 (s, 1H), 5.63-5.61 (m, 1H), 4.58-4.56 (m, 1H), 3.88-3.79 (m, 2H), 3.24-3.23 (m, 1H), 2.58 (s, 3H), 2.55-2.51 (m, 2H), 2.25 (s, 3H), 2.03-1.97 (m, 2H), 1.70 (d, J = 7.2 Hz, 3H). |
| 81 | 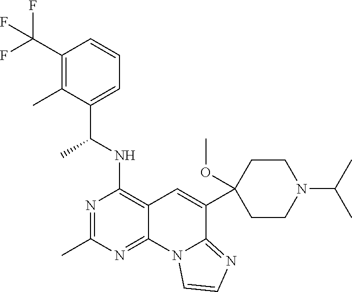 [SMILES: COC1(c2cc3c(N[C@H](C)c4cccc(C(F)(F)F)c4C)nc(C)nc3n3ccnc23)CCN(C(C)C)CC1] | LC-MS[M + H]⁺: m/z 541. ¹H NMR (400 MHz, CD₃OD): δ 7.75- 7.63 (m, 3H), 7.02-6.95 (m, 2H), 6.85 (s, 1H), 5.62-5.60 (m, 1H), 3.57 (s, 3H), 2.82-2.69 (m, 4H), 2.58 (s, 3H), 2.49-2.35 (m, 3H), 2.28 (s, 3H), 2.15-2.08 (m, 2H), 1.68 (d, J = 7.2 Hz, 3H), 1.40 (d, J = 6.8 Hz, 6H). |

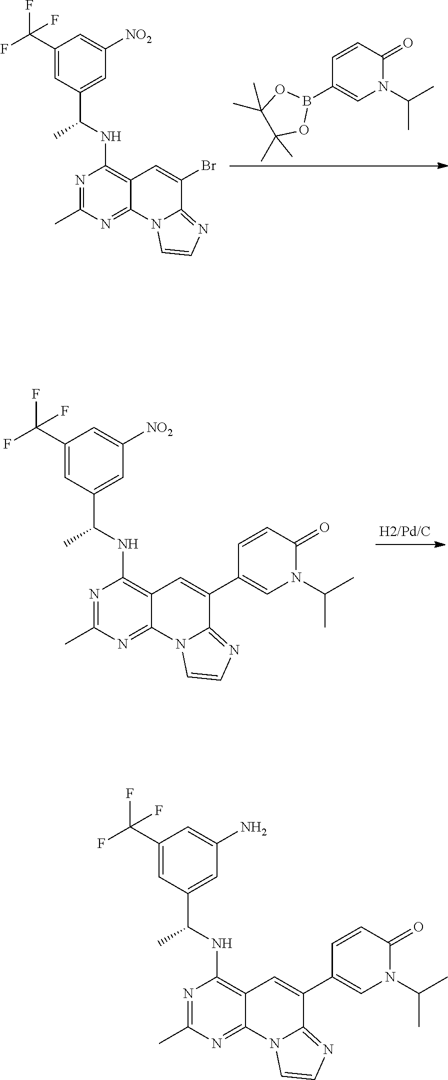 [SMILES: Cc1nc(N[C@H](C)c2cc([N+](=O)[O-])cc(C(F)(F)F)c2)c2cc(Br)c3nccn3c2n1 ; CC(C)n1cc(B2OC(C)(C)C(C)(C)O2)ccc1=O ; Cc1nc(N[C@H](C)c2cc([N+](=O)[O-])cc(C(F)(F)F)c2)c2cc(-c3ccc(=O)n(C(C)C)c3)c3nccn3c2n1 ; Cc1nc(N[C@H](C)c2cc(N)cc(C(F)(F)F)c2)c2cc(-c3ccc(=O)n(C(C)C)c3)c3nccn3c2n1]

Step 1: (R)-6-bromo-2-methyl-N-(1-(3-nitro-5-(trifluoromethyl) phenyl) ethyl) imidazo [1′,2′:1,6] pyrido [2,3-d] pyrimidin-4-amine (50.0 mg, 0.10 mmol) and 1-isopropyl-5-(4,4,5,5-tetramethyl-1,3,2-dioxaborolan-2-yl) pyridin-2(1H)-one (31 mg, 0.12 mmol) were dissolved in a mixed solvent of 1,4-dioxane and water (20 mL/4 mL). Potassium phosphate (64.0 mg, 0.3 mmol) and Pd(dppf)Cl₂ (5.0 mg, 0.01 mmol) were added to the above reaction solution under nitrogen protection, and the reaction solution was heated to 85° C. overnight. The reaction solution was filtered through celite and washed with ethyl acetate. The combined organic phase was concentrated under reduced pressure, and the obtained crude product was purified by silica gel column chromatography (eluent: petroleum ether/ethyl acetate=2/1) to give a white solid compound (30 mg). LC-MS[M+H]⁺: m/z 552.5.

Step 2: The above intermediate (30 mg, 0.05 mmol) was dissolved in tert-butanol (5 mL), Pd/C (2 mg) was added, and the reaction mixture was stirred at room temperature overnight under hydrogen atmosphere (1 atm). The reaction solution was filtered, and the filtrate was concentrated under reduced pressure. The resulting crude product was purified by HPLC preparation to give the compound of Example 82 as a white solid (10 mg). LC-MS[M+H]⁺: m/z 521.5. ¹H NMR (400 MHz, DMSO): δ 8.88 (s, 1H), 8.43 (s, 1H), 8.16-8.14 (m, 2H), 7.65 (s, 1H), 7.01-6.99 (m, 2H), 6.81 (s, 1H), 6.71-6.69 (m, 1H), 5.64-5.62 (m, 1H), 5.22 (m, 1H), 2.57 (s, 3H), 1.67-1.65 (d, J=7.2 Hz, 3H), 1.51-1.49 (d, J=6.8 Hz, 6H).

Referring to the method of Example 82, Examples 83-84 were obtained by the synthesis method that 1-isopropyl-5-(4,4,5,5-tetramethyl-1,3,2-dioxaborolan-2-yl)pyridin-2(1H)-one was replaced with different boronic acid starting materials:

| | | |
|---|---|---|
| 83 | 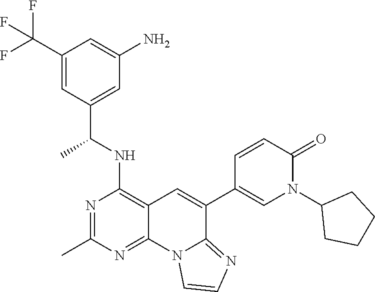 [SMILES: Cc1nc(N[C@H](C)c2cc(N)cc(C(F)(F)F)c2)c2cc(-c3ccc(=O)n(C4CCCC4)c3)c3nccn3c2n1] | LC-MS[M + H]+: m/z 548.3. 1H NMR (400 MHz, MeOD-d₄): δ 9.01 (s, 1H), 8.46 (s, 1H), 8.20-8.17 (m, 2H), 7.67 (s, 1H) , 7.02 (d, J = 8.0 Hz, 2H), 6.83 (s, 1H), 6.71 (m, 1H), 5.68-5.66 (m, 1H), 5.39-5.29 (m, 1H), 2.59 (s, 3H), 2.03-2.01 (m, 2H), 1.98-1.90 (m, 5H), 1.84-1.80 (m, 2H), 1.70 (d, J = 6.8 Hz, 3H). |
| 84 | 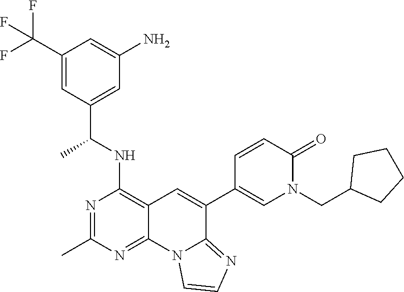 [SMILES: Cc1nc(N[C@H](C)c2cc(N)cc(C(F)(F)F)c2)c2cc(-c3ccc(=O)n(CC4CCCC4)c3)c3nccn3c2n1] | LC-MS[M + H]⁺: m/z 562.3. ¹H NMR (400 MHz, MeOD-d₄): δ 8.74 (s, 1H), 8.43 (s, 1H), 8.20-8.17 (m, 1H), 8.14 (s, 1H), 7.64 (s, 1H) , 7.00 (d, J = 8.0 Hz, 2H), 6.81 (s, 1H) , 6.69 (m, 1H), 5.65- 5.63 (m, 1H), 4.09-4.07 (m, 2H), 2.56 (s, 3H), 2.48-2.47 (m, 1H), 1.74-1.73 (m, 5H), 1.65 (d, J = 7.2 Hz, 3H), 1.60 (m, 2H), 1.38 (m, 2H). |

### Example 85: (R)-1-(4-(4-((1-(3-amino-5-(trifluoromethyl) phenyl) ethyl) amino)-2-methylimidazole [1′,2′:1,6] pyrido [2,3-d] pyrimidin-6-yl)-4-hydroxypiperidin-1-yl) ethyl-1-one

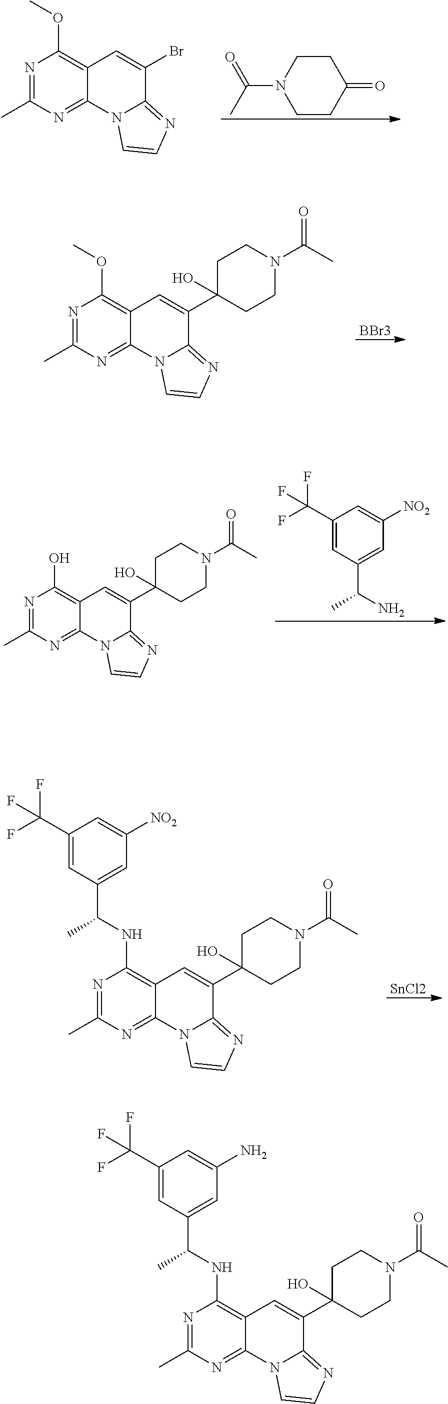

Step 1: Under nitrogen protection, a solution of 6-bromo-4-methoxy-2 methylimidazole [1′,2′:1,6] pyrido [2,3-d] pyrimidine (150 mg, 0.52 mmol) in tetrahydrofuran (10 mL) was cooled to −78 degrees, and then n-butyl lithium (1.6M in THF, 1.3 mL, 2.08 mmol) was slowly added dropwise to the solution. The reaction solution was slowly warmed to room temperature and stirred for 60 minutes. Then, under ice-bath-cooling, a solution of acetylpiperidin-4-one (147 mg, 1.04 mmol) in tetrahydrofuran (2 mL) was slowly added to the above reaction solution. After stirring at zero degrees for 30 minutes, the mixture was slowly warmed to room temperature, and continued to be stirred for 2 hours. After LC-MS detection showed that the reaction was almost completed, water (20 mL) was added to the reaction mixture, and the mixture was extracted twice with ethyl acetate (20 mL). The combined organic phases were dried over anhydrous sodium sulfate, and concentrated under reduced pressure. The resulting crude product was purified by silica gel column chromatography (eluent: petroleum ether/ethyl acetate=3:1) to give a white solid compound (30 mg). LC-MS[M+H]⁺: m/z 356.2.

Step 2: Under nitrogen protection, a solution of the above intermediate compound (30 mg, 0.08 mmol) in dichloromethane (5 mL) was cooled to zero degree, and then BBr3 (1M in DCM, 0.8 mL, 0.8 mmol) was added. The reaction solution was stirred at room temperature overnight. The reaction solution was concentrated under reduced pressure to give a brown solid crude product (20 mg). LC-MS[M+H]⁺: m/z 342.2.

Step 3: Under nitrogen protection, the above intermediate compound (20 mg, 0.06 mmol), (R)-1-(3-nitro-5-(trifluoromethyl) phenyl) ethyl-1-amine (16 mg, 0.07 mmol), BOP (44 mg, 0.1 mmol) and DBU (22 mg, 0.1 mmol) were dissolved in N,N-dimethylformamide (5 mL). The reaction solution was stirred for 12 hours at room temperature. The mixture was concentrated under reduced pressure, the crude product was purified by silica gel column chromatography (eluent: petroleum ether/ethyl acetate 3:1) to give a white solid product (15 mg). LC-MS[M+H]⁺: m/z 558.2.

Step 4: the above intermediate (15 mg, 0.03 mmol) was dissolved in a mixed solution of tetrahydrofuran/ethanol (1 mL/3 mL), and stannous chloride (28 mg, 0.15 mmol) was added. The reaction mixture was stirred overnight at room temperature. The reaction solution was filtered, and the filtrate was concentrated under reduced pressure. The resulting crude product was purified by HPLC preparation to give the compound of Example 85 as a white solid (2.1 mg). LC-MS[M+H]⁺: m/z 528.2. ¹H NMR (400 MHz, CD₃OD): δ 8.38 (s, 1H), 8.03 (s, 1H), 7.61 (s, 1H), 7.02-7.00 (m, 2H), 6.83 (s, 1H), 5.66-5.64 (m, 1H), 4.59-4.56 (m, 1H), 3.90-3.89 (m, 1H), 3.77-3.74 (m, 1H), 3.25-3.23 (m, 1H), 2.57 (s, 3H), 2.55-2.52 (m, 2H), 2.20 (s, 3H), 2.07-1.96 (m, 2H), 1.68 (d, J=7.2 Hz, 3H).

Referring to the methods of Examples 75 and 85, Examples 86-93 were obtained by the synthesis method that tetrahydropyran-4-one or acetylpiperidin-4-one was replaced with different ketones as starting materials and (R)-1-(3-nitro-5-(trifluoromethyl) phenyl) ethyl-1-amine was replaced with different benzylamines as starting materials:

| | | |
|---|---|---|
| 86 | 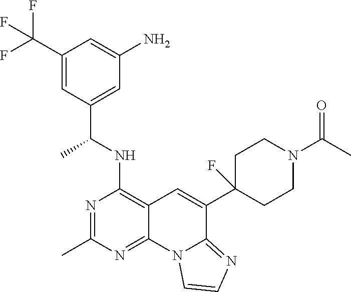 [SMILES: CC(=O)N1CCC(F)(c2cc3c(N[C@H](C)c4cc(N)cc(C(F)(F)F)c4)nc(C)nc3n3ccnc23)CC1] | LC-MS[M + H]⁺: m/z 530.2. ¹H NMR (400 MHz, CD₃OD): δ 8.38 (s, 1H), 8.03 (s, 1H), 7.61 (s, 1H), 7.02-7.00 (m, 2H), 6.83 (s, 1H), 5.66-5.64 (m, 1H), 3.25-3.23 (m, 2H), 2.57 (s, 3H), 2.55- 2.52 (m, 2H), 2.20 (s, 3H), 1.70 (d, J = 7.2 Hz, 3H), 1.54-1.27 (m, 4H). |
| 87 | 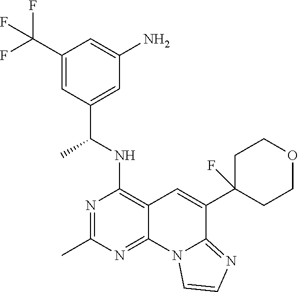 [SMILES: Cc1nc(N[C@H](C)c2cc(N)cc(C(F)(F)F)c2)c2cc(C3(F)CCOCC3)c3nccn3c2n1] | LC-MS[M + H]⁺: m/z 489.2. ¹H NMR (400 MHz, CD₃OD): δ 8.38 (s, 1H), 8.02 (s, 1H), 7.60 (s, 1H), 7.02-6.98 (m, 2H), 6.81 (s, 1H), 5.66-5.64 (m, 1H), 3.75-3.63 (m, 4H), 2.57 (s, 3H), 2.55- 2.52 (m, 2H), 1.70 (d, J = 7.2 Hz, 3H), 1.63-1.47 (m, 4H). |
| 88 | 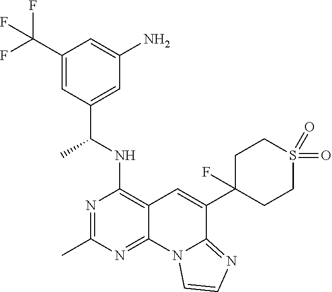 [SMILES: Cc1nc(N[C@H](C)c2cc(N)cc(C(F)(F)F)c2)c2cc(C3(F)CCS(=O)(=O)CC3)c3nccn3c2n1] | LC-MS[M + H]⁺: m/z 537.2. ¹H NMR (400 MHz, CD₃OD): δ 8.38 (s, 1H), 8.05 (s, 1H), 7.61 (s, 1H), 7.02-6.99 (m, 2H), 6.83 (s, 1H), 5.66-5.63 (m, 1H), 2.95-2.83 (m, 4H), 2.55 (s, 3H), 2.53- 2.51 (m, 2H), 1.68 (d, J = 7.2 Hz, 3H), 1.65-1.45 (m, 4H). |
| 89 | 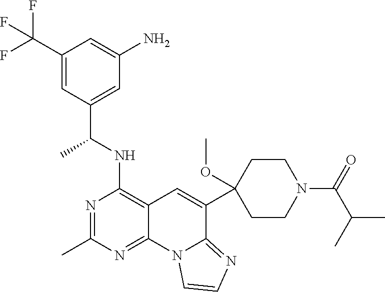 [SMILES: COC1(c2cc3c(N[C@H](C)c4cc(N)cc(C(F)(F)F)c4)nc(C)nc3n3ccnc23)CCN(C(=O)C(C)C)CC1] | LC-MS[M + H]⁺: m/z 570.2. ¹H NMR (400 MHz, CD₃OD): δ 8.38 (s, 1H), 8.03 (s, 1H), 7.61 (s, 1H), 7.02-7.00 (m, 2H), 6.83 (s, 1H), 5.66-5.64 (m, 1H), 4.59-4.56 (m, 1H), 3.90-3.89 (m, 1H), 3.77-3.74 (m, 1H), 3.58 (s, 3H), 3.25-3.23 (m, 1H), 2.57 (s, 3H), 2.55-2.52 (m, 2H), 2.23 (m, 1H), 2.07-1.96 (m, 2H), 1.68 (d, J = 7.2 Hz, 3H), 1.30 (d, J = 6.8 Hz, 6H). |
| 90 | 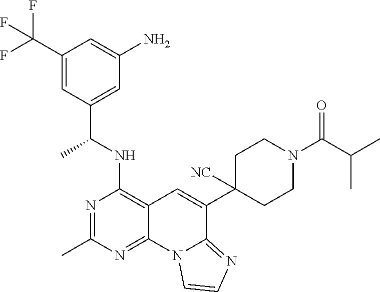 [SMILES: Cc1nc(N[C@H](C)c2cc(N)cc(C(F)(F)F)c2)c2cc(C3(C#N)CCN(C(=O)C(C)C)CC3)c3nccn3c2n1] | LC-MS[M + H]⁺: m/z 565.2. ¹H NMR (400 MHz, CD₃OD): δ 8.35 (s, 1H), 8.02 (s, 1H), 7.63 (s, 1H), 7.02-6.99 (m, 2H), 6.85 (s, 1H), 5.67-5.64 (m, 1H), 4.55-4.52 (m, 1H), 3.90-3.75 (m, 2H), 3.31-3.27 (m, 1H), 2.57 (s, 3H), 2.57-2.54 (m, 2H), 2.23 (m, 1H), 2.07-1.96 (m, 2H), 1.68 (d, J = 7.2 Hz, 3H), 1.38-1.36 (d, J = 6.8 Hz, 6H). |
| 91 | 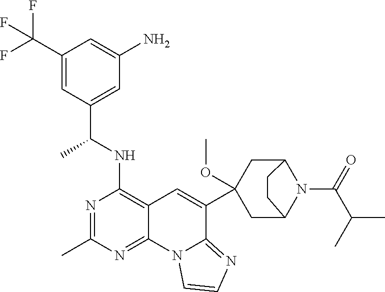 [SMILES: COC1(c2cc3c(N[C@H](C)c4cc(N)cc(C(F)(F)F)c4)nc(C)nc3n3ccnc23)CC2CCC(C1)N2C(=O)C(C)C] | LC-MS[M + H]⁺: m/z 596.2. ¹H NMR (400 MHz, CD₃OD): δ 8.38 (s, 1H), 8.03 (s, 1H), 7.61 (s, 1H), 7.02-7.00 (m, 2H), 6.75 (s, 1H), 5.66-5.64 (m, 1H), 3.75-3.64 (m, 2H), 3.55 (s, 3H), 2.57 (s, 3H), 2.55-1.73 (m, 9H), 1.68 (d, J = 7.2 Hz, 3H), 1.35 (d, J = 6.8 Hz, 6H). |
| 92 | 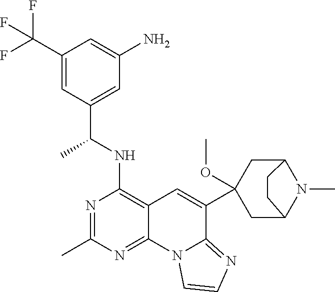 [SMILES: COC1(c2cc3c(N[C@H](C)c4cc(N)cc(C(F)(F)F)c4)nc(C)nc3n3ccnc23)CC2CCC(C1)N2C] | LC-MS[M + H]⁺: m/z 540.2. ¹H NMR (400 MHz, CD₃OD): δ 8.38 (s, 1H), 8.02 (s, 1H), 7.63 (s, 1H), 6.97-6.89 (m, 2H), 6.75 (s, 1H), 5.66-5.64 (m, 1H), 3.70-3.64 (m, 2H), 3.58 (s, 3H), 2.57 (s, 3H), 2.55-1.76 (m, 11H), 1.68 (d, J = 7.2 Hz, 3H). |
| 93 | 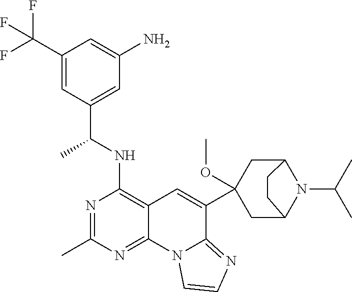 [SMILES: COC1(c2cc3c(N[C@H](C)c4cc(N)cc(C(F)(F)F)c4)nc(C)nc3n3ccnc23)CC2CCC(C1)N2C(C)C] | LC-MS[M + H]⁺: m/z 568.2. ¹H NMR (400 MHz, CD₃OD): δ 8.38 (s, 1H), 8.02 (s, 1H), 7.63 (s, 1H), 6.97-6.89 (m, 2H), 6.75 (s, 1H), 5.66-5.64 (m, 1H), 3.70-3.64 (m, 2H), 3.58 (s, 3H), 2.57 (s, 3H), 2.55-1.76 (m, 9H), 1.68 (d, J = 7.2 Hz, 3H), 1.27 (d, J = 6.8 Hz, 6H). |

### Example 94: (R)-5-(4-((1-(3-amino-5-(trifluoromethyl) phenyl) ethyl) amino)-2,8-dimethylimidazole [1′,2′:1,6] pyrido [2,3-d] pyrimidin-6-yl)-1-methylpyridin-2 (1H)-one

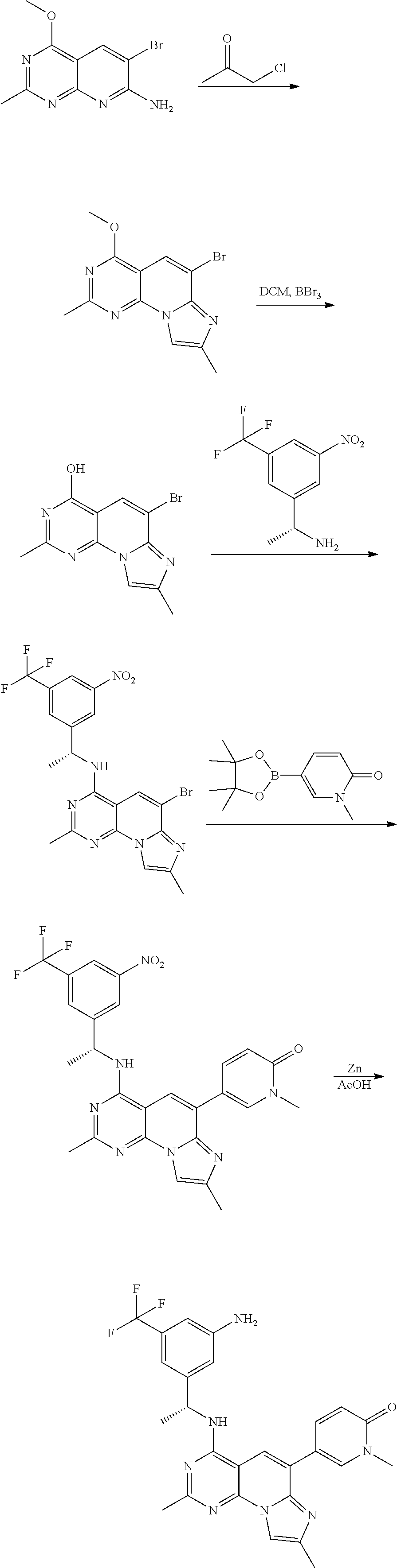

Step 1: Compound 6-bromo-4-methoxy-2 methylimidazole [1′,2′:1,6] pyrido [2,3-d]pyrimidin-7-amine (400 mg, 1.5 mmol) was dissolved in chloroacetone (10.0 mL). Under nitrogen protection, the reaction mixture was heated to 80 degree overnight. The reaction solution was concentrated under reduced pressure, and the crude product was purified by reverse phase column chromatography to give a yellow intermediate compound (300 mg). LC-MS[M+H]⁺: m/z 309.1.

Step 2: BBr₃ (2.6 mL, 2.6 mmol) was added to the above intermediate compound (160 mg, 0.52 mmol) in dichloromethane (5 mL) under nitrogen protection. The reaction solution was stirred overnight at room temperature. A saturated solution of ammonium chloride (1 mL) was added to quench the reaction, the reaction solution was concentrated under reduced pressure, and the obtained crude product was purified by reverse phase chromatography column to give a white product intermediate compound (71 mg). LC-MS[M+H]⁺: m/z 293.0.

Step 3: The above intermediate compound (70 mg, 0.24 mmol), (R)-1-(3-nitro-5-(trifluoromethyl) phenyl) ethyl-1-amine (70.6 mg, 0.30 mmol), BOP (159.2 mg, 0.36 mmol), DBU (82.1 mg, 0.36 mmol) were dissolved in DMF (50 mL). Under nitrogen protection, reaction solution was stirred overnight at room temperature. The reaction solution was diluted with water (100 mL) and extracted three times with ethyl acetate (100 mL). The combined organic phase was dried over anhydrous sodium sulfate and concentrated under reduced pressure. The resulting crude product was purified by silica gel column chromatography (eluent: petroleum ether/ethyl acetate=3:1) to give a yellow intermediate compound (41 mg). LC-MS[M+H]⁺: m/z 511.2.

Step 4: Under nitrogen protection, the above intermediate compound (40.0 mg, 0.08 mmol) and 1-methyl-5-(4,4,5,5-tetramethyl-1,3,2-dioxaborolane-2-yl) pyridin-2(1H)-one (23.0 mg, 0.096 mmol) were dissolved in a mixed solution of 1,4-dioxane and water (20 mL/4 mL), K₃PO₄ (51.0 mg, 0.24 mmol) and Pd(dtbpf)Cl₂ (5.0 mg, 0.008 mmol) were added. Under nitrogen protection, the reaction mixture was heated to 85 degrees and stirred overnight. LC-MS detection showed that the reaction was completed. The reaction solution was filtered through celite, and the filtrate was concentrated under reduced pressure. The resulting crude product was purified by silica gel column chromatography (eluent: petroleum ether/ethyl acetate=2/1) to give the intermediate compound as a white solid (25.0 mg). LC-MS[M+H]⁺: m/z 538.2.

Step 5: the above intermediate compound (40.0 mg, 0.08 mmol) was dissolved in acetic acid (5 mL), and zinc powder (156.0 mg, 2.4 mmol) was added. Under nitrogen protection, the reaction solution was heated to 60 degrees and stirred for 5 hours. The reaction solution was filtered, and the filtrate was concentrated under reduced pressure. The obtained crude product was purified by HPLC preparation to give white compound of Example 94 (7.1 mg). LC-MS[M+H]⁺: m/z 508.15. ¹H NMR (400 MHz, MeOD-d₄): δ 8.58 (s, 1H), 8.12-8.16 (m, 2H), 8.07 (s, 1H), 6.99 (d, J=8.0 Hz, 2H), 6.80 (s, 1H), 6.70 (d, J=9.2 Hz, 2H), 5.61-5.63 (m, 1H), 3.70 (s, 3H), 2.55 (s, 3H), 2.46 (s, 3H), 1.65 (d, J=6.8 Hz, 3H).

### Example 95: (R)-N-(1-(3-Amino-5-(trifluoromethyl) phenyl) ethyl)-2,8-dimethyl-6-morpholinimidazole [1′,2′:1,6] pyrido [2,3-d] pyrimidin-4-amine

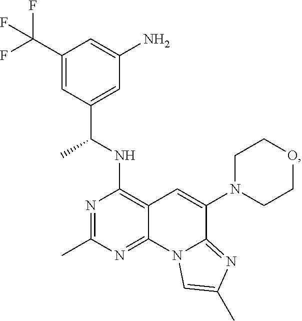 [SMILES: Cc1cn2c(n1)c(N1CCOCC1)cc1c(N[C@H](C)c3cc(N)cc(C(F)(F)F)c3)nc(C)nc12]

example 95 was obtained by referring to the synthesis methods of examples 69 and 94. LC-MS[M+H]⁺: m/z 486.1. ¹H NMR (400 MHz, MeOD-d4): δ 8.10 (s, 1H), 7.25 (s, 1H), 6.99-7.02 (m, 2H), 6.82 (s, 1H), 5.62-5.65 (m, 1H), 3.97-4.00 (m, 4H) 3.40-3.41 (m, 4H), 2.54 (s, 3H), 2.46 (s, 3H), 1.66 (d, J=8.4 Hz, 3H).

### Example 96: (R)-1-(4-(4-((1-(3-amino-5-(trifluoromethyl) phenyl) ethyl) amino)-2,8-dimethylimidazole [1′,2′:1,6] pyrido [2,3-d] pyrimidin-6-yl)-4-methoxypiperidin-1-yl) ethyl-1-one

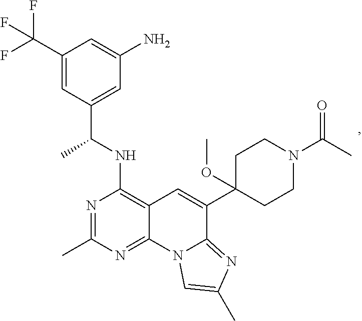 [SMILES: COC1(c2cc3c(N[C@H](C)c4cc(N)cc(C(F)(F)F)c4)nc(C)nc3n3cc(C)nc23)CCN(C(C)=O)CC1]

example 96 was obtained by referring to the synthesis methods of examples 75 and 94. LC-MS[M+H]⁺: m/z 556.2. ¹H NMR (400 MHz, MeOD-d4): δ 8.10 (s, 1H), 7.25 (s, 1H), 6.99-7.02 (m, 1H), 6.82 (s, 1H), 5.62-5.65 (m, 1H), 4.59-4.56 (m, 1H), 3.90-3.89 (m, 1H), 3.77-3.74 (m, 1H), 3.55 (s, 3H), 3.25-3.23 (m, 1H), 2.57 (s, 3H), 2.55-2.52 (m, 2H), 2.46 (s, 3H), 2.20 (s, 3H), 2.07-1.96 (m, 2H), 1.68 (d, J=6.8 Hz, 3H).

### Test Example 1 Test of Enzyme Inhibitory Activity

Compounds were tested for their efficacy in inhibiting the protein-protein interaction between SOS1 and KRAS^{G12C} with the KRAS^{G12C}/SOS1 kit from CisBio, using the Binding assay method, and the results were expressed as IC₅₀ values.

Test method: (1) The test concentration of the tested compound was 1000 nM, and which was diluted to a 200-fold final concentration in 100% DMSO in a 384-well plate, the compound was diluted 3-fold for 10 concentrations. The dispenser Echo 550 was used to transfer 50 nL of 200-fold final concentration of compound to the target plate the 384 well plate. 50 nL of 100% DMSO was added to negative control wells and positive control wells, respectively; (2) 4 times the final concentration of Tag1 SOS1 solution was prepared with Diluent buffer; (3) 2.5 μL of 4 times the final concentration of Tag1 SOS1 solution was added into 384 well plate; (4) 4 times the final concentration of Tag2 KRAS^{G12C} solution was prepared with Diluent buffer; (5) 2.5 μL of the 4 times the final concentration of Tag2 KRAS^{G12C} solution was added to the compound wells and the positive control well respectively; 2.5 μL of diluent buffer was added to the negative control well; (6) the 384 well plate was centrifuged at 1000 rpm for 30 seconds, and incubated at room temperature for 15 min after shaking and mixing; (7) 1 times the final concentration of Anti Tag1 TB3+solution and 1 times the final concentration of Anti Tag2 XL665 solution were prepared with Detection buffer. After mixing the two solutions, 5 μL of mixed solution was added to each wells; (8) the 384 well plate was centrifuged at 1000 rpm for 30 seconds, shaken and mixed well, then incubated at room temperature for 120 minutes; (9) Em665/620 was read with Envision ELIASA; (10) Data analysis, calculation formula

$$Inhibition \% = \frac{Max signal - Compound signal}{Max signal - Min signal} \times 100 ;$$

wherein, Min signal means the mean value of negative control wells and Max signal means the mean value of positive control wells. The fitted dose-effect curve is based on the log value of concentration as the X axis, the percentage inhibition rate as the Y axis, and the log(inhibitor) vs. response Variable slope of the analysis software GraphPad Prism 5 is used to fit the dose-effect curve, so as to obtain the IC₅₀ value of each compounds on enzyme activity. The fitting formula is: Y=Bottom+(Top Bottom)/(1+10\hat{ }((Log IC₅₀X)*HillSlope)).

Results: most of the example compounds of the present invention showed high inhibitory activity against KRAS^{G12C}/SOS1 enzyme, with an IC₅₀ of less than 100 nM, and some of the examples showed an IC₅₀ value of less than 50 nM. (The range of IC₅₀ values is expressed as follows: A<50 nM, 50 nM<B<100 nM, C>100 nM).

| | KRAS^{G12C}/ | | KRAS^{G12C}/ | | KRAS^{G12C}/ |
|---|---|---|---|---|---|
| | SOS1 | | SOS1 | | SOS1 |
| No | IC₅₀ (nM) | No | IC₅₀ (nM) | No | IC₅₀ (nM) |
| AMG-510 | 37 | BI-3406 | 11 | 1 | 179 |
| 2 | 817 | 3 | 8.3 | 4 | A |
| 5 | A | 6 | A | 7 | A |
| 8 | 94 | 9 | 118 | 10 | A |
| 11 | A | 12 | 69 | 13 | A |
| 14 | A | 15 | A | 16 | A |
| 17 | A | 18 | A | 19 | A |
| 20 | A | 21 | 254 | 22 | C |
| 23 | 30.5 | 24 | A | 25 | A |
| 26 | C | 27 | A | 28 | A |
| 29 | B | 30 | B | 31 | 187 |
| 32 | 173 | 33 | C | 34 | 32 |
| 35 | A | 36 | 22 | 37 | A |
| 38 | A | 39 | 42 | 40 | A |
| 41 | A | 42 | 34 | 43 | 48 |
| 44 | 385 | 45 | C | 46 | C |
| 47 | C | 48 | 330 | 49 | 172 |
| 50 | 17 | 51 | A | 52 | A |
| 53 | A | 54 | A | 55 | B |
| 56 | 10 | 57 | B | 58 | B |
| 59 | A | 60 | 10 | 61 | A |
| 62 | 24 | 63 | C | 64 | B |
| 65 | A | 66 | A | 67 | A |
| 68 | B | 69 | 232 | 70 | B |
| 71 | 166 | 72 | 273 | 73 | 199 |
| 74 | 301 | 75 | A | 76 | A |
| 77 | A | 78 | A | 79 | A |
| 80 | A | 81 | A | 82 | 189 |
| 83 | 245 | 84 | 251 | 85 | 3.5 |
| 86 | A | 87 | A | 88 | A |
| 89 | A | 90 | A | 91 | A |
| 92 | A | 93 | A | 94 | C |
| 95 | B | 96 | A | | |

### Test Example 2: Effect of Example Compounds on MiaPaca-2 Cell Proliferation

Test Method One (2D): MiaPaca-2 (pancreatic cancer) cells (100 μL/well, 20000 cells/mL) were seeded in 96-well culture plates and supplemented with 1000 fetal bovine serum and 1% penicillin/streptomycin sulfate. Using 0.5% dimethyl sulfoxide as a blank, the cells were treated with a solution of the test compound at a starting concentration of 10 μM in eight steps of three-fold dilution and incubated in a 500 CO2 incubator for a certain period of time (5-7 days). At the end of the incubation, 10 μL of MTT stock solution (5 mg/mL) was added to each well. The plates were incubated at 37° C. for 4 hours and then the medium was removed. Dimethyl sulfoxide (100 μL) was added to each well and then shaken well. The absorbance of the formazan product was measured at 570 nm on a Thermo Scientific Varioskan Flash multimode reader. IC₅₀ values were obtained by fitting the dose response data to a three-parameter non-linear regression model using GraphPad Prism 6.0 software.

### Test Example 3: Effect of Example Compounds on p-ERK Ability

PC9 cells were seeded in 384-well cell culture plate (40 μL/well) according to a certain concentration and placed in a cell culture incubator with 37° C. and 5% CO₂ overnight. On the next day, the plate was added with serially diluted test compounds (5 concentrations, 3-fold dilution, the highest concentration was 10 uM) for 1 hour, and then the lysate containing protease and phosphatase inhibitor was added to lyse the cells-extracted proteins, cellular pERK levels were measured using the AlphaLISASureFire Ultra p ERK1/2 assay kit (PerkinElmer). The standard AlphaLISA settings on the Envision plate reader (PerkinElmer) was used to read the signal. Raw data were analyzed in Excel (Microsoft) and Prism (GraphPad). The signal was plotted against the common logarithm of compound concentration and IC₅₀ was calculated by fitting a four parameter non-linear regression curve.

Results: the SOS1 pERK IC₅₀ of most of the compounds of the present invention is less than 5 uM, and the SOS1 pERK IC₅₀ of some compounds is less than 1 uM, such as compounds 75, 76, 77, 78, 79, 80, 81, 85, 89, 90, 91, 92, 93, etc.

All literatures mentioned in the present invention are incorporated by reference herein, as though individually incorporated by reference. Additionally, it should be understood that after reading the above teaching, many variations and modifications may be made by the skilled in the art, and these equivalents also fall within the scope as defined by the appended claims.

1. A pyrimido heterocyclic compound of formula (I-1) or (I-2), or a pharmaceutically acceptable salt thereof, or an enantiomer, a diastereomer, a tautomer, a torsional isomer, a solvate, a polymorph or a prodrug thereof, 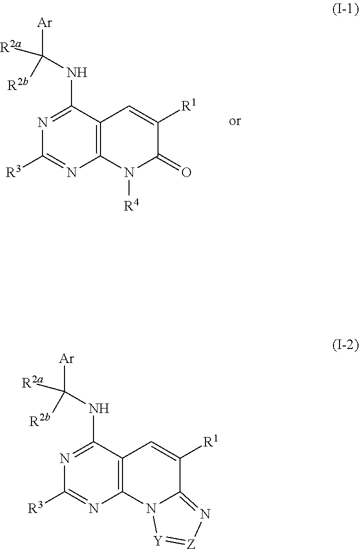
wherein:
R¹ is independently selected from C₁-C₁₀ alkyl, C₁-C₁₀ haloalkyl, C₂-C₁₀ alkenyl, C₂-C₁₀ alkynyl, C₃-C₁₂ cycloalkyl, C₄-C₁₂ cycloalkenyl, 3-12 membered heterocycloalkyl, 5-12 membered aryl or 5-12 membered heteroaryl, —OC₁-C₁₀ alkyl, —NHC₁-C₁₀ alkyl, —N (C₁-C₁₀ alkyl)(C₁-C₁₀ alkyl), —NH (C₃-C₁₂ cycloalkyl), —NH (3-12 membered heterocycloalkyl), —O(C₃-C₁₂ cycloalkyl), —O (3-12 membered heterocycloalkyl), —SC₁-C₁₀ alkyl, —SOC₁-C₁₀ alkyl, —SO₂C₁-C₁₀ alkyl, carbocycle or heteroatom-containing spiro ring/bridged ring/fused ring, wherein, the C₁-C₁₀ alkyl, C₂-C₁₀ alkenyl, C₂-C₁₀ alkynyl, C₃-C₁₂ cycloalkyl, C₄-C₁₂ cycloalkenyl, 3-12 membered heterocycloalkyl, 5-12 membered aryl or 5-12 membered heteroaryl, carbocycle or heteroatom-containing spiro ring/bridged ring/fused ring may be optionally substituted with 1-3 Rn; or two of the above Rn may form 3-12 membered saturated or partially unsaturated, or aromatic ring system through carbon chain or heteroatom; Rn is independently selected from hydrogen, deuterium, halogen, cyano, nitro, amido, sulfonamido, hydroxyl, amino, ureido, phosphoryl, alkylphosphoxy, alkylsilyl, C₁-C₆ alkyl, C₁-C₆ alkoxy, haloalkyl, haloalkoxy, C₁-C₆ monoalkylamino, C₁-C₆ dialkylamino, alkenyl, alkynyl, 3-8 membered cycloalkyl or heterocycloalkyl, C₁-C₆ alkyl-S—, C₁-C₆ alkyl-SO—, C₁-C₆ alkyl-SO₂—, and the like;
R^{2a} and R^{2b} are each independently selected from hydrogen, deuterium, halogen, C₁-C₆ alkyl, 3-8 membered cycloalkyl or heterocycloalkyl; and R^{2a} and R^{2b} or R^{2a} and substituent R^{m} on Ar may form a 3-8 membered saturated or partially unsaturated or unsaturated ring system through a carbon chain or heteroatom;
R³ and R⁴ are each independently H, deuterium, halogen, hydroxyl, amino, cyano, C₁-C₆ alkyl, C₁-C₆ haloalkyl, C₁-C₆ alkoxy, C₁-C₆ haloalkoxy, C₁-C₆ alkylamino, 3-8 membered cycloalkyl or heterocycloalkyl, C₂-C₄ alkenyl, C₂-C₄ alkynyl, 5-10 membered aromatic ring or aromatic heterocyclyl;
Y and Z are each independently selected from N or CR⁵, R⁵ is selected from hydrogen, deuterium, halogen, cyano, C₁-C₆ alkyl, 3-8 membered cycloalkyl or heterocycloalkyl;
Ar is selected from 5-12 membered monocyclic or bicyclic aryl or heteroaryl, which may be substituted by one or more R^{m}, R^{m} is selected from the group consisting of
hydrogen, deuterium, halogen, cyano, nitro, substituted or unsubstituted amido, substituted or unsubstituted sulfonamido, hydroxyl, amino, ureido, phosphoryl, alkylphosphoxy, alkylsilyl, C₁-C₁₀ alkyl, C₁-C₁₀ alkoxy, C₁-C₁₀ alkoxyalkyl, C₁-C₁₀ haloalkyl, C₁-C₁₀ haloalkoxy, C₁-C₁₀ haloalkoxyalkyl, C₁-C₁₀ monoalkylamino, C₁-C₁₀ dialkylamino, C₁-C₁₀ monoalkylaminoalkyl, C₁-C₁₀ dialkylaminoalkyl, C₁-C₁₀ alkenyl, C₁-C₁₀ alkynyl, 3-12 membered cycloalkyl or heterocycloalkyl, 3-12 membered cycloalkyl or heterocycloalkyl alkyl, C₁-C₁₀ alkyl-S—, C₁-C₁₀ alkyl-SO—, C₁-C₁₀ alkyl-SO₂—, substituted or unsubstituted 5-12 membered aryl or heteroaryl, and the like, or two of the above R^{m} may form a 3-12 membered saturated or partially unsaturated, or aromatic ring system through a carbon chain or heteroatom;
one or more hydrogen atoms on any of the above groups may be substituted by a substituent selected from the group consisting of (including but not limited to) deuterium, halogen, hydroxyl, C₁-C₃ alkyl, C₁-C₃ haloalkyl, 3-6 membered cycloalkyl or heterocycloalkyl, oxo (═O), C₁-C₃ alkoxy, C₁-C₁₀ monoalkylaminoalkyl, C₁-C₁₀ dialkylaminoalkyl, 3-6 membered cycloalkyl C₁-C₃ alkyl-, 3-6 membered heterocycloalkyl C₁-C₃ alkyl-, amino 3-6 membered cycloalkyl-, amino 3-6 membered heterocycloalkyl-, C(═O)(3-6 membered heterocyclyl) C₁-C₃ alkyl, C(═O)C₁-C₃ alkyl, C(═O)C₁-C₁₀ monoalkylaminoalkyl, C(═O) C₁-C₁₀ dialkylaminoalkyl, C(═O)C₁-C₃ alkyl, C(═O) amino C₁-C₁₀ monoalkylOH, C(═O) amino C₁-C₁₀ dialkylOH;
wherein, the heteroaryl contains 1-3 heteroatoms selected from the group consisting of N, O, P or S, the heterocycloalkyl contains 1-3 heteroatoms selected from the group consisting of N, O, P or S, and the ring system comprises a saturated or partially unsaturated ring system such as spiro ring, bridged ring, fused ring, and fused ring.

2. The compound of claim 1, which is a compound of formula (II-1) or (II-2), or a pharmaceutically acceptable salt thereof, or an enantiomer, a diastereomer, a tautomer, a torsional isomer, a solvate, a polymorph, or a prodrug thereof: 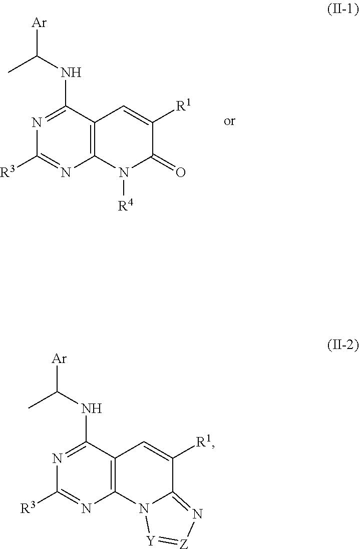
wherein, R¹, R³, R⁴, Ar, Y and Z are as defined in claim 1.

3. The compound of claim 1, which is a compound of formula (III-1) to (III-12), or a pharmaceutically acceptable salt thereof, or an enantiomer, a diastereomer, a tautomer, a torsional isomer, a solvate, a polymorph, or a prodrug thereof: 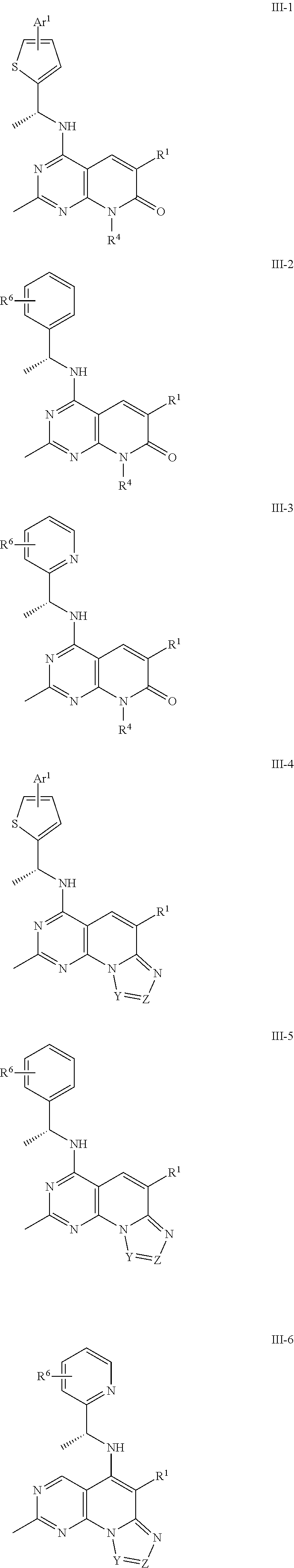 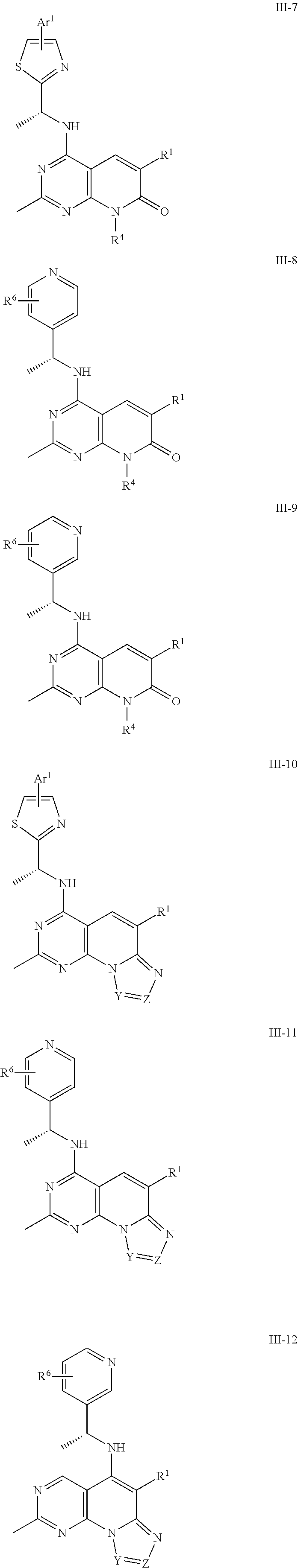
wherein, Ar¹ is preferably a 5-6 membered aromatic or heteroaromatic ring system, and the above ring system may be substituted by 1-5 substituents selected from the group consisting of hydrogen, deuterium, halogen, cyano, nitro, substituted or unsubstituted amido, substituted or unsubstituted sulfonamido, hydroxyl, amino, ureido, phosphoryl, alkylphosphoxy, alkylsilyl, C₁-C₁₀ alkyl, C₁-C₁₀ alkoxy, C₁-C₁₀ alkoxyalkyl, C₁-C₁₀ haloalkyl, C₁-C₁₀ haloalkoxy, C₁-C₁₀ haloalkoxyalkyl, C₁-C₁₀ monoalkylamino, C₁-C₁₀ dialkylamino, C₁-C₁₀ monoalkylaminoalkyl, C₁-C₁₀ dialkylaminoalkyl, C₁-C₁₀ alkenyl, C₁-C₁₀ alkynyl, 3-12 membered cycloalkyl or heterocycloalkyl, 3-12 membered cycloalkyl or heterocycloalkyl alkyl, C₁-C₁₀ alkyl-S—, C₁-C₁₀ alkyl-SO—, C₁-C₁₀ alkyl-SO₂—, substituted or unsubstituted 5-12 membered aryl or heteroaryl, and the like; R⁶ is independently selected from 1-5 substituents selected from the group consisting of hydrogen, deuterium, halogen, cyano, nitro, substituted or unsubstituted amido, substituted or unsubstituted sulfonamido, hydroxyl, amino, ureido, phosphoryl, alkylphosphoxy, alkylsilyl, C₁-C₁₀ alkyl, C₁-C₁₀ alkoxy, C₁-C₁₀ alkoxyalkyl, C₁-C₁₀ haloalkyl, C₁-C₁₀ haloalkoxy, C₁-C₁₀ haloalkoxyalkyl, C₁-C₁₀ monoalkylamino, C₁-C₁₀ dialkylamino, C₁-C₁₀ monoalkylaminoalkyl, C₁-C₁₀ dialkylaminoalkyl, C₁-C₁₀ alkenyl, C₁-C₁₀ alkynyl, 3-12 membered cycloalkyl or heterocycloalkyl, 3-12 membered cycloalkyl or heterocycloalkyl alkyl, C₁-C₁₀ alkyl-S—, C₁-C₁₀ alkyl-SO—, C₁-C₁₀ alkyl-SO₂—, substituted or unsubstituted 5-12 membered aryl or heteroaryl, and the like; R¹, R⁴, Y and Z are as defined in claim 1.

4. The compound of claim 1 or a pharmaceutically acceptable salt thereof, or an enantiomer, a diastereomer, a tautomer, a torsional isomer, a solvate, a polymorph or a prodrug thereof, wherein R¹ is selected from the group consisting of 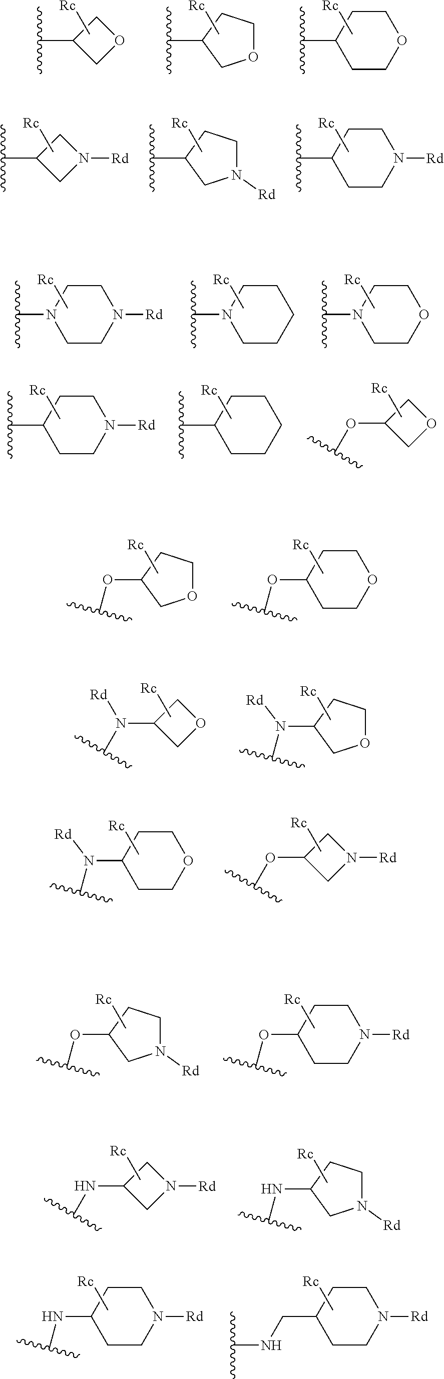 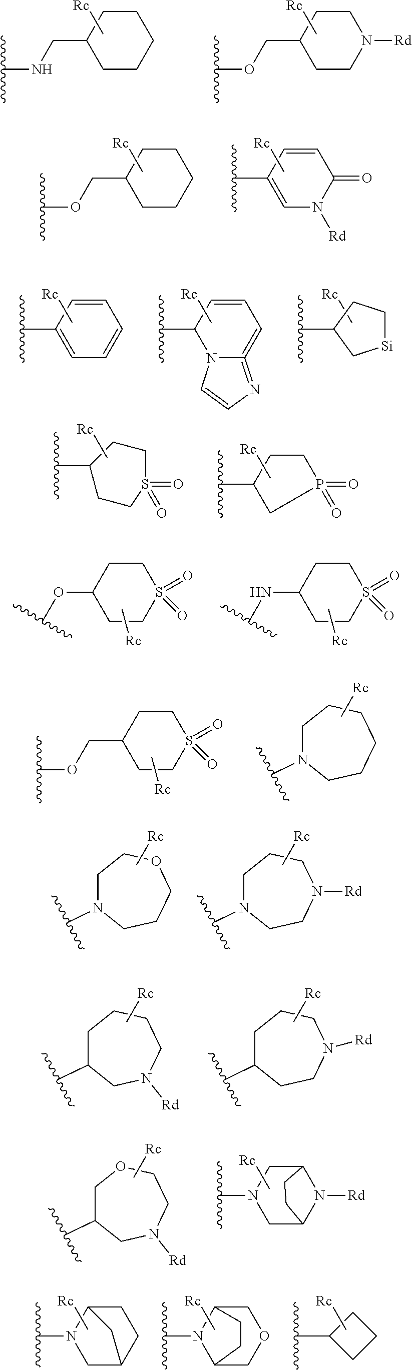 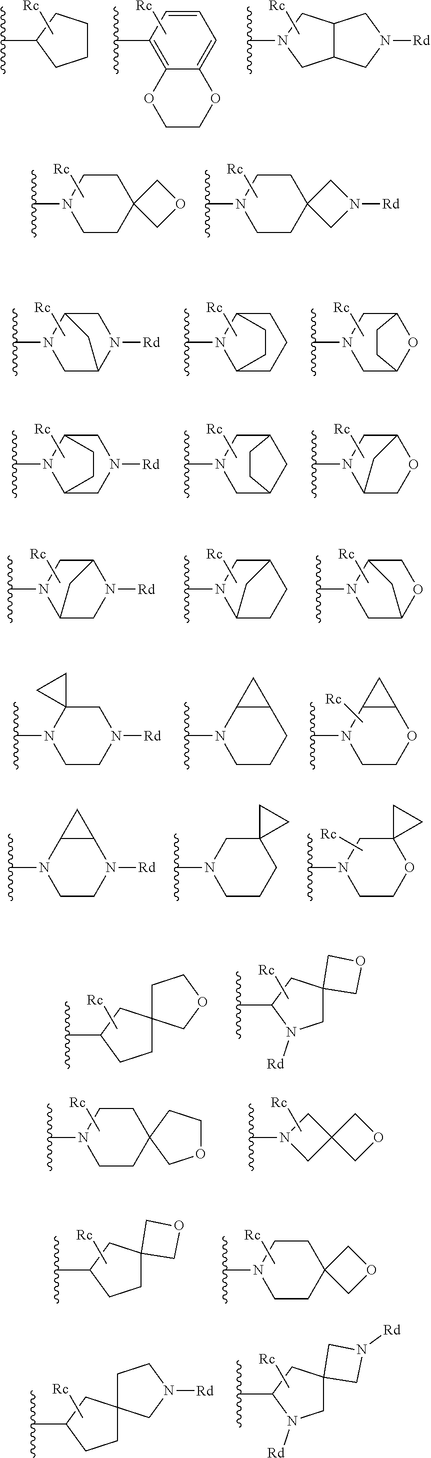 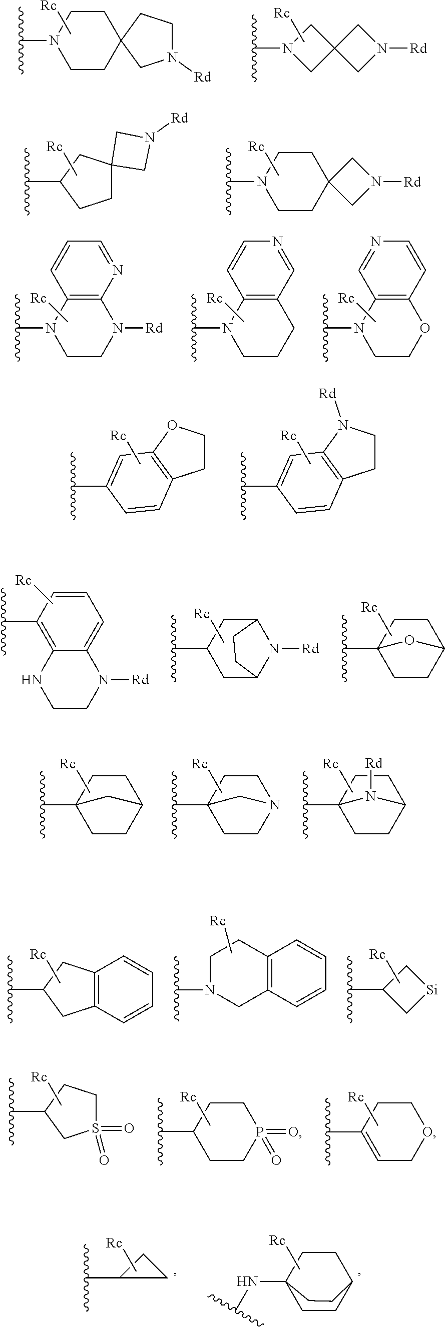
wherein one or more R^{c} are each independently selected from hydrogen, deuterium, halogen, —C₁-C₆ alkyl, —OC₁-C₆ alkyl, cyano, hydroxy, amino, —SC₁-C₆ alkyl, —SOC₁-C₆ alkyl, —SO₂C₁-C₆ alkyl, —COC₁-C₆ alkyl, —COOC₁-C₆ alkyl, —CONHC₁-C₆ alkyl, —CON (C₁-C₆ alkyl) (C₁-C₆ alkyl), 3-6 membered cycloalkyl or heterocycloalkyl, 5-10 membered aryl or heteroaryl, —C₁-C₆ haloalkyl, —C₁-C₆ haloalkoxy, —C₁-C₆ deuterated alkyl, —C₁-C₆ deuterated alkoxy, —O-3-6 membered cycloalkyl or heterocycloalkyl, —C₁-C₆ alkyl OC₁-C₆ alkyl, —C₁-C₆ alkyl NHC₁-C₆ alkyl, —C₁-C₆ alkyl OH, —C₁-C₆ alkyl N (C₁-C₆ alkyl) (C₁-C₆ alkyl), —C₁-C₆ alkyl 3-6 membered cycloalkyl, —C₁-C₆ alkyl 3-6 membered heterocycloalkyl, C(═O)(3-6 membered heterocyclyl) C₁-C₃ alkyl, or C(═O) amino C₁-C₆ dialkyl OH, and any two of Rc can form 3-10 membered saturated or partially unsaturated carbocyclic or heterocyclic ring through carbon chain or heteroatom; R^{d} is independently selected from —C₁-C₆ alkyl, —C₁-C₆ alkyl OC₁-C₆ alkyl, —C₁-C₆ alkyl SC₁-C₆ alkyl, —C₁-C₆ alkyl SOC₁-C₆ alkyl, —C₁-C₆ alkyl SO₂C₁-C₆ alkyl, —COC₁-C₆ alkyl, —COOC₁-C₆ alkyl, —CONHC₁-C₆ alkyl, —CON (C₁-C₆ alkyl) (C₁-C₆ alkyl), 3-6 membered cycloalkyl or heterocycloalkyl, 5-10 membered aryl or heteroaryl, —C₁-C₆ haloalkyl, —C₁-C₆ haloalkoxy, —C₁-C₆ deuterated alkyl, —C₁-C₆ deuterated alkoxy-C₁-C₆ alkyl, —C₁-C₆ alkyl-O-3-6-membered cycloalkyl or heterocycloalkyl, —C₁-C₆ alkyl-NHC₁-C₆ alkyl, —C₁-C₆ alkyl OH, —C₁-C₆ alkyl N (C₁-C₆ alkyl) (C₁-C₆ alkyl), and the like.

5. The compound of claim 1, or a pharmaceutically acceptable salt thereof, or an enantiomer, a diastereomer, a tautomer, a torsional isomer, a solvate, a polymorph, or a prodrug thereof, characterized in that the compound has a structure shown in Formula IV, 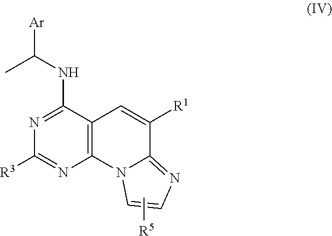
wherein, Ar, R¹, R³ and R⁵ are as defined in claim 1.

6. The compound of claim 1, or a pharmaceutically acceptable salt thereof, or an enantiomer, a diastereomer, a tautomer, a torsional isomer, a solvate, a polymorph or a prodrug thereof, wherein R¹ is selected from

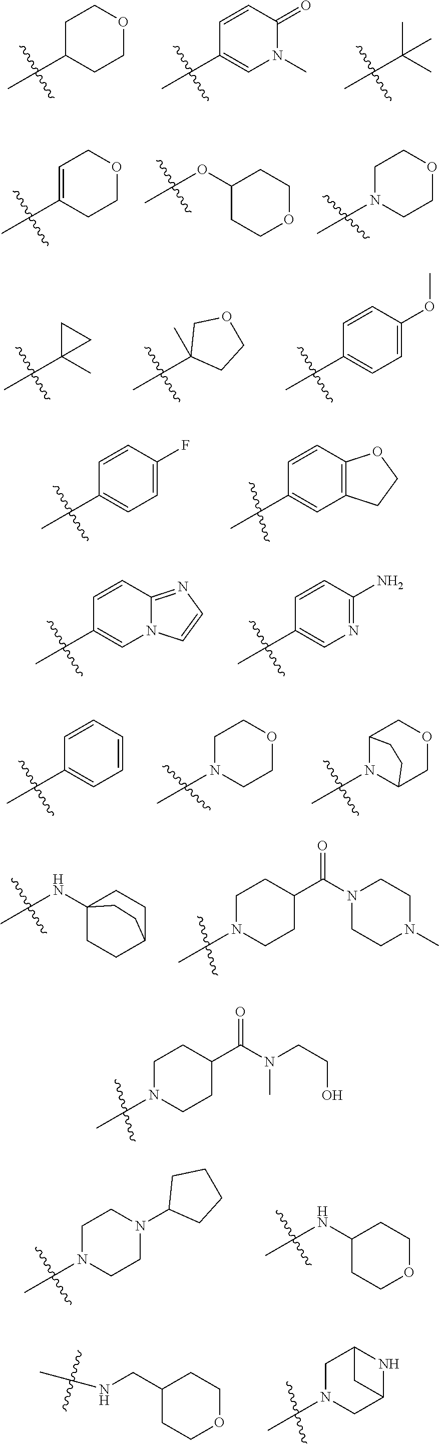

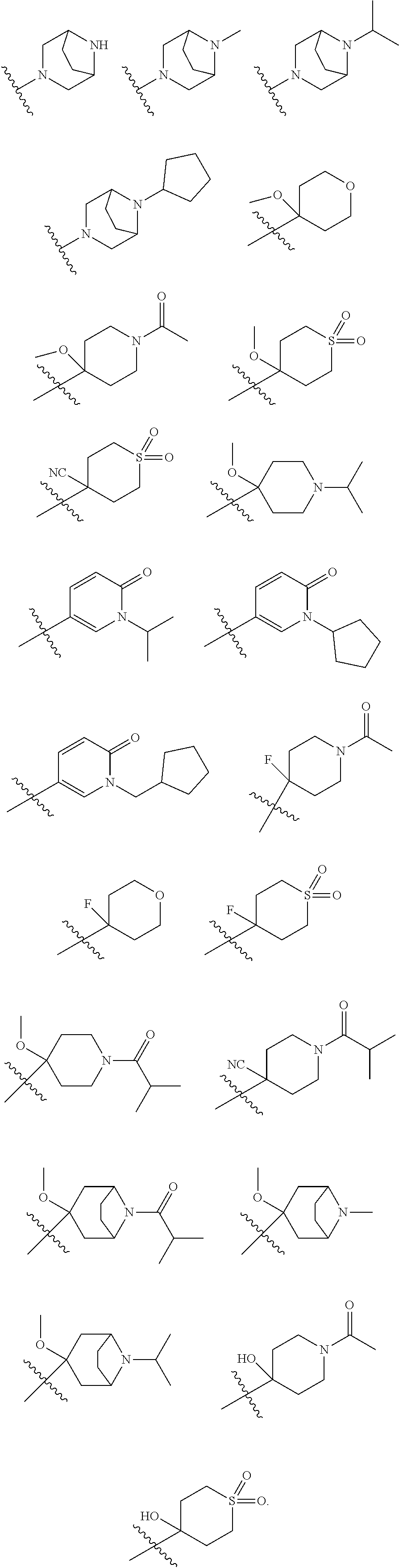

7. The compound of claim 1, or a pharmaceutically acceptable salt thereof, or an enantiomer, a diastereomer, a tautomer, a torsional isomer, a solvate, a polymorph, or a prodrug thereof, wherein 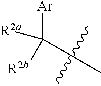
is selected from

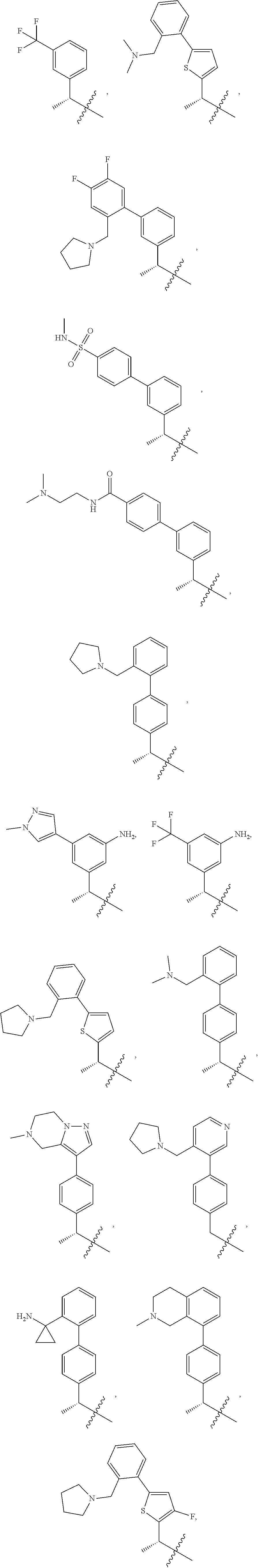

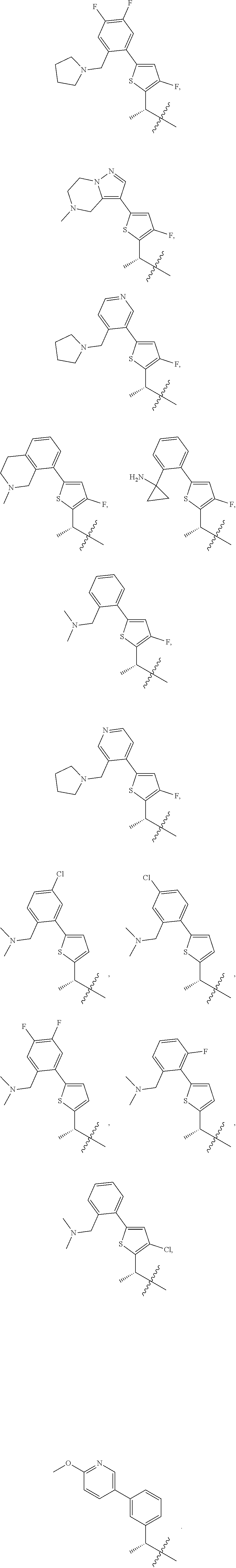

8. The compound of claim 1, or a pharmaceutically acceptable salt thereof, or an enantiomer, a diastereomer, a tautomer, a torsional isomer, a solvate, a polymorph, or a prodrug thereof, wherein the compound has the following structure:

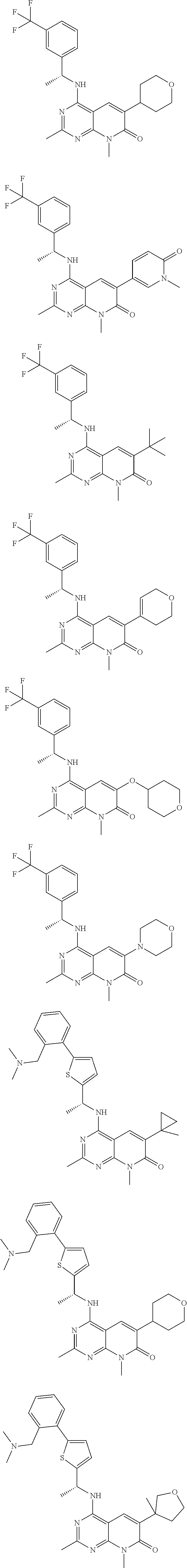

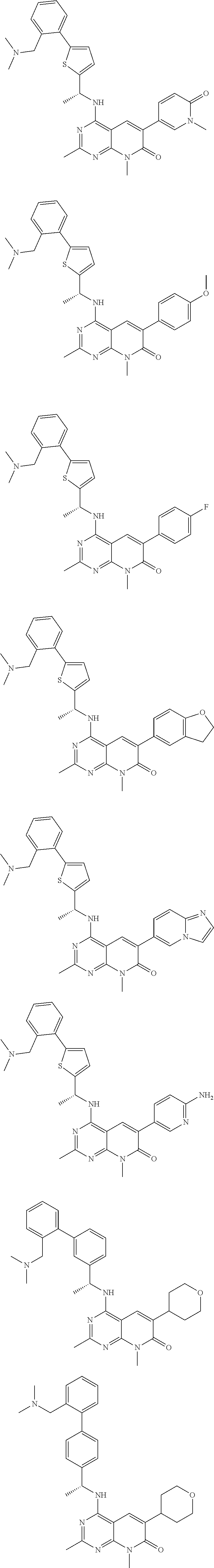

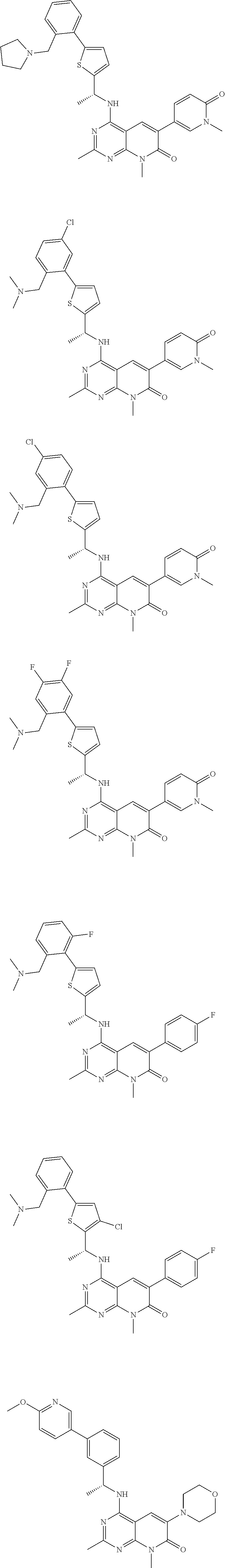

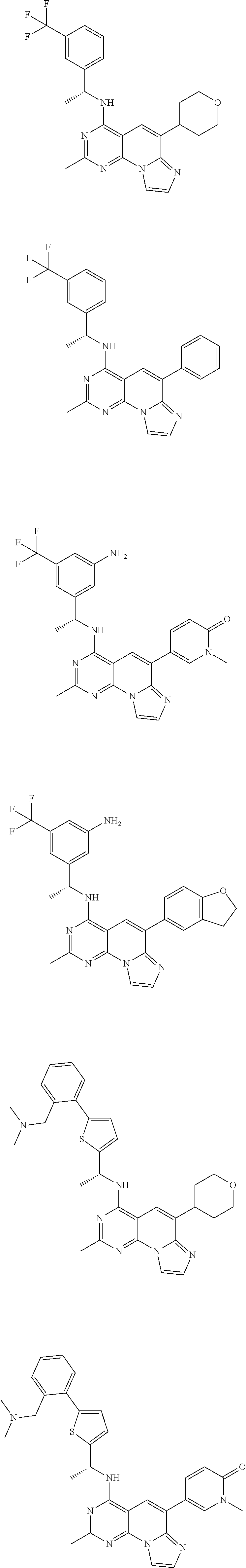

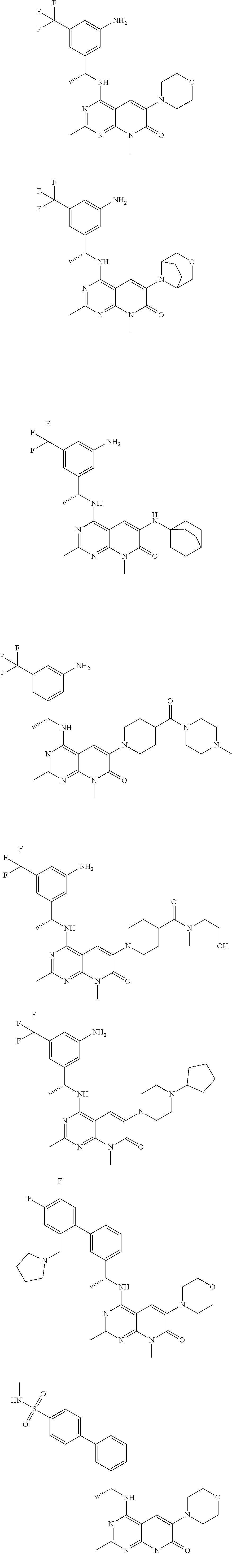

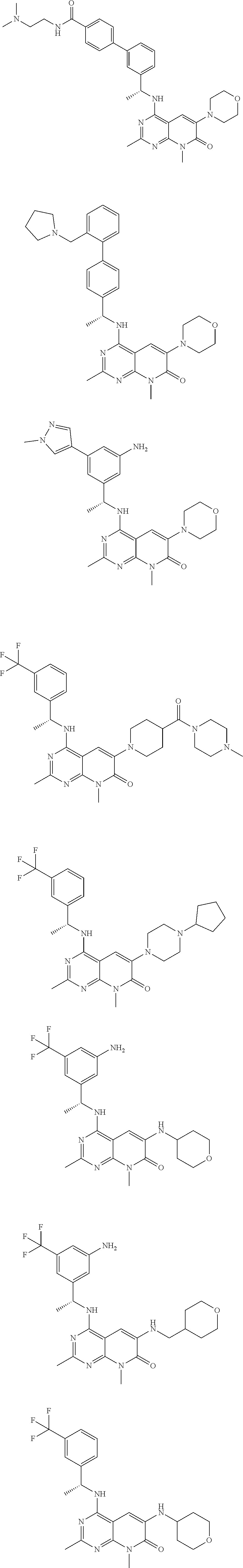

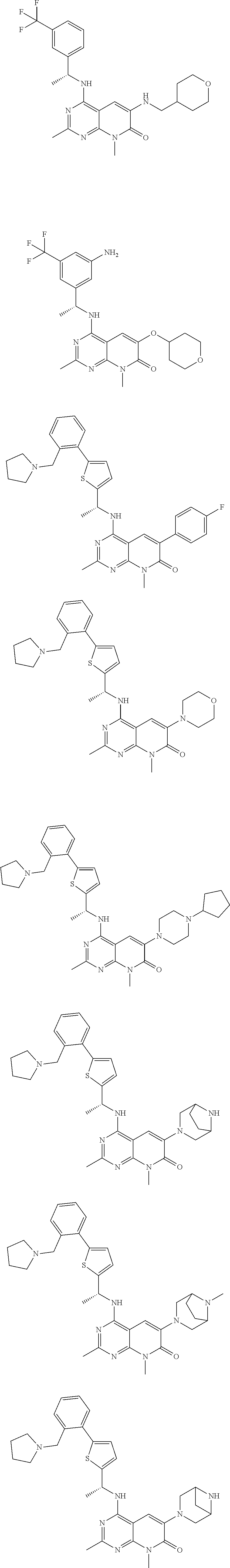

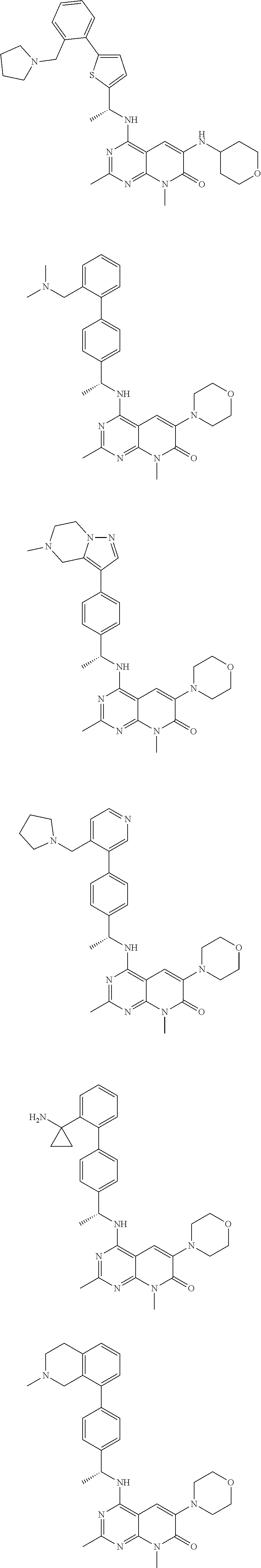 [SMILES: Cc1nc(N[C@H](C)c2ccc(-c3ccccc3CN3CCCC3)s2)c2cc(NC3CCOCC3)c(=O)n(C)c2n1 ; Cc1nc(N[C@H](C)c2ccc(-c3ccccc3CN(C)C)cc2)c2cc(N3CCOCC3)c(=O)n(C)c2n1 ; Cc1nc(N[C@H](C)c2ccc(-c3cnn4c3CN(C)CC4)cc2)c2cc(N3CCOCC3)c(=O)n(C)c2n1 ; Cc1nc(N[C@H](C)c2ccc(-c3cnccc3CN3CCCC3)cc2)c2cc(N3CCOCC3)c(=O)n(C)c2n1 ; Cc1nc(N[C@H](C)c2ccc(-c3ccccc3C3(N)CC3)cc2)c2cc(N3CCOCC3)c(=O)n(C)c2n1 ; Cc1nc(N[C@H](C)c2ccc(-c3cccc4c3CN(C)CC4)cc2)c2cc(N3CCOCC3)c(=O)n(C)c2n1]

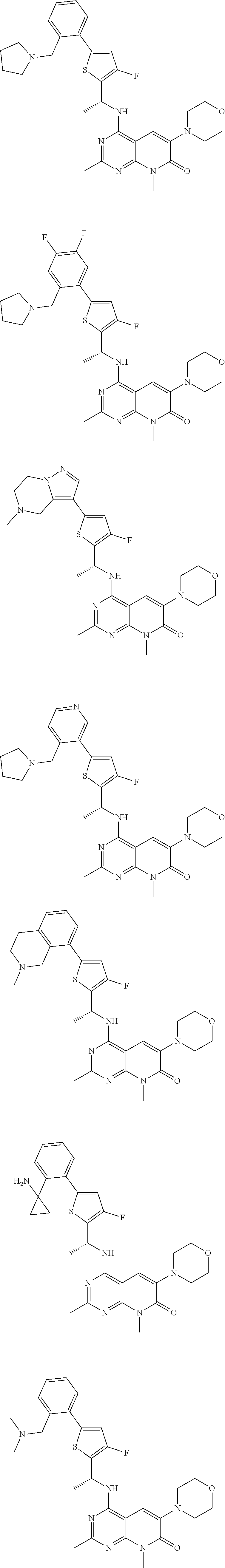

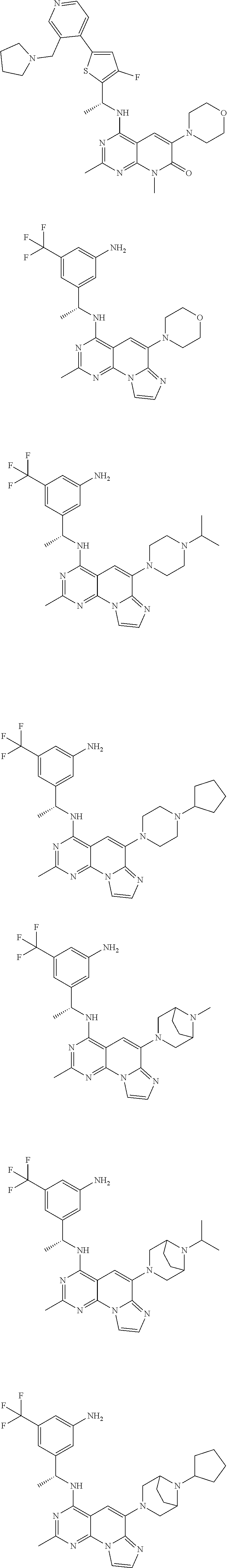

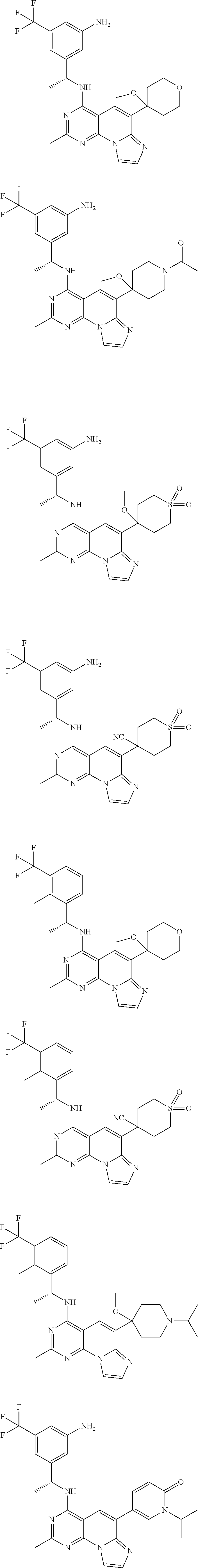

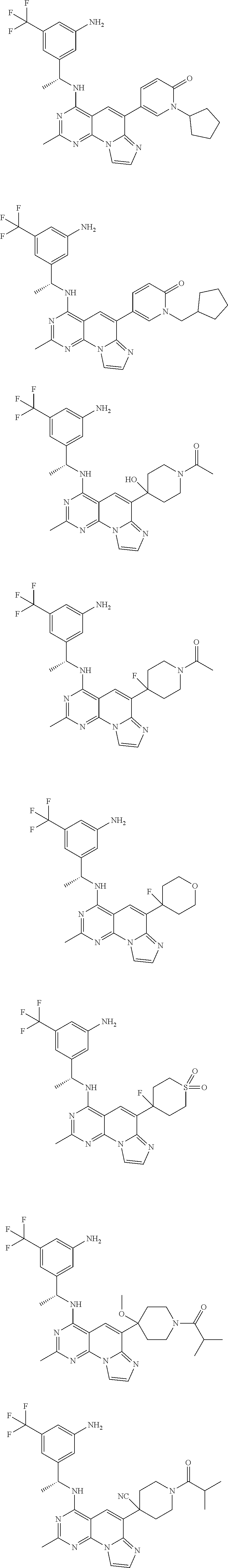

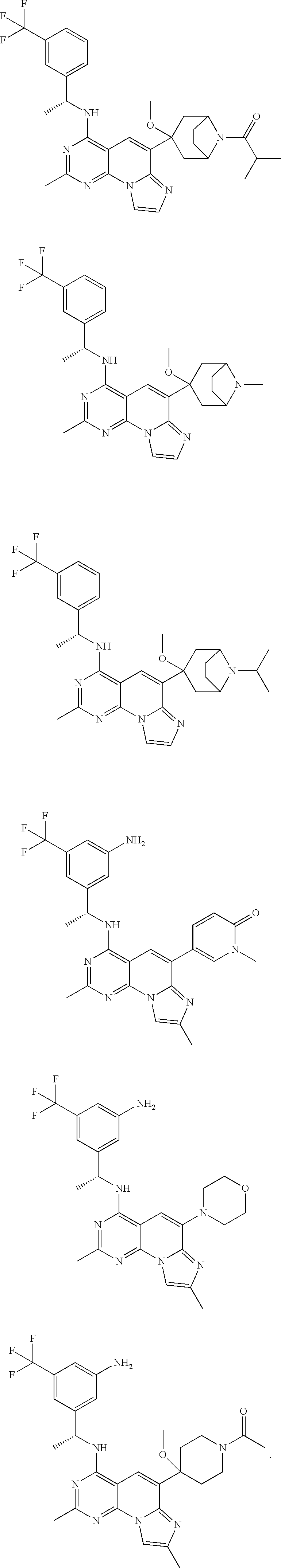 [SMILES: COC1(c2cc3c(N[C@H](C)c4cccc(C(F)(F)F)c4)nc(C)nc3n3ccnc23)CC2CCC(C1)N2C(=O)C(C)C ; COC1(c2cc3c(N[C@H](C)c4cccc(C(F)(F)F)c4)nc(C)nc3n3ccnc23)CC2CCC(C1)N2C ; COC1(c2cc3c(N[C@H](C)c4cccc(C(F)(F)F)c4)nc(C)nc3n3ccnc23)CC2CCC(C1)N2C(C)C ; Cc1cn2c(n1)c(-c1ccc(=O)n(C)c1)cc1c(N[C@H](C)c3cc(N)cc(C(F)(F)F)c3)nc(C)nc12 ; Cc1cn2c(n1)c(N1CCOCC1)cc1c(N[C@H](C)c3cc(N)cc(C(F)(F)F)c3)nc(C)nc12 ; COC1(c2cc3c(N[C@H](C)c4cc(N)cc(C(F)(F)F)c4)nc(C)nc3n3cc(C)nc23)CCN(C(C)=O)CC1]

9. Use of the compound of formula I according to claim 1 or a pharmaceutically acceptable salt thereof, or an enantiomer, a diastereomer, a tautomer, a torsional isomer, a solvate, a polymorph or a prodrug thereof for the preparation of a drug for the treatment of a disease associated with activity or expression or mutation of Ras protein, in particular a drug for the treatment of a tumor. The tumor is independently selected from lung cancer, pancreatic cancer, liver cancer, colorectal cancer, cholangiocarcinoma, brain cancer, leukemia, lymphoma, melanoma, thyroid cancer, nasopharyngeal cancer, and the like.

10. A pharmaceutical composition comprising the compound of formula I according to claim 1 or a pharmaceutically acceptable salt thereof, or an enantiomer, a diastereomer, a tautomer, a torsional isomer, a solvate, a polymorph or a prodrug thereof, characterized in that the pharmaceutical composition comprises:
(i) an effective amount of the compound of formula I, or a pharmaceutically acceptable salt thereof, or an enantiomer, a diastereomer, a tautomer, a solvate, a polymorph or a prodrug thereof; and
(ii) a pharmaceutically acceptable carrier.
